(19) United States
(12) Patent Application Publication
(10) Pub. No.: US 2026/0195893 A1
(43) Pub. Date: Jul. 9, 2026

(54) DYNAMIC TOOTH CHART AND AUTOMATIC CHARTING

(71) Applicant: Align Technology, Inc., San Jose, CA (US)

(72) Inventors: Michael Sabina, Campbell, CA (US); Michael Austin Brown, Orlando, FL (US); Christopher E. Cramer, Durham, NC (US); Ran Katz, Hod Hasharon (IL); Tomer Peled, Ness-Zionna (IL)

(21) Appl. No.: 19/430,058

(22) Filed: Dec. 22, 2025

Related U.S. Application Data
(60) Provisional application No. 63/742,611, filed on Jan. 7, 2025.

(51) Int. Cl. G06T 7/00; G16H 50/20

(52) U.S. Cl. CPC G06T 7/0012; G16H 50/20; G06T 2207/10; G06T 2207/20081; G06T 2207/30036

(57) ABSTRACT

A method is provided for generating and/or displaying a dynamic tooth chart. In some cases, the method can include receiving dataset(s) associated with a patient. Each dataset comprises data corresponding to an imaging modality of a plurality of imaging modalities and provides information on a dentition of the patient. The datasets are processed to generate, for each dataset, one or more tooth portions. Each tooth portion comprises an image of one or more teeth of the patient. The one or more tooth portions are arranged into a dental chart. A visualization of the dental chart of the patient is generated and provided to a user device for presentation in a user interface. In some cases, a method can include determining one or more chart annotations can by performing a shape analysis, a teeth arrangement analysis, and/or a material analysis on 3D dental model(s) and/or 2D images of the patient's jaw.

### RELATED APPLICATION

The present application claims priority to U.S. Provisional Patent Application No. 63/742,611, filed on Jan. 7, 2025, which is herein incorporated by reference in their entirety.

### TECHNICAL FIELD

The instant specification generally relates to systems and methods for generating and displaying a dynamic tooth chart, and for enabling automatic tooth charting.

### BACKGROUND

Dental charting is a fundamental tool used by dental professionals to record and assess the condition of a patient's teeth and supporting oral structures. A dental chart can include notations indicating, for example, the presence of caries, fillings, missing teeth, periodontal conditions, and other oral health conditions. Traditional dental charts rely on a combination of graphical representations and alphanumeric annotations to convey this information. These charts can serve as a clinical reference for ongoing patient care.

Over time, dental charting systems have evolved to accommodate advances in dental practice, including digital record-keeping and integration with diagnostic imaging technologies. Despite these advancements, many charting systems remain cumbersome to use, prone to inconsistencies in notation, and difficult to interpret across different practitioners or practice management systems. This has highlighted the need for a more standardized, intuitive, and interoperable charting format that enhances accuracy, usability, and communication within the dental profession.

### SUMMARY

The below summary is a simplified summary of the disclosure in order to provide a basic understanding of some aspects of the disclosure. This summary is not an extensive overview of the disclosure. It is intended neither to identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

In a first implementation, a method comprises receiving one or more datasets associated with a patient. Each data of the one or more datasets can correspond to an imaging modality of a plurality of imaging modalities and can provide information on a dentition of a patient. The method can further comprise processing the one or more datasets to generate, for each dataset of the one or more datasets, one or more tooth portions. Each tooth portion can comprise an image of one or more teeth of the patient. The method can further comprise arranging the one or more tooth portions into a dental chart. The method can further comprise generating a visualization of the dental chart of the patient. The method can further comprise providing, to a user device, the visualization of the dental chart for presentation in a user interface (UI).

A second implementation may further extend any of the first implementation. In the second implementation, the method may further include normalizing the one or more tooth portions.

A third implementation may further extend any of the second implementation. In the third implementation, the one or more tooth portions are normalized for at least one of a size, a scale, a color balance, a brightness, or an orientation for coordinated presentation on the dental chart.

A fourth implementation may further extend any of the first through third implementations. In the fourth implementation, the method may further include determining the imaging modality of the plurality of imaging modalities and selecting the one or more tooth portions corresponding to the determined imaging modality.

A fifth implementation may further extend any of the fourth implementation. In the fifth implementation, determining the imaging modality may further include identifying a correlation between a context of a dental visit and each of the plurality of imaging modalities; ranking each of the plurality of imaging modalities according to the correlation; and determining the imaging modality with a highest ranking.

A sixth implementation may further extend any of the fifth implementation. In the sixth implementation, the context of the dental visit corresponds to at least one of a complaint of the patient, an input of a user device, a treatment plan, a treatment type, a scheduled dental visit, or a detection of a condition.

A seventh implementation may further extend any of the first through sixth implementations. In the seventh implementation, the plurality of imaging modalities comprise at least one of an intraoral scan, a near-infrared, a cone bean computed tomography (CBCT), a photograph, a video, a radiograph, fluorescence, or optical coherence tomography.

An eighth implementation may further extend any of the first through seventh implementations. In the eighth implementation, the visualization of the dental chart comprises at least one of a buccal view (e.g., displaying the side of the teeth that faces the cheeks), an occlusal view (e.g., displaying the biting or chewing surface of the teeth), or a lingual view (e.g., displaying the side of the teeth that faces the longue) of a dental arch of the patient.

A ninth implementation may further extend any of the first through eighth implementations. In the ninth implementation, the visualization of the dental chart displays a dental arch of the patient in at least one of a two-dimensional view, a three-dimensional view, or a multi-dimensional view.

A tenth implementation may further extend any of the first through ninth implementations. In the tenth implementation, the visualization comprises two or more imaging modalities.

An eleventh implementation may further extend any of the first through tenth implementations. In the eleventh implementation, a first tooth portion of the one or more tooth portions has a first imaging modality, and a second tooth portion of the one or more tooth portions has a second imaging modality.

A twelfth implementation may further extend any of the first through eleventh implementations. In the twelfth implementation, the method may further include receiving, from the user device, a user interaction associated with the visualization of the dental chart; identifying, base don the user interaction, a second imaging modality of the plurality of imaging modalities; and updating the visualization of the dental chart of the patient by selecting the one or more tooth portions that each corresponds to the second imaging modality.

A thirteenth implementation may further extend any of the first through twelfth implementations. In the thirteenth implementation, the method may further include processing the at least of the one or more datasets using a trained artificial intelligence (AI) model that outputs one or more detected oral conditions; and updating the one or more tooth portions to include a representation of the one or more detected oral conditions detected for one or more teeth represented in the one or more tooth portions.

A fourteenth implementation may further extend any of the first through thirteenth implementations. In the fourteenth implementation, each dataset comprises data from a particular point in time.

A fifteenth implementation may further extend any of the first through fourteenth implementations. In the fifteenth implementation, the visualization corresponds to a particular point in time.

A sixteenth implementation may further extend any of the first through fifteenth implementations. In the sixteenth implementation, the UI enables a user of the user device to interact with the visualization by at least one of focusing in a particular section of a dental arch, changing imaging modalities, or changing a point in time.

A seventeenth implementation may further extend any of the first through sixteenth implementations. In the seventeenth implementation, the method may further include providing, as input, the one or more datasets to an artificial intelligence model trained to provide an indication of a clinical finding corresponding to a particular imaging modality of the plurality of imaging modalities; receiving, from the artificial intelligence model, the indication of the clinical finding corresponding to the particular imaging modality; and including the clinical finding in the visualization of the dental chart of the patient.

An eighteenth implementation may further extend any of the seventeenth implementation. In the eighteenth implementation, including the clinical finding in the visualization of the dental chart can further include determining a second imaging modality of the plurality of imaging modalities other than the particular imaging modality; and selecting the one or more tooth portions corresponding to the second imaging modality.

A nineteenth implementation may further extend any of the eighteenth implementation. In the nineteenth implementation, determining the second imaging modality can further include identifying a correlation between the clinical finding and each of the plurality of imaging modalities; ranking each of the plurality of imaging modalities; and determining the second imaging modality with a highest ranking.

A twentieth implementation may further extend any of the first through nineteenth implementations. In the twentieth implementation, the method may further include receiving, from at least one artificial intelligence model, a plurality of indications of a clinical finding, wherein each indication corresponds to a particular imaging modality of the plurality of imaging modalities.

A twenty-first implementation may further extend any of the twentieth implementation. In the twenty-first implementation, the method may further include identifying a primary imaging modality corresponding to the clinical finding; identifying a primary indication of the plurality of indications, wherein the primary indication corresponds to the primary imaging modality; and including the primary indication in the visualization of the dental chart of the patient.

A twenty-second implementation may further extend any of the twentieth through twenty-first implementations. In the twenty-second implementation, the method may further include generating an overall indication of the clinical finding by aggregating the plurality of indications of the clinical finding; and including the overall indication of the clinal finding in the visualization of the dental chart of the patient.

A twenty-third implementation may further extend any of the first through twenty-second implementations. In the twenty-third implementation, the data corresponding to the imaging modality comprises data associated with a plurality of image sources.

A twenty-fourth implementation may further extend any of the twenty-third implementation. In the twenty-fourth implementation, the method may further include providing, as input, the image data associated with the plurality of image sources to an artificial intelligence model trained to provide a confidence score associated with an identified clinical finding for each of the plurality of image sources; receiving, as output form the artificial intelligence model, a plurality of confidence scores associated with a clinical finding for the plurality of image sources; identifying an image source of the plurality of image sources with a highest confidence score; and including, in the visualization of the dental chart, the image source with the highest confidence score.

A twenty-fifth implementation may further extend any of the first through twenty-fourth implementations. In the twenty-fifth implementation, the visualization comprises a first portion displaying a dental arch of the patient in a panoramic format and a second portion displaying the dental arch of the patient in an arch format.

A twenty-sixth implementation may further extend any of the first through twenty-fourth implementations. In twenty-sixth implementation, the method may further comprise receiving, from the user device, via one or more controls of the UI, a modification to a measurement of a tooth of the one or more teeth of the patient; generating one or more updated tooth portions to reflect the modification; generating an updated visualization of the dental chart to include the one or more updated tooth portions; and providing, to the user device, the updated visualization of the dental chart.

In a twenty-seventh implementation, a system comprises a memory and a processing device to execute instructions from the memory. The processing device is configured to receive one or more datasets associated with a patient, wherein each dataset of the one or more datasets comprises data corresponding to an imaging modality of a plurality of imaging modalities and provides information on a dentition of the patient. The processing device is further configured to process the one or more datasets to generate, for each dataset of the one or more datasets, one or more tooth portions, wherein each tooth portion comprises an image of one or more teeth of the patient. The processing device is further configured to arrange the one or more tooth portions into a dental chart. The processing device is further configured to generate a visualization of the dental chart of the patient. The processing device is further configured to provide, to a user device, the visualization of the dental chart for presentation in a user interface (UI).

A twenty-eighth implementation may further extend any of the twenty-seventh implementation. In the twenty-eighth implementation, the processing device is further configured to normalize the one or more tooth portions.

A twenty-ninth implementation may further extend any of the twenty-eighth implementation. In the twenty-ninth implementation, the one or more tooth portions are normalized for at least one of a size, a scale, a color balance, a brightness, or an orientation for coordinated presentation on the dental chart.

A thirtieth implementation may further extend any of the twenty-seventh through twenty-ninth implementations. In the thirtieth implementation, the processing device is further configured to determine the imaging modality of the plurality of imaging modalities and select the one or more tooth portions corresponding to the determined imaging modality.

A thirty-first implementation may further extend any of the thirtieth implementation. In the thirty-first implementation, to determine the imaging modality, the processing device is further configured to identify a correlation between a context of a dental visit and each of the plurality of imaging modalities; rank each of the plurality of imaging modalities according to the correlation; and determine the imaging modality with a highest ranking.

A thirty-second implementation may further extend any of the thirty-first implementation. In the thirty-second implementation, the context of the dental visit corresponds to at least one of a complaint of the patient, an input of a user of the user device, a treatment plan, a treatment type, a scheduled dental visit, or a detection of a condition.

A thirty-third implementation may further extend any of the twenty-seventh through thirty-second implementations. In the thirty-third implementation, the plurality of imaging modalities comprise at least one of: intraoral scan, near-infrared, cone beam computed tomography (CBCT), photograph, video, radiograph, fluorescence, or optical coherence tomography (OCT).

A thirty-fourth implementation may further extend any of the twenty-seventh through thirty-third implementations. In the thirty-fourth implementation, the visualization of the dental chart comprises at least one of a buccal view, an occlusal view, or a lingual view of a dental arch of the patient.

A thirty-fifth implementation may further extend any of the twenty-seventh through thirty-fourth implementations. In the thirty-fifth implementation, the visualization of the dental chart displays a dental arch of the patient in at least one of a two-dimensional view, a three-dimensional view, or a multi-dimensional view.

A thirty-sixth implementation may further extend any of the twenty-seventh through thirty-fifth implementations. In the thirty-sixth implementation, the visualization comprises two or more imaging modalities.

A thirty-seventh implementation may further extend any of the twenty-seventh through thirty-sixth implementations. In the thirty-seventh implementation, a first tooth portion of the one or more tooth portions has a first imaging modality and a second tooth portion of the one or more tooth portions has a second imaging modality.

A thirty-eighth implementation may further extend any of the twenty-seventh through thirty-seventh implementations. In the thirty-eighth implementation, the processing device is further configured to receive, from the user device, a user interaction associated with the visualization of the dental chart; identify, based on the user interaction, a second imaging modality of the plurality of imaging modalities; and update the visualization of the dental chart of the patient by selecting the one or more tooth portions that each corresponds to the second imaging modality.

A thirty-ninth implementation may further extend any of the twenty-seventh through thirty-eighth implementations. In the thirty-ninth implementation, the processing device is further configured to process the at least one of the one or more datasets using a trained artificial intelligence (AI) model that outputs one or more detected oral conditions; and update the one or more tooth portions to include a representation of the one or more detected oral conditions detected for one or more teeth represented in the one or more tooth portions.

A fortieth implementation may further extend any of the twenty-seventh through thirty-ninth implementations. In the fortieth implementation, each dataset comprises data from a particular point in time.

A forty-first implementation may further extend any of the twenty-seventh through fortieth implementations. In the forty-first implementation, the visualization corresponds to a particular point in time.

A forty-second implementation may further extend any of the twenty-seventh through forty-first implementations. In the forty-second implementation, the UI enables a user of the user device to interact with the visualization by at least one of focusing in a particular section of a dental arch, changing imaging modalities, or changing a point in time.

A forty-third implementation may further extend any of the twenty-seventh through forty-second implementations. In the forty-third implementation, the processing device is further configured to provide, as input, the one or more datasets to an artificial intelligence model trained to provide an indication of a clinical finding corresponding to a particular imaging modality of the plurality of imaging modalities; receive, from the artificial intelligence model, the indication of the clinical finding corresponding to the particular imaging modality; and include the clinical finding in the visualization of the dental chart of the patient.

A forty-fourth implementation may further extend any of the forty-third implementation. In the forty-fourth implementation, to include the clinical finding in the visualization of the dental chart, the processing device is further configured to determine a second imaging modality of the plurality of imaging modalities other than the particular imaging modality; and select the one or more tooth portions corresponding to the second imaging modality.

A forty-fifth implementation may further extend any of the forty-fourth implementation. In the forty-fifth implementation, to determine the second imaging modality, the processing device is further configured to identify a correlation between the clinical finding and each of the plurality of imaging modalities; rank each of the plurality of imaging modalities according to the correlation; and determine the second imaging modality with a highest ranking.

A forty-sixth implementation may further extend any of the twenty-seventh through forty-fifth implementations. In the forty-sixth implementation, the processing device is further configured to receive, from at least one artificial intelligence model, a plurality of indications of a clinical finding, wherein each indication corresponds to a particular imaging modality of the plurality of imaging modalities.

A forty-seventh implementation may further extend any of the forty-sixth implementation. In the forty-seventh implementation, the processing device is further configured to identify a primary imaging modality corresponding to the clinical finding; identify a primary indication of the plurality of indications, wherein the primary indication corresponds to the primary imaging modality; and include the primary indication in the visualization of the dental chart of the patient.

A forty-eighth implementation may further extend any of the forty-sixth through forty-seventh implementations. In the forty-eighth implementation, the processing device is further configured to generate an overall indication of the clinical finding by aggregating the plurality of indications of the clinical finding; and include the overall indication of the clinical finding in the visualization of the dental chart of the patient.

A forty-ninth implementation may further extend any of the twenty-seventh through forty-eighth implementations. In the forty-ninth implementation, the data corresponding to the imaging modality comprises image data associated with a plurality of image sources.

A fiftieth implementation may further extend any of the forty-ninth implementation. In the fiftieth implementation, the processing device is further configured to provide, as input, the image data associated with the plurality of image sources to an artificial intelligence model trained to provide a confidence score associated with an identified clinical finding for each of the plurality of image sources; receive, as output from the artificial intelligence model, a plurality of confidence scores associated with a clinical finding for the plurality of image sources; identify an image source of the plurality of image sources with a highest confidence score; and include, in the visualization of the dental chart, the image source with the highest confidence score.

A fifty-first implementation may further extend any of the twenty-seventh through fiftieth implementations. In the fifty-first implementation, the visualization comprises a first portion displaying a dental arch of the patient in a panoramic format and a second portion displaying the dental arch of the patient in an arch format.

A fifty-second implementation may further extend any of the twenty-seventh through fifty-first implementations. In the fifty-second implementation, the processing device is further configured to receive, from the user device, via one or more controls of the UI, a modification to a measurement of a tooth of the one or more teeth of the patient; generate one or more updated tooth portions to reflect the modification; generate an updated visualization of the dental chart to include the one or more updated tooth portions; and provide, to the user device, the updated visualization of the dental chart.

In a fifty-third implementation, a non-transitory computer-readable storage medium comprises instructions that, when executed by a processing device, cause the processing device to receive one or more datasets associated with a patient, wherein each dataset of the one or more datasets comprises data corresponding to an imaging modality of a plurality of imaging modalities and provides information on a dentition of the patient. The instructions further cause the processing device to process the one or more datasets to generate, for each dataset of the one or more datasets, one or more tooth portions, wherein each tooth portion comprises an image of one or more teeth of the patient. The instructions further cause the processing device to arrange the one or more tooth portions into a dental chart. The instructions further cause the processing device to generate a visualization of the dental chart of the patient. The instructions further cause the processing device to provide, to a user device, the visualization of the dental chart for presentation in a user interface (UI).

A fifty-fourth implementation may further extend any of the fifty-third implementation. In the fifty-fourth implementation, the processing device is further caused to normalize the one or more tooth portions.

A fifty-fifth implementation may further extend any of the fifty-fourth implementation. In the fifty-fifth implementation, the one or more tooth portions are normalized for at least one of a size, a scale, a color balance, a brightness, or an orientation for coordinated presentation on the dental chart.

A fifty-sixth implementation may further extend any of the fifty-third through fifty-fifth implementations. In the fifty-sixth implementation, the processing device is further caused to determine the imaging modality of the plurality of imaging modalities; and select the one or more tooth portions corresponding to the determined imaging modality.

A fifty-seventh implementation may further extend any of the fifty-sixth implementation. In the fifty-seventh implementation, to determine the imaging modality, the processing device is further caused to identify a correlation between a context of a dental visit and each of the plurality of imaging modalities; rank each of the plurality of imaging modalities according to the correlation; and determine the imaging modality with a highest ranking.

A fifty-eighth implementation may further extend any of the fifty-seventh implementation. In the fifty-eighth implementation, the context of the dental visit corresponds to at least one of a complaint of the patient, an input of a user of the user device, a treatment plan, a treatment type, a scheduled dental visit, or a detection of a condition.

A fifty-ninth implementation may further extend any of the fifty-third through fifty-eighth implementations. In the fifty-ninth implementation, the plurality of imaging modalities comprise at least one of: intraoral scan, near-infrared, cone beam computed tomography (CBCT), photograph, video, radiograph, fluorescence, or optical coherence tomography (OCT).

A sixtieth implementation may further extend any of the fifty-third through fifty-ninth implementations. In the sixtieth implementation, the visualization of the dental chart comprises at least one of a buccal view, an occlusal view, or a lingual view of a dental arch of the patient.

A sixty-first implementation may further extend any of the fifty-third through sixtieth implementations. In the sixty-first implementation, the visualization of the dental chart displays a dental arch of the patient in at least one of a two-dimensional view, a three-dimensional view, or a multi-dimensional view.

A sixty-second implementation may further extend any of the fifty-third through sixty-first implementations. In the sixty-second implementation, the visualization comprises two or more imaging modalities.

A sixty-third implementation may further extend any of the fifty-third through sixty-second implementations. In the sixty-third implementation, a first tooth portion of the one or more tooth portions has a first imaging modality and a second tooth portion of the one or more tooth portions has a second imaging modality.

A sixty-fourth implementation may further extend any of the fifty-third through sixty-third implementations. In the sixty-fourth implementation, the processing device is further caused to receive, from the user device, a user interaction associated with the visualization of the dental chart; identify, based on the user interaction, a second imaging modality of the plurality of imaging modalities; and update the visualization of the dental chart of the patient by selecting the one or more tooth portions that each corresponds to the second imaging modality.

A sixty-fifth implementation may further extend any of the fifty-third through sixty-fourth implementations. In the sixty-fifth implementation, the processing device is further caused to process the at least one of the one or more datasets using a trained artificial intelligence (AI) model that outputs one or more detected oral conditions; and update the one or more tooth portions to include a representation of the one or more detected oral conditions detected for one or more teeth represented in the one or more tooth portions.

A sixty-sixth implementation may further extend any of the fifty-third through sixty-fifth implementations. In the sixty-sixth implementation, each dataset comprises data from a particular point in time.

A sixty-seventh implementation may further extend any of the fifty-third through sixty-sixth implementations. In the sixty-seventh implementation, the visualization corresponds to a particular point in time.

A sixty-eighth implementation may further extend any of the fifty-third through sixty-seventh implementations. In the sixty-eighth implementation, the UI enables a user of the user device to interact with the visualization by at least one of focusing in a particular section of a dental arch, changing imaging modalities, or changing a point in time.

A sixty-ninth implementation may further extend any of the fifty-third through sixty-eighth implementations. In the sixty-ninth implementation, the processing device is further caused to provide, as input, the one or more datasets to an artificial intelligence model trained to provide an indication of a clinical finding corresponding to a particular imaging modality of the plurality of imaging modalities; receive, from the artificial intelligence model, the indication of the clinical finding corresponding to the particular imaging modality; and include the clinical finding in the visualization of the dental chart of the patient.

A seventieth implementation may further extend any of the sixty-ninth implementation. In the seventieth implementation, to include the clinical finding in the visualization of the dental chart, the processing device is further caused to determine a second imaging modality of the plurality of imaging modalities other than the particular imaging modality; and select the one or more tooth portions corresponding to the second imaging modality.

A seventy-first implementation may further extend any of the seventieth implementation. In the seventy-first implementation, to determine the second imaging modality, the processing device is further caused to identify a correlation between the clinical finding and each of the plurality of imaging modalities; rank each of the plurality of imaging modalities according to the correlation; and determine the second imaging modality with a highest ranking.

A seventy-second implementation may further extend any of the fifty-third through seventy-first implementations. In the seventy-second implementation, the processing device is further caused to receive, from at least one artificial intelligence model, a plurality of indications of a clinical finding, wherein each indication corresponds to a particular imaging modality of the plurality of imaging modalities.

A seventy-third implementation may further extend any of the seventy-second implementation. In the seventy-third implementation, the processing device is further caused to identify a primary imaging modality corresponding to the clinical finding; identify a primary indication of the plurality of indications, wherein the primary indication corresponds to the primary imaging modality; and include the primary indication in the visualization of the dental chart of the patient.

A seventy-fourth implementation may further extend any of the seventy-second through seventy-third implementations. In the seventy-fourth implementation, the processing device is further caused to generate an overall indication of the clinical finding by aggregating the plurality of indications of the clinical finding; and include the overall indication of the clinical finding in the visualization of the dental chart of the patient.

A seventy-fifth implementation may further extend any of the fifty-third through seventy-fourth implementations. In the seventy-fifth implementation, the data corresponding to the imaging modality comprises image data associated with a plurality of image sources.

A seventy-sixth implementation may further extend any of the seventy-fifth implementation. In the seventy-sixth implementation, the processing device is further caused to provide, as input, the image data associated with the plurality of image sources to an artificial intelligence model trained to provide a confidence score associated with an identified clinical finding for each of the plurality of image sources; receive, as output from the artificial intelligence model, a plurality of confidence scores associated with a clinical finding for the plurality of image sources; identify an image source of the plurality of image sources with a highest confidence score; and include, in the visualization of the dental chart, the image source with the highest confidence score.

A seventy-seventh implementation may further extend any of the fifty-third through seventy-sixth implementations. In the seventy-seventh implementation, the visualization comprises a first portion displaying a dental arch of the patient in a panoramic format and a second portion displaying the dental arch of the patient in an arch format.

A seventy-eighth implementation may further extend any of the fifty-third through seventy-seventh implementations. In the seventy-eighth implementation, the processing device is further caused to receive, from the user device, via one or more controls of the UI, a modification to a measurement of a tooth of the one or more teeth of the patient; generate one or more updated tooth portions to reflect the modification; generate an updated visualization of the dental chart to include the one or more updated tooth portions; and provide, to the user device, the updated visualization of the dental chart.

In a seventy-ninth implementation, a method comprises identifying one or more datasets associated with a dental arch of a patient, wherein each of the one or more datasets corresponds to an imaging modality of a plurality of imaging modalities. The method further comprises segmenting each dataset into a plurality of sections, wherein each section of the plurality of sections corresponds to a portion of the dental arch of the patient. The method further comprises generating a corresponding normalized section of each dataset by normalizing, for each dataset, each section of the plurality of sections. The method further comprises generating, for each section of the plurality of sections, a tooth portion comprising the corresponding normalized section of each dataset.

An eightieth implementation may further extend any of the seventy-ninth implementation. In the eightieth implementation, the portion of the dental arch corresponds to one or more teeth of the dental arch of the patient.

An eighty-first implementation may further extend any of the seventy-ninth through eightieth implementations. In the eighty-first implementation, the plurality of imaging modalities comprise at least one of:
intraoral scan, near-infrared, cone beam computed tomography (CBCT), photograph, video, radiograph, fluorescence, or optical coherence tomography (OCT).

An eighty-second implementation may further extend any of the seventy-ninth through eighty-first implementations. In the eighty-second implementation, a dataset of the one or more datasets corresponds to one of a buccal view, an occlusal view, or a lingual view of the dental arch of the patient.

An eighty-third implementation may further extend any of the seventy-ninth through eighty-second implementations. In the eighty-third implementation, each section is normalized for at least one of a size, a scale, a color balance, a brightness, or an orientation.

In an eighty-fourth implementation, a system comprises a memory and a processing device to execute instructions from the memory. The processing device is configured to identify one or more datasets associated with a dental arch of a patient, wherein each of the one or more datasets corresponds to an imaging modality of a plurality of imaging modalities. The processing device is further configured to segment each dataset into a plurality of sections, wherein each section of the plurality of sections corresponds to a portion of the dental arch of the patient. The processing device is further configured to generate a corresponding normalized section of each dataset by normalizing, for each dataset, each section of the plurality of sections. The processing device is further configured to generate, for each section of the plurality of sections, a tooth portion comprising the corresponding normalized section of each dataset.

An eighty-fifth implementation may further extend any of the eighty-fourth implementation. In the eighty-fifth implementation, the portion of the dental arch corresponds to one or more teeth of the dental arch of the patient.

An eighty-sixth implementation may further extend any of the eighty-fourth through eighty-fifth implementations. In the eighty-sixth implementation, the plurality of imaging modalities comprise at least one of: intraoral scan, near-infrared, cone beam computed tomography (CBCT), photograph, video, radiograph, fluorescence, or optical coherence tomography (OCT).

An eighty-seventh implementation may further extend any of the eighty-fourth through eighty-sixth implementations. In the eighty-seventh implementation, a dataset of the one or more datasets corresponds to one of a buccal view, an occlusal view, or a lingual view of the dental arch of the patient.

An eighty-eighth implementation may further extend any of the eighty-fourth through eighty-seventh implementations. In the eighty-eighth implementation, each section is normalized for at least one of a size, a scale, a color balance, a brightness, or an orientation.

In an eighty-ninth implementation, a non-transitory computer-readable storage medium comprises instructions that, when executed by a processing device, cause the processing device to identify one or more datasets associated with a dental arch of a patient, wherein each of the one or more datasets corresponds to an imaging modality of a plurality of imaging modalities. The instructions further cause the processing device to segment each dataset into a plurality of sections, wherein each section of the plurality of sections corresponds to a portion of the dental arch of the patient. The instructions further cause the processing device to generate a corresponding normalized section of each dataset by normalizing, for each dataset, each section of the plurality of sections. The instructions further cause the processing device to generate, for each section of the plurality of sections, a tooth portion comprising the corresponding normalized section of each dataset.

A ninetieth implementation may further extend any of the eighty-ninth implementation. In the ninetieth implementation, the portion of the dental arch corresponds to one or more teeth of the dental arch of the patient.

A ninety-first implementation may further extend any of the eighty-ninth through ninetieth implementations. In the ninety-first implementation, the plurality of imaging modalities comprise at least one of: intraoral scan, near-infrared, cone beam computed tomography (CBCT), photograph, video, radiograph, fluorescence, or optical coherence tomography (OCT).

A ninety-second implementation may further extend any of the eighty-ninth through ninety-first implementations. In the ninety-second implementation, a dataset of the one or more datasets corresponds to one of a buccal view, an occlusal view, or a lingual view of the dental arch of the patient.

A ninety-third implementation may further extend any of the eighty-ninth through ninety-second implementations. In the ninety-third implementation, each section is normalized for at least one of a size, a scale, a color balance, a brightness, or an orientation.

In a ninety-fourth implementation, a method comprises receiving scan data comprising at least one of a three-dimensional (3D) model of a jaw of a patient, one or more two-dimensional (2D) color intraoral images of the patient, or one or more near-infrared imaging (NIRI) intraoral images of the patient. The method further comprises identifying a subset of the scan data for each tooth of the patient. The method further comprises determining one or more chart annotations for a dental chart of the patient, wherein the one or more chart annotations are determined by at least one of: performing, for each subset of the scan data, a shape analysis to identify a status of the tooth of the patient; performing, for each subset of the scan data, a teeth arrangement analysis to identify a positioning of the tooth of the patient; or performing, for each subset of the scan data, a material classification to identify a composition of the tooth of the patient.

A ninety-fifth implementation may further extend any of the ninety-fourth implementation. In the ninety-fifth implementation, identifying the subset of the scan data for each tooth of the patient comprises at least one of:
segmenting the 3D model to generate a series of secondary 3D models, wherein each secondary 3D model in the series of secondary 3D models corresponds to a tooth of the patient; identifying a portion of the one or more 2D color intraoral images corresponding to the tooth of the patient; or identifying a portion of the one or more NIRI intraoral images corresponding to the tooth of the patient.

A ninety-sixth implementation may further extend any of the ninety-fifth implementation. In the ninety-sixth implementation, performing the teeth arrangement analysis comprises: generating, based on the 3D model, a jaw line reference curve for the patient; identifying, based on segmentation data of the secondary 3D model, a location of the tooth; making a comparison of the location of the tooth to the jaw line reference curve; and determining, based on the comparison, the positioning of the tooth, wherein the positioning comprises one of a regular positioning, an irregular positioning, or an indication of a missing tooth.

A ninety-seventh implementation may further extend any of the ninety-fifth implementation. In the ninety-seventh implementation, the method may further comprise comparing a first secondary 3D model corresponding to a first tooth of the patient to a second secondary 3D model corresponding to a second tooth of the patient, wherein the second tooth is symmetrically opposite the first tooth in the jaw of the patient; determining, based on the comparison, an asymmetry metric representing a dissimilarity between the first tooth and the second tooth; and adding the asymmetry metric to at least one of the subset of the scan data for the first tooth or the subset of the scan data for the second tooth.

A ninety-eighth implementation may further extend any of the ninety-seventh implementation. In the ninety-eighth implementation, the method may further comprise responsive to determining that the asymmetry metric satisfies a condition, performing an error recovery operation corresponding to the dissimilarity between the first tooth and the second tooth.

A ninety-ninth implementation may further extend any of the ninety-fourth through ninety-eighth implementations. In the ninety-ninth implementation, the method may further comprise providing, to a user device, the one or more chart annotations to include in the dental chart of the patient; and providing the one or more chart annotations to a dental practice management system.

A one hundredth implementation may further extend any of the ninety-fourth through ninety-ninth implementations. In the one hundredth implementation, performing the shape analysis comprises: providing the subset of the scan data as input to a machine learning model, wherein the machine learning model outputs one or more indicators of the status of the tooth, the one or more indicators comprising at least one of a tooth number indicator, a primary tooth indicator, an abutment indicator, a scan-body indicator, a preparation tooth indicator, a partially hatched tooth indicator, a bracket indicator, an attachment indicator, a retainer indicator, or a braces indicator.

A one hundred first implementation may further extend any of the ninety-fourth through one hundredth implementations. In the one hundred first implementation, performing the shape analysis comprises: providing the subset of the scan data as input to a machine learning model, wherein the machine learning model is trained to provide, as output, at least one of a tooth identification, an outlier shape, or one or more indicators of dental accessories; and receiving, as output from the machine learning model, at least one of the tooth identifications, the outlier shape, or the one or more indicators of dental accessories.

A one hundred second implementation may further extend any of the one hundred first implementation. In the one hundred second implementation, the method may further comprise providing at least one of the tooth identification or the outlier shape to a classifier, wherein the classifier outputs one or more indicators of the status of the tooth, the one or more indicators comprising at least one of a tooth number indicator, a primary tooth indicator, an abutment indicator, a scan-body indicator, a preparation tooth indicator, or a partially hatched tooth indicator.

A one hundred third implementation may further extend any of the one hundred first through one hundred second implementations. In the one hundred third implementation, the method may further comprise providing the one or more indicators of dental accessories as input to a second machine learning model, wherein the second machine learning model outputs one or more additional indicators comprising at least one of a bracket indicator, an attachment indicator, a retainer indicator, or a braces indicator.

A one hundred fourth implementation may further extend any of the ninety-fourth through one hundred third implementations. In the one hundred fourth implementation, performing the material classification comprises: providing the subset of the scan data as input to a machine learning model, wherein the machine learning model outputs a classification of at least one of a partial restoration, a veneer, an inlay, an onlay, an overlay, a filling, a full restoration crown, or a bridge.

A one hundred fifth implementation may further extend any of the one hundred fourth implementation. In the one hundred fifth implementation, the method may further comprise segmenting the classification of the at least one of the partial restoration, the veneer, the inlay, the onlay, the overlay, the filling, the full restoration crown, or the bridge from the corresponding tooth, wherein the segmentation provides at least one of a location or a size corresponding to the classification of the at least one of the partial restoration, the veneer, the inlay, the onlay, the overlay, the filling, the full restoration crown, or the bridge.

A one hundred sixth implementation may further extend any of the ninety-fourth through one hundred fifth implementations. In the one hundred sixth implementation, the method may further comprise providing the one or more chart annotations to a diagnostic system, wherein an outcome of the diagnostic system comprises the one or more chart annotations.

A one hundred seventh implementation may further extend any of the ninety-fourth through one hundred sixth implementations. In the one hundred seventh implementation, the method may further comprise identifying, for the tooth of the patient, a caries indication identified by a caries diagnostic system; and responsive to determining that the one or more chart annotations for the tooth of the patient satisfies a caries condition, providing an updated caries identification to the caries diagnostic system.

A one hundred eighth implementation may further extend any of the ninety-fourth through one hundred seventh implementations. In the one hundred eighth implementation, the method may further comprise responsive to identifying, based on the one or more chart annotations, that the tooth is a restoration, providing an indication of the restoration to a timelapse diagnostic system.

A one hundred ninth implementation may further extend any of the ninety-fourth through one hundred eighth implementations. In the one hundred ninth implementation, the method may further comprise responsive to identifying, based on the one or more chart annotations, that the patient is in active orthodontic treatment, providing an indication of the active orthodontic treatment to a timelapse diagnostic system.

A one hundred tenth implementation may further extend any of the ninety-fourth through one hundred ninth implementations. In the one hundred tenth implementation, the method may further comprise responsive to identifying, based on the one or more chart annotations, that the tooth is an implant, providing an indication of the implant to a tooth strength assessment system.

A one hundred eleventh implementation may further extend any of the ninety-fourth through one hundred tenth implementations. In the one hundred eleventh implementation, the method may further comprise determining an orthodontic treatment plan for the patient, wherein the orthodontic treatment plan comprises a first location of a dental attachment for the tooth of the patient; identifying, based on the one or more chart annotations, a second location of the dental attachment for the tooth of the patient; and responsive to determining that the first location and the second location differ, providing, to a user device, an indication of a displacement of the dental attachment.

A one hundred twelfth implementation may further extend any of the ninety-fourth through one hundred eleventh implementations. In the one hundred twelfth implementation, the method may further comprise validating a first set of the one or more chart annotations corresponding to a first scan of the patient taken at a first point in time by comparing the first set of the one or more chart annotations to a second set of chart annotations corresponding to a second scan of the patient taken at a second point in time, wherein the second point in time predates the first point in time.

A one hundred thirteenth implementation may further extend any of the ninety-fourth through one hundred twelfth implementations. In the one hundred thirteenth implementation, the method may further comprise receiving second scan data comprising one or more geometrical connections between the 3D model and the at least one of the one or more 2D color intraoral images or the one or more NIRI intraoral images; responsive to segmenting the 3D model, projecting segmentation results onto the at least one of the one or more 2D color intraoral images or the one or more NIRI intraoral images; responsive to performing the shape analysis, mapping the status of the tooth to the at least one of the one or more 2D color intraoral images or the one or more NIRI intraoral images; responsive to performing the teeth arrangement analysis, mapping the positioning of the tooth to the at least one of the one or more 2D color intraoral images or the one or more NIRI intraoral images; and responsive to performing the material classification, mapping the composition of the tooth the 3D model.

A one hundred fourteenth implementation may further extend any of the ninety-fourth through one hundred thirteenth implementations. In the one hundred fourteenth implementation, determining the one or more chart annotations for the dental chart of the patient comprises: providing at least one of a secondary 3D model corresponding to the tooth of the patient, the one or more two-dimensional (2D) color intraoral images, or the one or more near-infrared imaging (NIRI) intraoral images as input to a machine learning model, wherein the machine learning model outputs one or more chart annotation indicators, wherein the one or more chart annotation indicators correspond to at least one of a status indicator, a positioning indicator, or a composition indicator.

A one hundred fifteenth implementation may further extend any of the one hundred fourteenth implementation. In the one hundred fifteenth implementation, the status indicator represents at least one of a tooth number indicator, a primary tooth indicator, an abutment indicator, a scan-body indicator, a preparation tooth indicator, a partially hatched tooth indicator, a bracket indicator, an attachment indicator, a retainer indicator, or a braces indicator.

A one hundred sixteenth implementation may further extend any of the one hundred fourteenth through one hundred fifteenth implementations. In the one hundred sixteenth implementation, the positioning indicator reflects one of a regular positioning, an irregular positioning, or an indication of a missing tooth.

A one hundred seventeenth implementation may further extend any of the one hundred fourteenth through one hundred sixteenth implementations. In the one hundred seventeenth implementation, the composition indicator reflects at least one of a partial restoration, a veneer, an inlay, an onlay, an overlay, a filling, a full restoration crown, or a bridge.

In a one hundred eighteenth implementation, a system comprises a memory and a processing device to execute instructions from the memory. The processing device is configured to receive scan data comprising at least one of a three-dimensional (3D) model of a jaw of a patient, one or more two-dimensional (2D) color intraoral images of the patient, or one or more near-infrared imaging (NIRI) intraoral images of the patient. The processing device is further configured to identify a subset of the scan data for each tooth of the patient. The processing device is further configured to determine one or more chart annotations for a dental chart of the patient, wherein the one or more chart annotations are determined by at least one of: performing, for each subset of the scan data, a shape analysis to identify a status of the tooth of the patient; performing, for each subset of the scan data, a teeth arrangement analysis to identify a positioning of the tooth of the patient; or performing, for each subset of the scan data, a material classification to identify a composition of the tooth of the patient.

A one hundred nineteenth implementation may further extend any of the one hundred eighteenth implementation. In the one hundred nineteenth implementation, to identify the subset of the scan data for each tooth of the patient, the instructions comprise at least one of: segmenting the 3D model to generate a series of secondary 3D models, wherein each secondary 3D model in the series of secondary 3D models corresponds to a tooth of the patient; identifying a portion of the one or more 2D color intraoral images corresponding to the tooth of the patient; or identifying a portion of the one or more NIRI intraoral images corresponding to the tooth of the patient.

A one hundred twentieth implementation may further extend any of the one hundred nineteenth implementation. In the one hundred twentieth implementation, performing the teeth arrangement analysis comprises:
generating, based on the 3D model, a jaw line reference curve for the patient; identifying, based on segmentation data of the secondary 3D model, a location of the tooth; making a comparison of the location of the tooth to the jaw line reference curve; and determining, based on the comparison, the positioning of the tooth, wherein the positioning comprises one of a regular positioning, an irregular positioning, or an indication of a missing tooth.

A one hundred twenty-first implementation may further extend any of the one hundred nineteenth through one hundred twentieth implementations. In the one hundred twenty-first implementation, the processing device is further configured to compare a first secondary 3D model corresponding to a first tooth of the patient to a second secondary 3D model corresponding to a second tooth of the patient, wherein the second tooth is symmetrically opposite the first tooth in the jaw of the patient; determine, based on the comparison, an asymmetry metric representing a dissimilarity between the first tooth and the second tooth; and add the asymmetry metric to at least one of the subset of the scan data for the first tooth or the subset of the scan data for the second tooth.

A one hundred twenty-second implementation may further extend any of the one hundred twenty-first implementation. In the one hundred twenty-second implementation, the processing device is further configured to, responsive to determining that the asymmetry metric satisfies a condition, perform an error recovery operation corresponding to the dissimilarity between the first tooth and the second tooth.

A one hundred twenty-third implementation may further extend any of the one hundred eighteenth through one hundred twenty-second implementations. In the one hundred twenty-third implementation, the processing device is further configured to provide, to a user device, the one or more chart annotations to include in the dental chart of the patient; and provide the one or more chart annotations to a dental practice management system.

A one hundred twenty-fourth implementation may further extend any of the one hundred eighteenth through one hundred twenty-third implementations. In the one hundred twenty-fourth implementation, performing the shape analysis comprises: providing the subset of the scan data as input to a machine learning model, wherein the machine learning model outputs one or more indicators of the status of the tooth, the one or more indicators comprising at least one of a tooth number indicator, a primary tooth indicator, an abutment indicator, a scan-body indicator, a preparation tooth indicator, a partially hatched tooth indicator, a bracket indicator, an attachment indicator, a retainer indicator, or a braces indicator.

A one hundred twenty-fifth implementation may further extend any of the one hundred eighteenth through one hundred twenty-fourth implementations. In the one hundred twenty-fifth implementation, performing the shape analysis comprises: providing the subset of the scan data as input to a machine learning model, wherein the machine learning model is trained to provide, as output, at least one of a tooth identification, an outlier shape, or one or more indicators of dental accessories; and receiving, as output from the machine learning model, at least one of the tooth identifications, the outlier shape, or the one or more indicators of dental accessories.

A one hundred twenty-sixth implementation may further extend any of the one hundred twenty-fifth implementation. In the one hundred twenty-sixth implementation, performing the shape analysis further comprises:
providing at least one of the tooth identification or the outlier shape to a classifier, wherein the classifier outputs one or more indicators of the status of the tooth, the one or more indicators comprising at least one of a tooth number indicator, a primary tooth indicator, an abutment indicator, a scan-body indicator, a preparation tooth indicator, or a partially hatched tooth indicator.

A one hundred twenty-seventh implementation may further extend any of the one hundred twenty-fifth through one hundred twenty-sixth implementations. In the one hundred twenty-seventh implementation, the processing device is further configured to provide the one or more indicators of dental accessories as input to a second machine learning model, wherein the second machine learning model outputs one or more additional indicators comprising at least one of a bracket indicator, an attachment indicator, a retainer indicator, or a braces indicator.

A one hundred twenty-eighth implementation may further extend any of the one hundred eighteenth through one hundred twenty-seventh implementations. In the one hundred twenty-eighth implementation, performing the material classification comprises: providing the subset of the scan data as input to a machine learning model, wherein the machine learning model outputs a classification of at least one of a partial restoration, a veneer, an inlay, an onlay, an overlay, a filling, a full restoration crown, or a bridge.

A one hundred twenty-ninth implementation may further extend any of the one hundred twenty-eighth implementation. In the one hundred twenty-ninth implementation, performing the material classification further comprises: segmenting the classification of the at least one of the partial restoration, the veneer, the inlay, the onlay, the overlay, the filling, the full restoration crown, or the bridge from the corresponding tooth, wherein the segmentation provides at least one of a location or a size corresponding to the classification of the at least one of the partial restoration, the veneer, the inlay, the onlay, the overlay, the filling, the full restoration crown, or the bridge.

A one hundred thirtieth implementation may further extend any of the one hundred eighteenth through one hundred twenty-ninth implementations. In the one hundred thirtieth implementation, the processing device is further configured to provide the one or more chart annotations to a diagnostic system, wherein an outcome of the diagnostic system comprises the one or more chart annotations.

A one hundred thirty-first implementation may further extend any of the one hundred eighteenth through one hundred thirtieth implementations. In the one hundred thirty-first implementation, the processing device is further configured to identify, for the tooth of the patient, a caries indication identified by a caries diagnostic system; and responsive to determining that the one or more chart annotations for the tooth of the patient satisfies a caries condition, provide an updated caries identification to the caries diagnostic system.

A one hundred thirty-second implementation may further extend any of the one hundred eighteenth through one hundred thirty-first implementations. In the one hundred thirty-second implementation, the processing device is further configured to, responsive to identifying, based on the one or more chart annotations, that the tooth is a restoration, provide an indication of the restoration to a timelapse diagnostic system.

A one hundred thirty-third implementation may further extend any of the one hundred eighteenth through one hundred thirty-second implementations. In the one hundred thirty-third implementation, the processing device is further configured to, responsive to identifying, based on the one or more chart annotations, that the patient is in active orthodontic treatment, provide an indication of the active orthodontic treatment to a timelapse diagnostic system.

A one hundred thirty-fourth implementation may further extend any of the one hundred eighteenth through one hundred thirty-third implementations. In the one hundred thirty-fourth implementation, the processing device is further configured to, responsive to identifying, based on the one or more chart annotations, that the tooth is an implant, provide an indication of the implant to a tooth strength assessment system.

A one hundred thirty-fifth implementation may further extend any of the one hundred eighteenth through one hundred thirty-fourth implementations. In the one hundred thirty-fifth implementation, the processing device is further configured to determine an orthodontic treatment plan for the patient, wherein the orthodontic treatment plan comprises a first location of a dental attachment for the tooth of the patient; identify, based on the one or more chart annotations, a second location of the dental attachment for the tooth of the patient; and responsive to determining that the first location and the second location differ, provide, to a user device, an indication of a displacement of the dental attachment.

A one hundred thirty-sixth implementation may further extend any of the one hundred eighteenth through one hundred thirty-fifth implementations. In the one hundred thirty-sixth implementation, the processing device is further configured to validate a first set of the one or more chart annotations corresponding to a first scan of the patient taken at a first point in time by comparing the first set of the one or more chart annotations to a second set of chart annotations corresponding to a second scan of the patient taken at a second point in time, wherein the second point in time predates the first point in time.

A one hundred thirty-seventh implementation may further extend any of the one hundred eighteenth through one hundred thirty-sixth implementations. In the one hundred thirty-seventh implementation, the processing device is further configured to receive second scan data comprising one or more geometrical connections between the 3D model and the at least one of the one or more 2D color intraoral images or the one or more NIRI intraoral images; responsive to segmenting the 3D model, project segmentation results onto the at least one of the one or more 2D color intraoral images or the one or more NIRI intraoral images; responsive to performing the shape analysis, map the status of the tooth to the at least one of the one or more 2D color intraoral images or the one or more NIRI intraoral images; responsive to performing the teeth arrangement analysis, map the positioning of the tooth to the at least one of the one or more 2D color intraoral images or the one or more NIRI intraoral images; and responsive to performing the material classification, map the composition of the tooth the 3D model.

A one hundred thirty-eighth implementation may further extend any of the one hundred eighteenth through one hundred thirty-seventh implementations. In the one hundred thirty-eighth implementation, to determine the one or more chart annotations for the dental chart of the patient, the processing device is further configured to provide at least one of a secondary 3D model corresponding to the tooth of the patient, the one or more two-dimensional (2D) color intraoral images, or the one or more near-infrared imaging (NIRI) intraoral images as input to a machine learning model, wherein the machine learning model outputs one or more chart annotation indicators, wherein the one or more chart annotation indicators correspond to at least one of a status indicator, a positioning indicator, or a composition indicator.

A one hundred thirty-ninth implementation may further extend any of the one hundred thirty-eighth implementation. In the one hundred thirty-ninth implementation, the status indicator represents at least one of a tooth number indicator, a primary tooth indicator, an abutment indicator, a scan-body indicator, a preparation tooth indicator, a partially hatched tooth indicator, a bracket indicator, an attachment indicator, a retainer indicator, or a braces indicator.

A one hundred fortieth implementation may further extend any of the one hundred thirty-eighth through one hundred thirty-ninth implementations. In the one hundred fortieth implementation, the positioning indicator reflects one of a regular positioning, an irregular positioning, or an indication of a missing tooth.

A one hundred forty-first implementation may further extend any of the one hundred thirty-eighth through one hundred fortieth implementations. In the one hundred forty-first implementation, the composition indicator reflects at least one of a partial restoration, a veneer, an inlay, an onlay, an overlay, a filling, a full restoration crown, or a bridge.

In a one hundred forty-second implementation, a non-transitory computer-readable storage medium comprises instructions that, when executed by a processing device, cause the processing device to receive scan data comprising at least one of a three-dimensional (3D) model of a jaw of a patient, one or more two-dimensional (2D) color intraoral images of the patient, or one or more near-infrared imaging (NIRI) intraoral images of the patient. The instructions further cause the processing device to identify a subset of the scan data for each tooth of the patient. The instructions further cause the processing device to determine one or more chart annotations for a dental chart of the patient, wherein the one or more chart annotations are determined by at least one of: performing, for each subset of the scan data, a shape analysis to identify a status of the tooth of the patient; performing, for each subset of the scan data, a teeth arrangement analysis to identify a positioning of the tooth of the patient; or performing, for each subset of the scan data, a material classification to identify a composition of the tooth of the patient.

A one hundred forty-third implementation may further extend any of the one hundred forty-second implementation. In the one hundred forty-third implementation, to identify the subset of the scan data for each tooth of the patient, the processing device is further caused to at least one of: segment the 3D model to generate a series of secondary 3D models, wherein each secondary 3D model in the series of secondary 3D models corresponds to a tooth of the patient; identify a portion of the one or more 2D color intraoral images corresponding to the tooth of the patient; or identify a portion of the one or more NIRI intraoral images corresponding to the tooth of the patient.

A one hundred forty-fourth implementation may further extend any of the one hundred forty-third implementation. In the one hundred forty-fourth implementation, performing the teeth arrangement analysis comprises: generating, based on the 3D model, a jaw line reference curve for the patient; identifying, based on segmentation data of the secondary 3D model, a location of the tooth; making a comparison of the location of the tooth to the jaw line reference curve; and determining, based on the comparison, the positioning of the tooth, wherein the positioning comprises one of a regular positioning, an irregular positioning, or an indication of a missing tooth.

A one hundred forty-fifth implementation may further extend any of the one hundred forty-third through one hundred forty-fourth implementations. In the one hundred forty-fifth implementation, the processing device is further caused to compare a first secondary 3D model corresponding to a first tooth of the patient to a second secondary 3D model corresponding to a second tooth of the patient, wherein the second tooth is symmetrically opposite the first tooth in the jaw of the patient; determine, based on the comparison, an asymmetry metric representing a dissimilarity between the first tooth and the second tooth; and add the asymmetry metric to at least one of the subset of the scan data for the first tooth or the subset of the scan data for the second tooth.

A one hundred forty-sixth implementation may further extend any of the one hundred forty-fifth implementation. In the one hundred forty-sixth implementation, the processing device is further caused to, responsive to determining that the asymmetry metric satisfies a condition, perform an error recovery operation corresponding to the dissimilarity between the first tooth and the second tooth.

A one hundred forty-seventh implementation may further extend any of the one hundred forty-second through one hundred forty-sixth implementations. In the one hundred forty-seventh implementation, the processing device is further caused to provide, to a user device, the one or more chart annotations to include in the dental chart of the patient; and provide the one or more chart annotations to a dental practice management system.

A one hundred forty-eighth implementation may further extend any of the one hundred forty-second through one hundred forty-seventh implementations. In the one hundred forty-eighth implementation, performing the shape analysis comprises: providing the subset of the scan data as input to a machine learning model, wherein the machine learning model outputs one or more indicators of the status of the tooth, the one or more indicators comprising at least one of a tooth number indicator, a primary tooth indicator, an abutment indicator, a scan-body indicator, a preparation tooth indicator, a partially hatched tooth indicator, a bracket indicator, an attachment indicator, a retainer indicator, or a braces indicator.

A one hundred forty-ninth implementation may further extend any of the one hundred forty-second through one hundred forty-eighth implementations. In the one hundred forty-ninth implementation, performing the shape analysis comprises: providing the subset of the scan data as input to a machine learning model, wherein the machine learning model is trained to provide, as output, at least one of a tooth identification, an outlier shape, or one or more indicators of dental accessories; and receiving, as out

A one hundred fiftieth implementation may further extend any of the one hundred forty-ninth implementation. In the one hundred fiftieth implementation, performing the shape analysis further comprises: providing at least one of the tooth identification or the outlier shape to a classifier, wherein the classifier outputs one or more indicators of the status of the tooth, the one or more indicators comprising at least one of a tooth number indicator, a primary tooth indicator, an abutment indicator, a scan-body indicator, a preparation tooth indicator, or a partially hatched tooth indicator.

A one hundred fifty-first implementation may further extend any of the one hundred forty-ninth through one hundred fiftieth implementations. In the one hundred fifty-first implementation, the processing device is further caused to provide the one or more indicators of dental accessories as input to a second machine learning model, wherein the second machine learning model outputs one or more additional indicators comprising at least one of a bracket indicator, an attachment indicator, a retainer indicator, or a braces indicator.

A one hundred fifty-second implementation may further extend any of the one hundred forty-second through one hundred fifty-first implementations. In the one hundred fifty-second implementation, performing the material classification comprises: providing the subset of the scan data as input to a machine learning model, wherein the machine learning model outputs a classification of at least one of a partial restoration, a veneer, an inlay, an onlay, an overlay, a filling, a full restoration crown, or a bridge.

A one hundred fifty-third implementation may further extend any of the one hundred fifty-second implementation. In the one hundred fifty-third implementation, performing the material classification further comprises: segmenting the classification of the at least one of the partial restoration, the veneer, the inlay, the onlay, the overlay, the filling, the full restoration crown, or the bridge from the corresponding tooth, wherein the segmentation provides at least one of a location or a size corresponding to the classification of the at least one of the partial restoration, the veneer, the inlay, the onlay, the overlay, the filling, the full restoration crown, or the bridge.

A one hundred fifty-fourth implementation may further extend any of the one hundred forty-second through one hundred fifty-third implementations. In the one hundred fifty-fourth implementation, the processing device is further caused to provide the one or more chart annotations to a diagnostic system, wherein an outcome of the diagnostic system comprises the one or more chart annotations.

A one hundred fifty-fifth implementation may further extend any of the one hundred forty-second through one hundred fifty-fourth implementations. In the one hundred fifty-fifth implementation, the processing device is further caused to identify, for the tooth of the patient, a caries indication identified by a caries diagnostic system; and responsive to determining that the one or more chart annotations for the tooth of the patient satisfies a caries condition, provide an updated caries identification to the caries diagnostic system.

A one hundred fifty-sixth implementation may further extend any of the one hundred forty-second through one hundred fifty-fifth implementations. In the one hundred fifty-sixth implementation, the processing device is further caused to, responsive to identifying, based on the one or more chart annotations, that the tooth is a restoration, provide an indication of the restoration to a timelapse diagnostic system.

A one hundred fifty-seventh implementation may further extend any of the one hundred forty-second through one hundred fifty-sixth implementations. In the one hundred fifty-seventh implementation, the processing device is further caused to, responsive to identifying, based on the one or more chart annotations, that the patient is in active orthodontic treatment, provide an indication of the active orthodontic treatment to a timelapse diagnostic system.

A one hundred fifty-eighth implementation may further extend any of the one hundred forty-second through one hundred fifty-seventh implementations. In the one hundred fifty-eighth implementation, the processing device is further caused to, responsive to identifying, based on the one or more chart annotations, that the tooth is an implant, provide an indication of the implant to a tooth strength assessment system.

A one hundred fifty-ninth implementation may further extend any of the one hundred forty-second through one hundred fifty-eighth implementations. In the one hundred fifty-ninth implementation, the processing device is further caused to determine an orthodontic treatment plan for the patient, wherein the orthodontic treatment plan comprises a first location of a dental attachment for the tooth of the patient; identify, based on the one or more chart annotations, a second location of the dental attachment for the tooth of the patient; and responsive to determining that the first location and the second location differ, provide, to a user device, an indication of a displacement of the dental attachment.

A one hundred sixtieth implementation may further extend any of the one hundred forty-second through one hundred fifty-ninth implementations. In the one hundred sixtieth implementation, the processing device is further caused to validate a first set of the one or more chart annotations corresponding to a first scan of the patient taken at a first point in time by comparing the first set of the one or more chart annotations to a second set of chart annotations corresponding to a second scan of the patient taken at a second point in time, wherein the second point in time predates the first point in time.

A one hundred sixty-first implementation may further extend any of the one hundred forty-second through one hundred sixtieth implementations. In the one hundred sixty-first implementation, the processing device is further caused to receive second scan data comprising one or more geometrical connections between the 3D model and the at least one of the one or more 2D color intraoral images or the one or more NIRI intraoral images; responsive to segmenting the 3D model, project segmentation results onto the at least one of the one or more 2D color intraoral images or the one or more NIRI intraoral images; responsive to performing the shape analysis, map the status of the tooth to the at least one of the one or more 2D color intraoral images or the one or more NIRI intraoral images; responsive to performing the teeth arrangement analysis, map the positioning of the tooth to the at least one of the one or more 2D color intraoral images or the one or more NIRI intraoral images; and responsive to performing the material classification, map the composition of the tooth the 3D model.

A one hundred sixty-second implementation may further extend any of the one hundred sixty-first implementation. In the one hundred sixty-second implementation, to determine the one or more chart annotations for the dental chart of the patient, the processing device is further caused to provide at least one of a secondary 3D model corresponding to the tooth of the patient, the one or more two-dimensional (2D) color intraoral images, or the one or more near-infrared imaging (NIRI) intraoral images as input to a machine learning model, wherein the machine learning model outputs one or more chart annotation indicators, wherein the one or more chart annotation indicators correspond to at least one of a status indicator, a positioning indicator, or a composition indicator.

A one hundred sixty-third implementation may further extend any of the one hundred sixty-second through one hundred sixty-second implementations. In the one hundred sixty-third implementation, the status indicator represents at least one of a tooth number indicator, a primary tooth indicator, an abutment indicator, a scan-body indicator, a preparation tooth indicator, a partially hatched tooth indicator, a bracket indicator, an attachment indicator, a retainer indicator, or a braces indicator.

A one hundred sixty-fourth implementation may further extend any of the one hundred sixty-second through one hundred sixty-third implementations. In the one hundred sixty-fourth implementation, the positioning indicator reflects one of a regular positioning, an irregular positioning, or an indication of a missing tooth.

A one hundred sixty-fifth implementation may further extend any of the one hundred sixty-second through one hundred sixty-fourth implementations. In the one hundred sixty-fifth implementation, the composition indicator reflects at least one of a partial restoration, a veneer, an inlay, an onlay, an overlay, a filling, a full restoration crown, or a bridge.

### BRIEF DESCRIPTION OF THE DRAWINGS

Aspects and embodiments of the present disclosure will be understood more fully from the detailed description given below and from the accompanying drawings of various aspects and embodiments of the disclosure, which, however, should not be taken to limit the disclosure to the specific aspects or embodiments, but are for explanation and understanding only.

FIG. 1 illustrates a workflow for detecting, predicting, diagnosing, and reporting on oral conditions and/or oral health problems, in accordance with embodiments of the present disclosure.

FIG. 2 illustrates an architecture comprising a set of systems for detecting, predicting, diagnosing, reporting on and treating oral conditions and/or oral health problems, in accordance with embodiments of the present disclosure.

FIG. 3 shows a block diagram of an example system for generating and/or displaying a dynamic tooth chart, in accordance with some embodiments of the present disclosure.

FIG. 4 illustrates a flow diagram of an example method for generating and/or displaying a dynamic tooth chart, in accordance with some embodiments of the present disclosure.

FIG. 5 illustrates a flow diagram of an example method for generating a tooth portion of a dynamic tooth chart, in accordance with some embodiments of the present disclosure.

FIG. 6 illustrates an example visualization of a dynamic tooth chart, in accordance with some embodiments of the present disclosure.

FIGS. 7A-B illustrates an example visualization of a dynamic tooth chart, in accordance with some embodiments of the present disclosure.

FIGS. 8A-C illustrate examples of visualizations of a dynamic tooth chart using various imaging modalities, in accordance with some embodiments of the present disclosure.

FIG. 9 illustrates an example UI displaying the visualization of the patient's dental arches in multiple imaging modalities simultaneously, in accordance with some embodiments of the present disclosure.

FIG. 10 illustrates an example UI portion displaying one or more visualizations of the patient's dental arches in multiple imaging modalities displayed in conjunction with two photographs of the patient, in accordance with some embodiments of the present disclosure.

FIG. 11 illustrates an example visualization of a dental chart in which periodontal data is projected onto an imaging modality, in accordance with embodiments of the present disclosure.

FIG. 12 illustrates a flow diagram of an example data flow for an automated dental charting system, in accordance with some embodiments of the present disclosure.

FIG. 13 illustrates a flow diagram of an example method for automatically generating tooth annotations for a dental chart, in accordance with some embodiments of the present disclosure.

FIG. 14 illustrates an example of automatically generated chart annotations displayed in an example dental chart, in accordance with some embodiments of the present disclosure.

FIG. 15 illustrates an example 2D color image and NIRI image depicting a healthy tooth, in accordance with some embodiments of the present disclosure.

FIG. 16 illustrates an example 2D color image and NIRI image depicting a crown on a tooth, in accordance with some embodiments of the present disclosure.

FIG. 17 illustrates an example 2D color image and NIRI image depicting a metal filling on a tooth, in accordance with some embodiments of the present disclosure.

FIG. 18 illustrates an example 2D color image and NIRI image depicting a composite filling on a tooth, in accordance with some embodiments of the present disclosure.

FIG. 19 illustrates an example 2D color image and NIRI image depicting a composite buccal filling on a tooth, in accordance with some embodiments of the present disclosure.

FIG. 20 illustrates an example 2D color image and NIRI image depicting the presence of an aligner attachment on a tooth, in accordance with some embodiments of the present disclosure.

FIG. 21 illustrates a block diagram of an example computing device, in accordance with some embodiments of the present disclosure.

### DETAILED DESCRIPTION

Described herein are embodiments for generating and displaying a dynamic tooth chart which can include automatically generated tooth annotations. The current standard in dentistry is to use a generic tooth chart to record and view dental and periodontal findings for a patient. However, there is little consistency in the visualization and arrangement of the teeth within the chart. For example, clinical findings can be indicated in the tooth chart using a variety of shapes and symbols, but there is no universal method for charting the different findings. Additionally, the shapes and symbols do not accurately reflect the actual condition of the patient's teeth. Examples of clinical findings can include existing conditions/restorations, planned treatment, watch, abnormalities, periodontal, pathology/disease, lab/medication prescriptions, medical history, notes, and so on. This lack of consistency results in the need for a legend to enable doctors, specialist, and/or third-party payors to use the tooth chart to relay patient information in an efficient and effective manner.

In dental practice, creating and maintaining accurate dental charts for each patient can be a time-consuming and error-prone process that typically relies on manual examination and documentation by a dental practitioner. This manual approach can be inefficient, particularly given the complexity of some dental conditions, the variety of restorative materials, and the need to track changes over time. A dental visit can involve a dental practitioner performing an evaluation, taking records (including dental and periodontal charting), making a diagnosis, and/or creating a treatment plan for the required dental work. Doctors and their teams may have different needs for patient data depending on the focus or reason for the visit. For example, one may need to record or see different levels of data for diagnostic purposes versus treatment purposes.

Some dental practitioners may use a practice management system (PMS) for managing patient records, which can include a digitization of the paper tooth chart. However, PMS platforms and digitized dental charts face numerous challenges that hinder their effectiveness and usability, including, for example, information overload, lack of standardization, and reconciling conflicting data. The amount of data a dental practitioner considers when making a diagnosis is ever increasing, and practitioners are often presented with excessive data that can be difficult to sift through and prioritize. Additionally, interpreting and integrating data from multiple imaging modalities, such as 3D scans, color images, near-infrared images, to provide a comprehensive and reliability assessment of dental status can be challenging. This can slow down the charting process, as well as increase the risk of overlooking critical details essential for patient care. Additionally, the presentation of the information is often static and is not tailored to the individual patient or doctor's current needs. These limitations hinder the efficiency, accuracy, and consistency of dental diagnostics and treatment planning.

Another concern with conventional dental charting systems is the lack of standardization across different systems and practices. Dental practitioners can use a variety of tooth chart formats, symbols, shading, and/or color to represent various clinical findings. The tooth chart's generalized visualization can result in complex, complicated, and inefficient workflows that make it difficult for a dental practitioner to find, understand, and analyze the available information for a patient. Without a universally accepted format, dental practitioners often encounter inconsistencies in notation and data presentation, making it challenging to interpret charts accurately. This fragmentation comprises the continuity of patient care and can lead to errors in diagnosis or treatment planning.

Usability is another persistent pain point. Many existing systems are not intuitive, requiring extensive training or cumbersome workarounds to input and retrieve information. This inefficiency can detract the time that practitioners could otherwise spend focusing on patient care. Additionally, these systems often rely on generic templates that fail to capture the nuanced and specific needs of diverse dental cases, further limiting their clinical utility. Another challenge arises when there is conflicting data among various imaging modalities, where a dental practitioner is to prioritize the data and exclude irrelevant information. Inaccurate and/or conflicting data can create confusion and undermine the credibility of dental records.

Accordingly, aspects and implementations of the present disclosure address the above-noted and other challenges by providing systems and method for generating and/or displaying a dynamic tooth chart, which can include automatically generated tooth annotations. In some embodiments, an oral health diagnostics system can include a dynamic tooth charting system and an auto charting system, as described throughout. In some embodiments, the dynamic tooth charting system can generate and display visualizations of a dental chart that dynamically change based on context, such as a patient's chief complaint, a treatment plan, and/or a scheduled service. The dynamic tooth charting system can serve as a visualization platform that enables viewing of dental records across multiple imaging modalities, normalizes dental records for consistent presentation, and/or provides interactive tools for switching between imaging modalities, adjusting overlay transparency, focusing on particular sections of the dental arch, and comparing historical data with current data. In some embodiments, the auto charting system can automatically generate tooth annotations by analyzing multi-modal intraoral scan data, such as 3D dental models, 2D color images, and NIRI images, e.g., using machine learning models and data processing techniques. The auto charting system can perform shape analysis, teeth arrangement analysis, and/or material classification to detect dental features such as restorations, dental accessories, missing teeth, and/or irregular tooth positioning, in embodiments. The output can include chart annotations identifying tooth status, positioning, and material composition.

In some embodiments, the auto charting system and the dynamic tooth charting system can be combined to provide an integrated dental charting solution. The auto charting system can process scan data to automatically generate chart annotations, including tooth identifications, restoration classifications, and/or arrangement indicators. The automatically generated annotations can be provided to the dynamic tooth charting system for inclusion in the visualization of the dental chart. The dynamic tooth charting system can display the auto-generated annotations overlaid on the patient's imaging data across multiple modalities. A dental practitioner can interact with the combined system to review the automatically generated annotations, make manual adjustments or corrections as needed, and/or switch between imaging modalities to verify findings. The combined system can provide both automated analysis capabilities and flexible visualization tools within a single integrated platform.

In some embodiments, the dynamic tooth charting system described herein can dynamically change the visualization of a dental chart (sometimes referred to herein as a dynamic tooth chart), e.g., based on a patient's chief complaint(s), a treatment or phased plan, and/or a scheduled service. For example, the dynamic tooth chart can automatically change from a comprehensive view to a more limited, problem-focused view (e.g., based on a use case). Additionally, or alternatively, the tooth chart can be changed between views manually by the user via a control panel. The dynamic tooth chart can expand its functionality as more data is provided (e.g., as more imaging is provided). In some embodiments, the dynamic tooth chart system enables visualization of historical data as compared with current data. In some embodiments, the dynamic tooth chart is not limited to teeth, and can include, for example, gums, bones, periodontal status, etc. For example, the dynamic tooth chart can include a hard tissue chart, a periodontal chart, and/or other charts.

In some embodiments, the dynamic tooth charting system can serve as a single platform for visualization of any imaging modality and record type for diagnostic record charting, treatment planning (e.g., including gathering and/or recording clinical findings on the actual image modality), patient education, evaluation of remotely monitored data, and/or simulation purposes. In some embodiments, the dynamic tooth charting system can serve as the data source for artificial intelligence (AI) assisted treatment decision support workflows (e.g., periodontal, restorative, and/or orthodontic). The dynamic tooth charting system can be used for all patient types, including primary, mixed (primary and permanent), and all permanent dentitions.

In some embodiments, the dynamic tooth charting system can provide a visualization of a tooth chart that enables one-to-many imaging modalities, where a mapping from one imaging modality allows an overlay to be projected onto any image type and displayed in the same layout. For example, radiograph data and AI detected clinical findings or treatment recommendations can be projected onto an intraoral scan, near-infrared, CBCT, photograph, video, and/or any other image modality.

In some embodiments, the dynamic tooth charting system can normalize dental records in terms of brightness, color, and/or orientation, with an option to disable for view of original state. In some embodiments, the dynamic tooth charting system can enable portions of the dental chart in various imaging modalities to automatically adjust to align to predetermined boundary. For example, the dynamic tooth charting system can automatically adjust the three-dimensional modality to match radiographic imaging (e.g., pan-ceph imaging). In some embodiments, the dynamic tooth charting system can enable a portion of the dynamic tooth chart to be swapped from one imaging modality to another (e.g., on demand by a user), while staying within the boundaries of the tooth chart visualization.

In some embodiments, the tooth chart visualization can include a portion designated as a dynamic record viewer that is context sensitive. In some embodiments, the dynamic record viewer can be configured to display information that is relevant to the current clinical context, such as the patient's chief complaint, the type of dental examination being performed, or the specific area of interest selected by the user (e.g., the dental practitioner). For example, when a dental practitioner selects a particular tooth or region within the chart, the dynamic record viewer can automatically present the corresponding imaging records, such as intraoral photographs, x-rays, and/or 3D scans, that illustrate the condition or finding associated with the selection. The content and/or layout of the dynamic record viewer can adapt in real-time (or near real-time) based on user interactions or predefined clinical workflows so that the dental practitioner is presented with pertinent data, avoiding having to manually search through disparate records.

In some embodiments, the dynamic tooth charting system can enable a user to correct for distortions, such as patient positioning, tube head angulation associated with an x-ray, jewelry artifacts associated with x-rays, etc. In some embodiments, the dynamic tooth charting system can provide automated and/or user-guided tools for identifying and compensating for such distortions within the displayed dental records. For example, image processing algorithms can detect and adjust for misalignments caused by improper patient positioning or tube head angulation, thereby improving the accuracy and consistency of the visualized data. In some embodiments, the dynamic tooth charting system can identify and mitigate the visual impacts of artifacts introduced by jewelry or other foreign objects, e.g., by filtering out the affected regions, and/or by alerting the user of their presence. By enabling these correction capabilities within the user interface, the dynamic tooth charting system can enable dental practitioners to review and interpret dental records with greater confidence and precision, thus reducing the likelihood of diagnostic errors attributed to image distortions.

In some embodiments, the dynamic tooth charting system can track information over time, thus enabling a dental practitioner to view the temporal charts in the dynamic tooth chart visualization. Since the dynamic tooth charting system can provide information about the patient's oral health over time, disease progression can be easily observed, thus facilitating making a treatment decision. In some embodiments, the dynamic tooth charting system can aggregate and display historical dental records, such as previous scans, x-rays, and/or clinical findings, alongside current data with the same charting interface. A dental practitioner can review changes in dental conditions, monitor disease progression, and/or assess the outcomes of prior treatment by navigating through different time points, and/or comparing side-by-side visualizations. In some embodiments, the dynamic tooth charting system can provide tools for highlighting differences and/or trends between time-separate records, which can help facilitate early detection of issues and support more informed clinical decision-making.

In some embodiments, the dynamic tooth charting system can generate and display a visualization of a tooth chart that displays patient data using images by recreating the most common type of dental chart using the patient's imaging. In some embodiments, a scan of maxillary (or upper) teeth and a scan of the mandibular (or lower) teeth can be converted into a linear form to display the buccal and/or lingual surfaces, with occlusal images in between each arch view.

In some embodiments, the dynamic tooth charting system can generate and display a visualization of a tooth chart that display all image modalities in a single account to mimic and merge multiple patterns currently used in dentistry, such as panoramic x-ray and the tooth chart. For example, the dynamic tooth charting system can display an intraoral scan, a CBCT, or a near-infrared of the upper jaw in a linear appearance and an intraoral scan, a CBCT, or a near-infrared of the lower jaw in a slight U-shaped curve, to resemble a panoramic x-ray. In a portion of the user interface, the dynamic tooth charting system can display an occlusal view of both arches to show all surfaces of the teeth simultaneously in a pattern that is familiar to dental practitioners. In some embodiments, the dynamic tooth charting system can provide controls to enable a user to layer the different types of images, thus enabling the user to view multiple image modalities in one visualization with adjustment tools for transparency, hide/show, and/or a slider to reveal layers not initially displayed. The overall focus of the visualization can be based on a variety of frameworks, such as, evaluation type, treatment type, and/or a patient's chief complaint(s).

In some embodiments, the dynamic tooth charting system can generate and display a visualization in which the teeth are unraveled and positioned in a standard tooth chart grid layout. A user may toggle between the various modalities. Such embodiments can facilitate orthodontic treatment planning (e.g., interproximal reduction (IPR) planning). In traditional orthodontia, a doctor might use a tooth chart as a reference and record of where to perform, and/or the amount performed, of IPR with no regards to the individual's' clinical condition (e.g., amount of enamel). This can lead to an underperformance of IPR, leading to unexpressed tooth movements and resulting in additional orthodontic treatment (e.g., additional aligners). The dynamic tooth charting system can provide the user the ability to visualize different layers of the tooth, showing the patient that IPR involves a negligible amount of reduction, and/or track the amount of reduction over time.

In some embodiments, the dynamic tooth charting system can enable or facilitate charting. In some embodiments, the dynamic tooth charting system can enable periodontal charting. The dynamic tooth charting system can project periodontal data, such as periodontal probings, gingival recession, bleeding points, furcation movement, gingival defects, mobility, mucogingival junction, etc., onto any imaging modality. In some embodiments, the dynamic tooth charting system can include indications of occlusion data, such as bruxism, occlusal trauma, excursive movements (canine guidance, lateral an protrusive movements), etc.

In some embodiments, the dynamic tooth charting system can enable patient education, e.g., by providing treatment plan presentations and obtaining informed consent. For example, the visualizations generated by the dynamic tooth charting system can enable patient education by simplifying complex concepts into easy-to-understand visualizations. In some embodiments, the dynamic tooth charting system can provide phased treatment presentation or report with explanations and interactive educations educational components. In some embodiments, the dynamic tooth charting system can provide a shareable dashboard for the patient to understand their oral health score and education to change their behavior. In some embodiments, the dashboard can include gamification components to influence behavioral changes. In some embodiments, the dynamic tooth charting system can include simulations, e.g., by projecting clinical findings (e.g., AI-detected clinical findings) in-face photos and/or videos, to help a patient understand their oral health status. In some embodiments, the dynamic tooth charting system can include treatment plans, e.g., to provide third-party payors a summary of clinical findings planned and/or completed treatment for pre-estimates/billing, optionally including a list of probabilities of success for each treatment option.

In some embodiments, the auto charting system described herein can leverage multi-model intraoral scan data (e.g., 3D dental models, color images, NIRI images) to identify, classify, and/or document the condition of each tooth and/or related dental structures. In some embodiments, the auto charting system can integrate data processing techniques, machine learning, and/or cross-validation techniques to detect dental features and restorations, as well as assess teeth arrangement and changes over time. The resulting tooth annotations can be used to populate a dental chart. In some embodiments, the auto charting system can interface with the dynamic tooth charting system, e.g., to populate the visualization of the tooth chart with the automatically generated tooth annotations.

In some embodiments, the auto charting system can receive (or otherwise identify) scan data, which can include multi-model intraoral scan data, such as a 3D model of a patient's dentition, 2D color images, and/or NIRI images. The auto charting system can perform semantic segmentation and/or instance segmentation on the 3D model data to identify and isolate individual teeth and surrounding soft tissues. The segmentation can provide the foundation for subsequent analyses. The auto charting system can then perform multiple analyses on the segmented data (optionally in parallel), including a shape analysis, a material analysis, and/or a teeth arrangement analysis. The shape analysis can use the segmented 3D model data (and optionally the color and/or NIRI image data) to determine indications of tooth identity, optionally including distinguishing between natural, primary, and/or non-natural structures such as abutments, scan bodies, and dental accessories (e.g., brackets or attachments). The material analysis can utilize the 2D color and/or NIRI images, mapped to the segmented 3D model, to differentiate between natural tooth material and various restorative materials. The results of the material analysis can be the identification of crowns, bridges, veneers, inlays, onlays, overlays, and/or significant fillings. The teeth arrangement analysis can compare the spatial positioning of each tooth relative to a modeled jawline curve. The result of the teeth arrangement analysis can include the detection of missing teeth and/or irregular tooth placements. The results from these analyses can be aggregated to generate annotations for each tooth of a dental chart. The annotations can include multiple findings per tooth.

In some embodiments, the auto charting system can incorporate cross-validation techniques, such as symmetry checks between corresponding teeth on opposite sides of the jaw, and/or comparisons across multiple scans of the same patient. For example, if, as a part of a symmetry check, a tooth's classification or detected condition significantly deviates from its symmetric counterpart, the auto charting system can determine a potential error in segmentation, identification, and/or material analysis. Upon making such a determination, the auto charting system can implement a correction or review of the identified asymmetry. As another example, as part of a multi-scan comparison check, the auto charting system can compare scans of the same patient taken at different times to validate findings, e.g., by checking for consistency in tooth identification and tracking the progression of a condition across visits. The auto charting system can use multi-scan comparisons to confirm genuine tooth changes, e.g., due to restorations or tooth movements, and avoid false findings caused by imaging artifacts or algorithmic misclassifications, for example. Thus, the cross-validation techniques can be used to correct errors and/or validate findings.

In some embodiments, the final output of the auto charting system can be an automatically populated dental chart that can be reviewed and/or edited by dental practitioners, and/or integrated into dental practice management systems. In some embodiments, the auto charting system can populate a dental chart to include the automatically generated annotations, including tooth identity, whether it is natural or primary, the presence and/or type of restorations such as crowns, bridges, veneers, inlay, and/or fillings, and/or the presence of dental accessories such as attachments or brackets. The annotations can also indicate spatial arrangement, e.g., noting missing teeth or irregular positioning. The dental chart can be designed to be easily reviewed and/or edited by dental practitioners, e.g., by providing a user interface (UI) that display the dental chart including the annotations. For example, a dental practitioner can interact with the dental chart presented on the UI by selecting individual teeth or conditions, verifying the tooth classification(s), and/or making manual adjustments or additions as needed. As an illustrative example, if the auto charting system misidentified a tooth, missed a restoration, or failed to detect a specific clinical condition, the dental practitioner can directly edit the chart to correct the findings. In some embodiments, the UI can enable annotation, deletion, and/or reclassification of findings, as well as the addition of notes or comments for further clinical context. In some embodiments, the dental chart can be integrated into a dental practice management system, and can provide a reliable, up-to-date, and clinically meaningful record that supports diagnostics, treatment planning, and ongoing patient care.

Embodiments described herein provide for an improved method and apparatus for generating and/or displaying a dynamic tooth chart that is patient-friendly, time-efficient, and capable of providing consistent and accurate indicators of clinical findings, thereby enhancing the overall quality of dental care and patient experience. Embodiments described herein directed to the dynamic tooth charting system provide technical improvements over traditional tooth charting systems that directly impact computer resource consumption and system efficiency. By normalizing and aligning diverse patient records (e.g., including 2D and 3D scans, x-rays, photographs, and/or AI-generated insights) into a standardized, context-sensitive chart format, the dynamic tooth charting system can reduce the need for multiple, redundant data processing pipelines and disparate visualization modules. For example, rather than maintaining separate viewers and data structures for each imaging modality, aspects of the present disclosure utilize a unified data model and normalization process, allowing all records to be displayed and manipulated within a single, familiar user interface. This consolidation can reduce memory usage and computational overhead, as they dynamic tooth charting system can reuse normalized data blocks across different views and clinical contexts without reprocessing the underlying images each time. Furthermore, the prioritization and context-sensitive selection of image modalities enables the most relevant data to be loaded and rendered for a given clinical scenario, minimizing unnecessary data retrieval and graphical processing. The ability to dynamically update the chart as new records are added, rather than reloading or recalculating the entire dataset, further streamlines resource utilization. Collectively, these technical features result in faster response times, lower GPU and/or GPU demands, and more scalable performance, particularly in environments where large volumes of multimodal dental data is accessed and visualized.

Embodiments described herein directed to the auto charting system provide technical improvements over traditional dental charting and diagnostics methods, such as computational efficiency and resource utilization. By utilizing multi-modal intraoral scan data, the methods and systems described herein enable a more comprehensive and precise analysis of dental structures and conditions than previously possible with manual charting or x-ray based automation. By employing multi-modal approach that integrates 3D models, color images, and NIRI images, the methods and systems described herein enable more targeted and context-aware processing, reducing the need for redundant and exhaustive analysis across all data types. The segmentation and ML models described herein help facilitate the accurate identification and classification of teeth, restorations, and/or dental accessories, while material analysis using NIRI and/or color imaging provides reliable differentiation between natural and artificial materials. Additionally, the use of segmentation and ML models allows for the extraction of relevant features and conditions in a streamlined, parallelized workflow, minimizing unnecessary data handling and computations. The integration of spatial arrangement analysis further enables the detection of missing teeth and/or irregular positioning, enhancing the completeness of dental records. Additionally, cross-validation techniques (e.g., symmetry checks and multi-scan comparisons) improve the robustness and reliability of the results by automatically correcting errors and validating findings. The cross-validation techniques further enhance efficiency by providing automated error correction and validation, reducing the need for repeated manual review or reprocessing. These technical advancements streamline the charting process, reduce clinician workload, minimize the risk of human error, and provide a more actionable data for diagnostics and treatment planning, resulting in faster processing times, lower memory and computational demands, and a scalable solution that can be deployed efficiently in clinical environments with limited hardware resources. Additionally, the automated charting data can be leveraged to refine other dental algorithms, such as caries detection and time-lapse analysis, further elevating the standard of care and efficiency in dental practice.

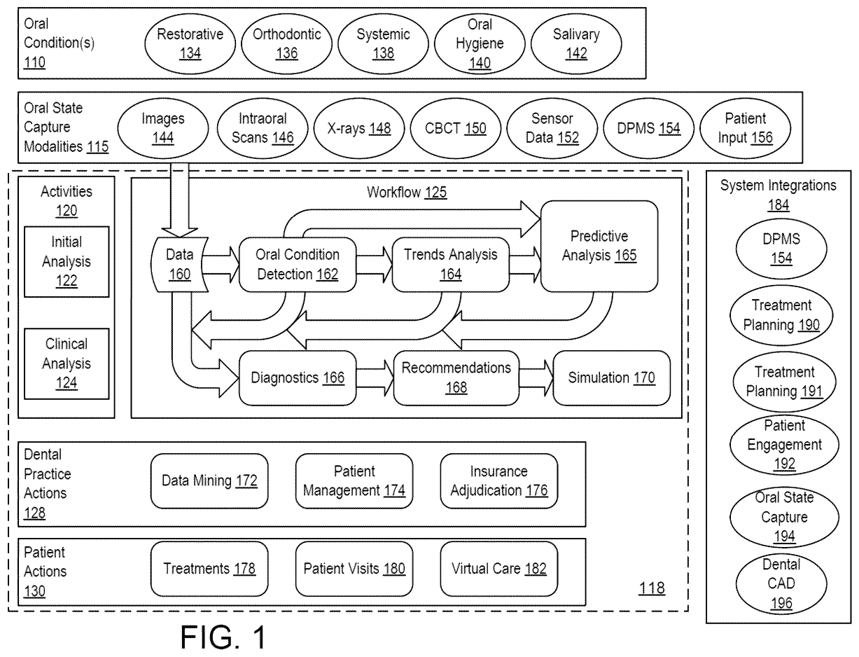

FIG. 1 illustrates a workflow 125 for detecting, predicting, diagnosing, and reporting on oral conditions (e.g., oral health conditions) by an oral health diagnostics system 118, in accordance with embodiments of the present disclosure. The workflow 125 may be a general digital workflow covering use of radiographs and/or other oral state capture modalities within a digital platform of integrated products/services to provide identifications of oral conditions and/or actionable symptom recommendations and/or diagnoses of oral health problems associated with such oral conditions. The workflow 125 may be used to assist doctors and/or users of an oral health diagnostics system 118 to assess a patient's oral health, identify oral conditions, diagnose dental health problems, provide actionable symptom recommendations, provide treatment recommendations, and so on. The workflow 125 may be executed by a digital platform of integrated products that provide dental condition identifications, actionable symptom recommendations and/or diagnoses of oral health problems using analysis of data from one or more oral state capture modalities, including radiographs (e.g., generated by radiography machines).

A patient may have one or more oral conditions 110. Oral conditions 110 may include or be related to caries, gum recession, gingival swelling, tooth wear, bleeding, malocclusion, tooth crowding, tooth spacing, plaque, tooth stains, and/or tooth cracks, for example. In some embodiments, the oral conditions 110 may include restorative conditions 134, orthodontic conditions 136, systematic conditions 138, oral hygiene conditions 140, salivary conditions 142, and so on. Restorative conditions 134 may include conditions such as caries that are addressable by performing restorative dental treatment. Such restorative dental treatment may include drilling and filling caries, performing root canals, forming preparations of teeth and applying caps or crowns to the preparations, pulling teeth, adding bridges to teeth, and so on. Restorative conditions may also include results of past restorative treatments of the patient's oral cavity. Examples of past restorations include fillings, caps, crowns, bridges, and so on. Orthodontic conditions may include conditions treatable via orthodontic treatment. Such orthodontic conditions may include a malocclusion (e.g., tooth crowding, overbite, underbite, posterior crossbite, posterior open bite, tooth gaps, etc.). Orthodontic conditions may be associated with restorative conditions in some instances. For example, tooth crowding may cause caries, which results in restorative treatment. Systematic conditions 138 may include conditions such as periodontitis, periodontal bone loss, gum recession, tooth wear, and so on. Systematic conditions 138 may be associated with restorative conditions 134 and/or orthodontic conditions 136. Oral hygiene conditions 140 may include brushing and flossing related conditions, such as development of calculus on teeth, caries, and so on. Oral hygiene conditions 140 may be related to restorative conditions 134, orthodontic conditions 136 and/or systematic conditions 138 in embodiments. Salivary conditions 142 may include a pH level of a patient's mouth that is outside of normal, a low level of saliva, and so on. Salivary conditions 142 may be related to restorative conditions 134, orthodontic conditions 136, systematic conditions 138 and/or oral hygiene conditions 140 in embodiments. For example, the detection and identification of salivary conditions may be used as an input to an ML model that can use such information to assess periodontal disease, acid reflux, vomiting, poor diet, oral cancer, and/or oropharyngeal cancer. For example, biomarkers of saliva may be used to assist in the assessment and/or management of periodontal disease. Tooth erosion, caries and/or saliva biomarkers may be used to identify acid reflux, vomiting and/or poor diet. In some instances, an oral condition of a patient may include a cross-classification. Such oral conditions may belong to multiple different categories of oral conditions 110. For example, caries may be a restorative condition 134, an orthodontic condition 136 and an oral hygiene condition 140.

A patient may have one or more oral health problems that may be root problems for the oral conditions and/or that may be caused by the oral conditions. In some embodiments, an oral condition also constitutes an oral health problem. Examples of oral health problems include caries, periodontal disease, a tooth root issue, a cracked tooth, a broken tooth, oral cancer, a cause of bad breath, and/or a cause of a malocclusion.

A dental practice (e.g., a group practice or solo practice) may capture data about a patient's oral state using one or more oral state capture modalities 115. A common oral state capture modality used by dental practices are radiographs (i.e., x-rays) 148 generated by radiography machines. There are multiple different types of x-rays that a genal practice may capture of a patient's oral cavity, including bite-wing x-rays, panoramic x-rays and periapical x-rays.

A bite-wing x-ray is a type of dental radiograph used to detect dental caries (cavities) and monitor the health of teeth and supporting bone. During a bite-wing x-ray, the patient bites down on a small tab or wing-shaped device attached to the x-ray film or sensor. This helps keep the film or sensor in place while the x-ray is taken. An x-ray machine (also referred to as a radiography machine) is positioned outside the mouth to capture images of the upper and lower teeth on one side of the mouth at a time. Accordingly, a bite-wing x-ray includes upper and lower teeth of one side of a patient's mouth. In embodiments, bite-wing x-rays are useful for detecting cavities between teeth and for assessing the fit of dental fillings and crowns. Bite-wing x-rays may also be used to help in diagnosing gum disease and/or to monitor bone levels around the teeth in embodiments.

A periapical x-ray, also known as a periapical radiograph, is a type of dental x-ray that focuses on specific areas of the mouth, particularly individual teeth and the surrounding bone. During a periapical x-ray, the dentist or dental radiographer positions an x-ray machine so that it captures detailed images of one or more teeth from crown to root, as well as the surrounding bone structure and supporting tissues. Periapical x-rays may provide a comprehensive view of the entire tooth, including the root tip (apex) and the bone around the tooth's root. In embodiments, periapical x-rays may be used to help diagnose oral health problems such as tooth decay (caries), infections or abscesses at the root of a tooth, bone loss around a tooth due to periodontal (gum) disease, abnormalities in the root structure or surrounding bone, evaluation of dental trauma or injuries, and so on. Periapical x-rays may also be used to assist in assessment of the status of teeth prior to dental procedures such as root canal treatment or extraction.

A panoramic x-ray, also known as a panoramic radiograph or orthopantomogram (OPG), is a type of dental radiograph that provides a comprehensive view of the entire mouth, including the teeth, jaws, temporomandibular joints (TMJ), and surrounding structures in a single image. During a panoramic x-ray, the patient stands or sits in an upright position while an x-ray machine rotates around their head in a semi-circle. The x-ray machine captures a continuous image as it moves, creating a detailed panoramic view of the entire oral and maxillofacial region. In embodiments, a panoramic x-ray may be used to assist in evaluation of the development and position of teeth, including impacted teeth, assessing the health of the jawbone and surrounding structures, detecting cysts, tumors, or other abnormalities in the jaw or adjacent tissues, planning orthodontic treatment by assessing tooth alignment and development, evaluating the placement and condition of dental implants, and/or diagnosing temporomandibular joint (TMJ) disorders or other jaw-related issues.

Another oral state capture modality that is increasingly common in dental practices are intraoral scans 146, and three-dimensional (3D) models of dental arches (or portions thereof) based on such intraoral scans. Intraoral scans are produced by an intraoral scanning system that generally includes an intraoral scanner and a computing device connected to the intraoral scanner by a wired or wireless connection. The intraoral scanner is a handheld device equipped with one or more small cameras and/or optical sensors. The dentist or dental professional moves the intraoral scanner around the patient's mouth, capturing multiple 3D images or scans of the teeth and surrounding structures from various angles. As the intraoral scanner captures the images or scans, they may be processed and displayed on a computer screen in real-time or near real-time. The collected images or scans are stitched together to create a complete 3D digital model of the patient's teeth and oral cavity. This digital impression can be manipulated, analyzed, and shared electronically with dental laboratories or specialists as needed.

An intraoral scan application executing on the computing device of an intraoral scanning system may generate a 3D model (e.g., a virtual 3D model) of the upper and/or lower dental arches of the patient from received intraoral scan data (e.g., images/scans). To generate the 3D model(s) of the dental arches, the intraoral scan application may register and stitch together the intraoral scans generated from an intraoral scan session. In one embodiment, performing image registration includes capturing 3D data of various points of a surface in multiple intraoral scans, and registering the intraoral scans by computing transformations between the intraoral scans. The intraoral scans may then be integrated into a common reference frame by applying appropriate transformations to points of each registered intraoral scan.

In one embodiment, registration is performed for each pair of adjacent or overlapping intraoral scans. Registration algorithms may be carried out to register two adjacent intraoral scans for example, which essentially involves determination of the transformations which align one intraoral scan with the other. Registration may involve identifying multiple points in each intraoral scan (e.g., point clouds) of a pair of intraoral scans, surface fitting to the points of each intraoral scans, and using local searches around points to match points of the two adjacent intraoral scans. For example, the intraoral scan application may match points, edges, curvature features, spin-point features, etc. of one intraoral scan with the closest points, edges, curvature features, spin-point features, etc. interpolated on the surface of the other intraoral scan, and iteratively minimize the distance between matched points. Registration may be repeated for each adjacent and/or overlapping scans to obtain transformations (e.g., rotations around one to three axes and translations within one to three planes) to a common reference frame. Using the determined transformations, the intraoral scan application may integrate the multiple intraoral scans into a first 3D model of the lower dental arch and a second 3D model of the upper dental arch.

The intraoral scan data may further include one or more intraoral scans showing a relationship of the upper dental arch to the lower dental arch. These intraoral scans may be usable to determine a patient bite and/or to determine occlusal contact information for the patient. The patient bite may include determined relationships between teeth in the upper dental arch and teeth in the lower dental arch.

Oral state capture modalities 115 may additionally or alternatively include one or more types of images 144 (e.g., 2D and/or 3D images) of a patient's oral cavity. In addition to generating intraoral scans, intraoral scanning systems may additionally be used to generate color 2D images of a patient's oral cavity. These color 2D images may be registered to the intraoral scans generated by the intraoral scanning system, and may be used to add color information to 3D models of a patient's dental arches. Intraoral scanning systems may additionally or alternatively generate 2D near infrared (NIR) images, images generated using fluorescent imaging, images generated under particular wavelengths of light, and so on. Such image generation may be interleaved with 3D image or intraoral scan generation by an intraoral scanner.

Dental practices may additionally include cameras for generating 3D images of a patient's oral cavity and/or cameras for generating 2D images of a patient's oral cavity. Additionally, a patient may generate images of their own oral cavity using personal cameras, mobile devices (e.g., tablet computers or mobile phones), and so on. In some instances, patients may generate images of their oral cavity based on the instruction of an application or service such as a virtual dental care application or service. In some cases, images of a patient's oral cavity (e.g., those taken by a dental practitioner or by a patient themselves) may be taken while the patient wears a cheek retractor to retract the lips and cheeks of the patient and provide better access for dental imaging (i.e., for intraoral photography).

Some dental practices also use cone beam computed tomography (CBCT) 150 as an oral state capture modality 115. CBCT is a medical imaging technique that uses a cone-shaped X-ray beam to create detailed 3D images of the dental and maxillofacial structures. CBCT scanners may be specifically designed for imaging the head and neck region, including the teeth, jawbones, facial bones, and surrounding tissues. A CBCT machine emits a cone-shaped X-ray beam that rotates around the patient's head. A detector on the opposite side of the machine captures a sequence of X-ray images from different angles. The x-ray images are processed to reconstruct them into a detailed 3D volumetric dataset. This dataset provides a comprehensive view of the patient's oral anatomy in three dimensions. CBCT scans may facilitate accurate diagnosis of various dental and maxillofacial conditions, including impacted teeth, dental infections, bone abnormalities, and temporomandibular joint disorders. In embodiments, CBCT imaging may be used for various dental and maxillofacial applications, including implant planning, orthodontic treatment planning, endodontic evaluations, oral surgery, and periodontal assessments.

In some embodiments, oral state capture modality 115 can include fluorescence imaging. Fluorescence imaging can refer to an optical imaging technique in which a dental tissue or material is illuminated with excitation light to induce emission of fluorescence radiation. The emitted fluorescence is captured by an imaging sensor. For example, for fluorescence imaging, an intraoral scanner may emit light at one or more particular frequencies. The light at the one or more particular frequencies may cause illuminated teeth to fluoresce. Areas of the teeth having caries, calculus, etc. may exhibit a different fluorescence than healthy areas of teeth, for example. The fluorescence of the teeth may have different colors, which may be captured in a color image.

In some embodiments, oral state capture modality 115 can include optical coherence tomography (OCT). OCT imaging can refer to a non-invasive optical imaging modality that directs low-coherence light toward a dental tissue and detects back-reflected signals to generate depth-resolved images based on optical interference. The resulting image data can represent internal tissue morphology.

For image-based oral state capture modalities, multiple depictions and views of the oral cavity and internal structures can be captured (e.g., in radiographs, intraoral scans, etc.). Examples of views include occlusal views, buccal views, lingual views, proximal-distal views, panoramic views, periapical views, bitewings views, and so on.

Oral state capture modalities 115 may additionally or alternatively include sensor data 152 from one or more worn sensors. In some instances, a patient may be prescribed a compliance device (e.g., an electronic compliance indicator), an orthodontic aligner, a palatal expander, a sleep apnea device, a night guard, a retainer, or other dental appliance to be worn by the patient. Any such dental appliance may include one or more integrated sensors, which may include force sensors, pressure sensors, pH sensors, sensors for measuring saliva bacterial content, temperature sensors, contact sensors, bio sensors, and so on. Sensor data from the sensor(s) of a dental appliance worn by a patient may be reported to oral health diagnostics system 118 in embodiments. Additionally, or alternatively, a patient may wear one or more consumer health monitoring tools or fitness tracking devices, such as a watch, ring, etc. that includes sensors for tracking patient activity, heartbeat, blood pressure, electrical heart activity (e.g., generates an electrocardiogram), breathing, sleep patterns, body temperature, and so on. Data collected by such fitness tracking devices may also be reported to the oral health diagnostics system 118 in embodiments.

Oral state capture modalities 115 may additionally or alternatively include patient input 156. Patient input may include patient complaints of pain, numbness, bleeding, swelling, clicking, etc. in one more regions of their mouth. Patient input may further include input on overall health, such as information on underlying health conditions (e.g., diabetes, high blood pressure, etc.), on patient age, and so on. Such patient input may be captured and input into an oral health diagnostics system 118 in embodiments. For example, a doctor or patient may type up notes or annotations indicating the patient input, which may be ingested by the oral health diagnostics system 118 with other oral state capture modalities 115.

In some embodiments, an oral health diagnostics system 118 may include one or more system integrations 184 with external systems, which may or may not be dental related. Such system integrations 184 may be for data to be provided to the oral health diagnostics system 118 and/or for the oral health diagnostics system 118 to provide data to the other system(s).

Dental practices generally use a dental practice management system (DPMS) 154 for managing the dental practices. A DPMS 154 is a software solution designed to streamline and automate various administrative and clinical tasks within a dental practice. DPMS 154 are tailored for the needs of dental offices and help dentists and their staff manage patient information, appointments, billing, and other aspects of dental practice management efficiently. A DPMS 154 allows a dental practice to maintain comprehensive patient records, including demographic information, medical history, treatment plans, and clinical notes. The DPMS 154 provides a centralized database that enables dental staff to access patient information quickly and efficiently. DPMS 154 generally includes features for scheduling patient appointments, managing appointment calendars, and sending appointment reminders to patients. DPMS 154 provides tools for creating and managing treatment plans for patients, including digital charting of dental procedures, diagnoses, and treatment progress. This helps dentists and hygienists track patient care effectively and ensure continuity of treatment. DPMS 154 may help to automate billing processes, including generating invoices, processing payments, and managing insurance claims. It can also verify patient insurance coverage, estimate treatment costs, and submit claims electronically to insurance providers for faster reimbursement. DPMS 154 may generate financial reports and analytics to help dental practices track revenue, expenses, and profitability.

In embodiments, data from a DPMS 154 is used as one type of oral state capture modality 115. Oral health diagnostics system 118 may interface with a DPMS 154 to retrieve patient records for a patient, including past oral conditions of the patient, doctor notes, patient information (e.g., name, gender, age, address, etc.), and so on.

In addition to an ability to ingest data from a DPMS 154, oral health diagnostics system 118 in embodiments may be able to generate reports and/or other outputs that can be ingested by the DPMS 154. Accordingly, once the oral health diagnostics system 118 performs an assessment of a patient's oral conditions, oral health problems, treatment recommendations, etc., the oral health diagnostics system 118 may format such data into a format that can be understood by the DPMS 154. The oral health diagnostics system may then automatically add new data entries to the DPMS 154 for a patient based on an analysis of patient data from one or more oral state capture modalities 115.

The oral health diagnostics system 118 may have a system integration with one or more oral state capture systems (e.g., such as an intraoral scanner or intraoral scanning system) 194, from which intraoral scans 146, images 144, 3D models, and/or data from other oral state capture modalities may be received. Examples of oral state capture systems include an intraoral scanning system, a radiograph system or machine, a CBCT machine, and so on.

In embodiments, an output of oral health diagnostics system 118 may be provided to a dental computer aided drafting (CAD) system 196, such as Exocad® by Align Technology. The dental CAD system 196 may be used for designing dental restorations such as crowns, bridges, inlays, onlays, veneers, and dental implant restorations. The dental CAD system 196 may provide a comprehensive suite of tools and features that enable dental professionals to create precise and customized dental restorations digitally. The dental CAD system 196 may import digital impressions (e.g., 3D digital models of a patient's dental arches) captured using intraoral scanners, and may further import data on a patient's oral health from oral health diagnostics system 118. For example, the oral health diagnostics system 118 may export a report on a patient's oral health to the dental CAD system 196, which may be used together with a digital impression of the patient's dental arches to develop an appropriate restoration for the patient, for implant planning, for planning of surgery for implant placement, and so on.

In embodiments, oral health diagnostics system 118 may have a system integration 184 with a patient engagement system (e.g., which may include a patient portal and/or patient application) 192. The patient portal may be a portal to an online patient-oriented service. Similarly, the patient application may be an application (e.g., on a patient's mobile device, tablet computer, laptop computer, desktop computer, etc.) that interfaces with a patient-oriented service.

In an example, oral health diagnostics system 118 may integrate with a virtual care system. The virtual care system may provide a suite of digital tools and services designed to enhance patient care and communication between orthodontists/dentists and their patients. The virtual care system may leverage technology to facilitate remote monitoring, consultation, and treatment planning, allowing patients to receive dental care more conveniently and effectively.

In one embodiment, the patient engagement system 192 is or includes a virtual care system that may provide remote monitoring, teleconsultation, treatment planning, patient education and engagement, data management, and data analytics. With respect to remote monitoring, the virtual care system enables orthodontists and dentists to remotely monitor their patients'treatment progress (e.g., for orthodontic treatment) using advanced digital tools. This may include the use of smartphone apps, patient portals, or other software platforms that allow patients to capture and upload photos or videos of their teeth and orthodontic appliances. Such patient uploaded data may be provided to oral health diagnostics system 118 for automated assessment in embodiments. With regards to patient education and engagement, the virtual care system may provide reports, presentations, etc. generated by oral health diagnostics system 118 to patients (e.g., via a patient portal and/or application). For example, the dental health diagnostics system 118 may automatically generate informational videos, treatment progress trackers, compliance reminders, reports, presentations, and so on that are tailored to a patient's oral health, which may be provided to the patient via the patient portal and/or application.

In embodiments, oral health diagnostics system 118 may have a system integration 184 with one or more treatment planning system 190 and/or treatment management system 191 such as ClinCheck® provided by Align Technology®. For example, oral health diagnostics system 118 may have a system integration with an orthodontic treatment planning system and/or with a restorative dental treatment planning system. A treatment planning system 190 may use digital impressions and/or a report output by oral health diagnostics system 118 to plan an orthodontic treatment and/or a restorative treatment (e.g., to plan an ortho-restorative treatment). The treatment planning system 190 may plan and simulate orthodontic and/or restorative treatments. Treatment management system 191 may then receive data during treatment and determine updates to the treatment based on the treatment plan and the updated data.

In an example, an orthodontic treatment planning system may use advanced 3D imaging technology to create virtual models of patients'teeth and jaws based on digital impressions or intraoral scans. These digital models may be used to plan and simulate the entire course of orthodontic treatment, including the movement of individual teeth and the progression of treatment over time. Orthodontists can specify the desired tooth movements, treatment duration, and other parameters, taking into account a report provided by oral health diagnostics system 118, to create personalized treatment plans tailored to each patient's unique anatomy, oral health, and preferences. The orthodontic treatment planning system enables orthodontists to simulate the step-by-step progression of orthodontic treatment virtually, showing patients how their teeth will gradually move and align over the course of treatment. Orthodontists can visualize the planned tooth movements in 3D and make adjustments as needed to optimize treatment outcomes. The orthodontic treatment planning system may provide orthodontists and patients with visualizations of the predicted treatment outcomes, including before-and-after simulations that demonstrate the expected changes in tooth position and alignment, and how those changes might affect the patient's overall oral health as optionally predicted by the oral health diagnostics system 118. These visualizations help patients understand the proposed treatment plan and make informed decisions about their orthodontic care.

During treatment, updated data may be gathered about a patient's dentition, and such data (e.g., in the form of one or more oral state capture modalities 115) may be processed by the oral health diagnostics system 118, optionally in view of an already generated orthodontic treatment plan, to generate an updated report of the patient's overall oral health. The updated report may be provided by the oral health diagnostics system 118 to the orthodontic treatment planning system and/or orthodontic treatment management system to enable the orthodontic treatment planning/management system to perform informed modifications to the treatment plan. Thus, integration of the oral health diagnostics system with the orthodontic treatment planning system and/or treatment management system supports an iterative design process, allowing orthodontists to review and refine treatment plans based on patient feedback, clinical considerations, treatment progress, and automated reports output by oral health diagnostics system 118. This enables orthodontists to make adjustments to the treatment plan within the orthodontic treatment planning system and/or treatment management system and generate updated simulations to assess the impact of these changes on the final treatment outcome.

Accordingly, oral health diagnostics system 118 may perform treatment planning and/or management on its own and/or based on integration with one or more treatment planning systems for planning and/or managing orthodontic treatment, restorative treatment, and/or ortho-restorative treatment. An output of such planning may be an orthodontic treatment plan, a restorative treatment plan, and/or an ortho-restorative treatment plan. A doctor may provide one or more modifications to the generated treatment plan, and the treatment plan may be updated based on the doctor modifications.

In addition to those systems mentioned herein that oral health diagnostics system 118 may integrate with, oral health diagnostics system 118 may integrate with any system, application, etc. related to dentistry and/or orthodontics.

Oral health diagnostics system 118 may execute a workflow 125 that includes processing and analysis of data 160 from one or more oral state capture modalities 115. The workflow 125 may be roughly divided into activities 120 associated with an initial analysis 122 of a patient's oral health and operations associated with a clinical analysis 124 of the patient's oral health in some embodiments. One of more of the operations of the workflow may be performed by and/or assisted by application of artificial intelligence and/or machine learning models in embodiments. Multiple embodiments are discussed with reference to machine learning models herein. It should be understood that such embodiments may also implement other artificial intelligence systems or models, such as large language models in addition to or instead of traditional machine learning models such as artificial neural networks.

The workflow may include performing oral condition detection at block 162. To perform oral condition detection, a segmentation pipeline may process the data 160 to segment the data into one or more teeth and into one or more oral conditions that may be associated with the one or more of the teeth. The segmentation pipeline may include multiple different trained machine learning models and additional logic that operate on the data and/or on outputs of other trained machine learning models and/or logic to identify specific teeth and apply tooth numbering to the teeth, identify oral conditions, associate the oral conditions to specific teeth, determine locations on the teeth at which the oral conditions are identified, and so on. The output of block 162 may include masks indicating pixels of input image data (e.g., radiographs, 3D models, 2D images, etc.) associated with particular dental conditions, indications of which teeth have detected oral conditions, masks indicating, for each tooth in the input data, which pixels represent that tooth, and so on. The output of block 162 may be input into one or more of block 164, block 165 and/or block 166 in embodiments.

At block 164, trends analysis may be performed based on the output of block 162 and on prior oral conditions of the patient detected at one or more previous times. Trends analysis may include comparing oral conditions at one or more previous times to current oral conditions of the patient. Based on the comparison, an amount of change of one or more of the oral conditions may be determined, a rate of change of the one or more oral conditions may be determined, and so on. Trends analysis may be performed using traditional image processing and image comparison. Additionally, or alternatively, trends analysis may be performed by inputting current and past oral conditions and/or data from one or more oral state capture modalities into one or more trained machine learning models. An output of block 164 may be provided to block 165 and/or block 166 in embodiments.

At block 165, predictive analysis may be performed on the output of block 162, on the output of block 164 and/or on prior oral conditions of the patient detected at one or more previous times. Predictive analysis may include predicting future oral conditions of the patient based on input data. Predictive analysis may be performed with or without an input of prior oral conditions. If prior oral conditions are used in addition to current oral conditions to predict future conditions, then the accuracy of the prediction may be increased in embodiments. In some embodiments, predictive analysis is performed by projecting identified trends determined from the trends analysis into the future. In some embodiments, predictive analysis is performed by inputting the current and/or past oral conditions into one or more trained machine learning models that output predictions of future dental conditions. Predictive analysis may be performed using traditional image processing and image comparison. Additionally, or alternatively, predictive analysis may be performed by inputting current and/or past oral conditions, trends and/or data from one or more oral state capture modalities into one or more trained machine learning models. In embodiments, the predictive analysis generates synthetic image data, which may include panoramic views, periapical views, bitewing views, buccal views, lingual views, occlusal views, and so on of the predicted future oral conditions. Generated synthetic image data may be in the form of synthetic radiographs, synthetic color images, synthetic 3D models, and so on. An output of block 165 may be provided to block 166 in embodiments.

At block 166, automated diagnostics of a patient's oral health may be performed based on data 160 and/or based on outputs of block 162, block 164 and/or block 165 in embodiments. In embodiments, one or more trained machine learning (ML) models and/or artificial intelligence (AI) models may process input data to perform the diagnostics. An output of the ML models and/or AI models may include actionable symptom recommendations usable to diagnose oral health problems and/or actual diagnoses of oral health problems associate with the detected oral conditions.

At block 168, based on the data 160, oral conditions identified at block 162, output of trends analysis performed at block 164, output of predictive analysis performed at block 165 and/or output of diagnostics performed at block 166, processing logic may generate one or more treatment recommendations for a patient. The treatment recommendations may include multiple different treatment options, with different probabilities of success associated with the different treatment options.

At block 170, processing logic may generate one or more treatment simulations based on one or more of the treatment recommendations. The treatment simulations may include an alternative predictive analysis that shows predicted states of oral conditions and/or oral health problems of the patient after treatment is performed, or after one or more stages of treatment are performed. Treatment simulations may include generated synthetic image data, which may be in the form of synthetic radiographs, synthetic color images, synthetic 3D models, and so on. The synthetic image data may show what a patient's oral cavity would look like after treatment and/or after one or more intermediate and/or final stages of a multi-stage treatment (e.g., such as orthodontic treatment or ortho-restorative treatment).

Post treatment simulations may be compared to predicted simulations of the predicted states of the oral conditions absent treatment (e.g., as determined at block 165) in embodiments.

In embodiments, a report may be generated including the data 160 and/or outputs of one or more of blocks 162, 164, 165, 166, 168 and/or 170. The report may include labeled images, a dental chart, notes, annotations, and/or other information. The report may include a dynamic presentation (e.g., a video) that shows progression of dental conditions over time in some embodiments. The report may be stored in a data store and/or exported to one or more other systems (e.g., DPMS 154, treatment planning system 190, patient engagement system 192, dental CAD system 196).

The oral health diagnostics system 128 may perform multiple dental practice actions 128 and/or patient actions 130 in addition to, or instead of, storing a generated report and/or exporting the report to other systems. Examples of dental practice actions 128 that may be performed include data mining 172, patient management 174 and/or insurance adjudication 176. Examples of patient actions 128 that may be performed include treatments 178, patient visits 180 and/or virtual care 182. One or more of the actions may be performed based on leveraging external systems in embodiments. For example, virtual care 182 may be performed based on leveraging a patient portal and/or application of a virtual dental care system. Patient visits 180 may be performed based on leveraging a DPMS 154. Treatments 178 may be performed based on leveraging a treatment planning system 190 for planning, tracking and/or management of a treatment. Patient management 174 and/or insurance adjudication 176 may be performed based on leverage of a DPMS 154.

Data mining 172 may include analysis of patient data of a dental practice in embodiments. Data mining may be performed for a single dental practice or for multiple different dental practices. Data mining may be performed to determine strengths and weaknesses of a dental practice relative to other dental practices and/or to determine strengths and weaknesses of individual doctors relative to other doctors within a dental practice and/or outside of a dental practice (e.g., in a geographic region). As a result of data mining 172, a report may be generated indicating things for a doctor to focus on, types of procedures that a doctor should perform more, oral state capture modalities that a doctor should use more frequently, and so on.

Patient management 174 for a dental practice may include a range of tasks and processes aimed at providing quality care and ensuring positive experiences for patients throughout their interactions with the dental practice. Patient management may include appointment scheduling, patient registration and check-in, medical and dental history and records management (e.g., including information about past treatments, allergies, medications, and relevant medical conditions for each patient), treatment planning and coordination, financial management and billing (e.g., including collecting payments, processing insurance claims, providing cost estimates, and discussing payment options or financing arrangements with patients), patient communication and education (e.g., providing information about treatments, procedures, and oral hygiene instructions, as well as addressing patient concerns, answering questions, and maintaining open lines of communication throughout the treatment process), follow-up and recall, and patient satisfaction and feedback management.

Insurance adjudication 176 for a dental practice refers to the process of evaluating and determining the coverage and reimbursement for dental services provided to patients by their dental insurance carriers. Insurance adjudication 176 involves submitting claims to insurance companies, reviewing the claims for accuracy and completeness, and processing them according to the terms of the patient's insurance policy. After providing dental services (e.g., treatment) to a patient, the dental practice submits a claim to the patient's insurance company electronically or via paper. The claim includes information such as the patient's demographic details, treatment provided, diagnosis codes, procedure codes (CPT or ADA codes), and any other relevant documentation. In embodiments, such documentation is automatically prepared by oral health diagnostics system 118. Upon receiving an insurance claim, the insurance company reviews the claim to determine coverage eligibility and benefits according to the terms of the patient's insurance policy. The insurance company evaluates the claim and calculates the amount of coverage and reimbursement based on the patient's benefits plan, contractual agreements with the dental office, and applicable fee schedules. The adjudication process may involve verifying the accuracy of the submitted information, applying deductibles, copayments, and coinsurance, and determining the allowed amount for each covered service. In embodiments, oral health diagnostics system 118 may automatically generate responses to inquiries from insurance companies about already submitted claims. After adjudicating a claim, the insurance company sends an Explanation of Benefits (EOB) to the dental office and the patient. The EOB outlines the details of the claim, including the services rendered, the amount covered by insurance, any patient responsibility (such as copayments or deductibles), and the reason for any denials or adjustments. If the claim is approved, the insurance company issues payment to the dental office for the covered services. The dental office then reconciles the payment received with the treatment provided and updates the patient's financial records accordingly. If there are any discrepancies or denials, the dental office may need to follow up with the insurance company to resolve issues or appeal denied claims. In embodiments, oral health diagnostics system 118 automatically handles such follow-ups. After insurance adjudication, the dental office bills the patient for any remaining balance or patient responsibility not covered by insurance, such as deductibles, copayments, or non-covered services. The patient is responsible for paying these amounts according to the terms of their insurance policy and the dental office's financial policies.

In some embodiments, the workflow 125 can be implemented with just a few clicks of a web portal or dental practice application to enable doctors to purchase and activate one or more oral health diagnostics services. When patient records (e.g., data from one or more oral state capture modalities 115, such as intraoral scans 146, virtual care images 144, digital x-rays 148, etc.) are collected as a routine part of a dental appointment, these records may be uploaded to a digital platform of the oral health diagnostics system 118. The oral health diagnostics system 118 may start an analysis for the different oral (e.g., clinical) conditions that have been activated for the patient by the doctor, and may generate a report on the different identified oral conditions. In seconds the doctor may receive a report that has visual indications with colored clues of assessments for a number of possible dental conditions, dental health problems, and so on. As an example, the oral health diagnostics system 118 can send this data to the treatment planning system 190 or treatment management system 191 to process.

In some embodiments, the treatment planning system 190 can integrate this information with an orthodontic treatment plan. The doctor can share the analysis visually chairside with the patient and provide treatment recommendations based on the diagnosis. This can occur on the treatment planning and/or management system 190, 191 or on an application on an intraoral scanning system or x-ray system, for example. The doctor can also share the analysis with the patient and send visual assessments via patient engagement system 192. Integrated education modules may provide interactive context sensitive education tools designed to help the doctor diagnose and help convert the patient to the treatment in embodiments.

Some of the analyses that are performed to assess the patient's dental health are oral health condition progression analyses that compare oral conditions of the patient at multiple different points in time. For example, one carries assessment analysis may include comparing caries at a first point in time and a second point in time to determine a change in severity of the caries between the two points in time, if any. Other time-based comparative analyses that may be performed include a time-based comparison of gum recession, a time-based comparison of tooth wear, a time-based comparison of tooth movement, a time-based comparison of tooth staining, and so on. In some embodiments, processing logic automatically selects data collected at different points in time to perform such time-based analyses. Alternatively, a user may manually select data from one or more points in time to use for performing such time-based analyses.

In one embodiment, the different types of oral conditions for which analyses are performed and that are included in the detected oral conditions include tooth cracks, gum recession, tooth wear, occlusal contacts, crowding and/or spacing of teeth and/or other malocclusions, plaque, tooth stains, calculus, bone loss, bridges, fillings, implants, crowns, impacted teeth, root-canal fillings, and caries. Additional, fewer and/or alternative oral conditions may also be analyzed and reported. In embodiments, multiple different types of analyses are performed to determine presence, location and/or severity of one or more of the oral conditions. One type of analysis that may be performed is a point-in-time analysis that identifies the presence and/or severity levels of one or more oral conditions at a particular point-in-time based on data generated at that point-in-time (e.g., at block 162). For example, a single x-ray image of a dental arch may be analyzed to determine whether, at a particular point-in-time, a patient's dental arch included any caries, gum recession, tooth wear, problem occlusion contacts, crowding, spacing or tooth gaps, plaque, tooth stains, and/or tooth cracks. Another type of analysis that may be performed is a time-based analysis that compares oral conditions at two or more points in time to determine changes in the oral conditions, progression of the oral conditions and/or rates of change of the oral conditions (e.g., at block 164). For example, in embodiments a comparative analysis is performed to determine differences between x-rays taken at different points in time. The differences may be measured to determine an amount of change, and the amount of change together with the times at which the intraoral scans that were used to generate the x-rays were taken may be used to determine a rate of change. This technique may be used, for example, to identify an amount of change and/or a rate of change for tooth wear, staining, plaque, crowding, spacing, gum recession, caries development, tooth cracks, and so on.

In embodiments, one or more trained models are used to perform at least some of the one or more oral condition analyses. The trained models may include physics models and/or machine learning models, for example. In one embodiment, a single model may be used to perform multiple different analyses (e.g., to identify any combination of tooth cracks, gum recession, tooth wear, occlusal contacts, crowding and/or spacing of teeth and/or other malocclusions, plaque, tooth stains, and/or caries). Additionally, or alternatively, different models may be used to identify different oral conditions. For example, a first model may be used to identify tooth cracks, a second model may be used to identify tooth wear, a third model may be used to identify gum recession, a fourth model may be used to identify problem occlusal contacts, a fifth model may be used to identify crowding and/or spacing of teeth and/or other malocclusions, a sixth model may be used to identify plaque, a sixth model may be used to identify tooth stains, and/or a seventh model may be used to identify caries.

In one embodiment, at block 162 intraoral data from one or more points in time are input into one or more trained machine learning models that have been trained to receive the intraoral data as an input and to output classifications of one or more types of oral conditions. In one embodiment, the trained machine learning model(s) is trained to identify areas of interest (AOIs) from the input intraoral data and to classify the AOIs based on oral conditions. The AOIs may be or include regions associated with particular oral conditions. The regions may include nearby or adjacent pixels or points that satisfy some criteria, for example. The intraoral data that is input into the one or more trained machine learning model may include three-dimensional (3D) data and/or two-dimensional (2D) data. The intraoral data may include, for example, one or more 3D models of a dental arch, one or more projections of one or more 3D models of a dental arch onto one or more planes (optionally comprising height maps), one or more x-rays of teeth, one or more CBCT scans, a panoramic x-ray, near-infrared and/or infrared imaging data, color image(s), ultraviolet imaging data, intraoral scans, one or more bitewing x-rays, one or more periapical x-rays, and so on. If data from multiple imaging modalities are used (e.g., panoramic x-rays, bitewing x-rays, periapical x-rays, CBCT scans, 3D scan data, color images, and NIRI imaging data), then the data may be registered and/or stitched together so that the data is in a common reference frame and objects in the data are correctly positioned and oriented relative to objects in other data. One or more feature vectors may be input into the trained model, where the feature vectors include multiple channels of information for each point or pixel of an image. The multiple channels of information may include color channel information from a color image, depth channel information from intraoral scan data, a 3D model or a projected 3D model, intensity channel information from an x-ray image, and so on.

The trained machine learning model(s) may output a probability map, where each point in the probability map corresponds to a point in the intraoral data (e.g., a pixel in an intraoral image or point on a 3D surface) and indicates probabilities that the point represents one or more dental classes. In one embodiment, a single model outputs probabilities associated with multiple different types of dental classes, which includes one or more oral health condition classes. In an example, a trained machine learning model may output a probability map with probability values for a teeth dental class and a gums dental class. The probability map may further include probability values for tooth cracks, gum recession, tooth wear, occlusal contacts, crowding and/or spacing of teeth and/or other malocclusions, plaque, tooth stains, healthy area (e.g., healthy tooth and/or healthy gum) and/or caries. In the case of a single machine learning model that can identify each of tooth cracks, gum recession, tooth wear, occlusal contacts, crowding and/or spacing of teeth and/or other malocclusions, plaque, tooth stains, and caries, eleven valued labels may be generated for each pixel, one for each of teeth, gums, healthy area, tooth cracks, gum recession, tooth wear, occlusal contacts, crowding and/or spacing of teeth and/or other malocclusions, plaque, tooth stains, and caries. The corresponding predictions have a probability nature: for each pixel there are multiple numbers that may sum up to 1.0 and can be interpreted as probabilities of the pixel to correspond to these classes. In one embodiment, the first two values for teeth and gums sum up to 1.0 and the remaining values for healthy area, tooth cracks, gum recession, tooth wear, occlusal contacts, crowding and/or spacing of teeth and/or other malocclusions, plaque, tooth stains, and/or caries sum up to 1.0.

In some instances, multiple machine learning models are used, where each machine learning model identifies a subset of the possible oral conditions. For example, a first trained machine learning model may be trained to output a probability map with three values, one each for healthy teeth, gums, and caries. Alternatively, the first trained machine learning model may be trained to output a probability map with two values, one each for healthy teeth and caries. A second trained machine learning model may be trained to output a probability map with three values (one each for healthy teeth, gums and tooth cracks) or two values (one each for healthy teeth and tooth cracks). One or more additional trained machine learning models may each be trained to output probability maps associated with identifying specific types of oral conditions.

In embodiments, image processing and/or 3D data processing may be performed on radiographs and/or other dental data. Such image processing and/or 3D data processing may be performed using one or more algorithms, which may be generic to multiple types of oral conditions or may be specific to particular oral conditions. For example, a trained model may identify regions on a dental radiograph that include caries, and image processing may be performed to assess the size and/or severity of the identified caries. The image processing may include performing automated measurements such as size measurements, distance measurements, amount of change measurements, rate of change measurements, ratios, percentages, and so on. Accordingly, the image processing and/or 3D data processing may be performed to determine severity levels of oral conditions identified by the trained model(s). Alternatively, the trained models may be trained both to classify regions as caries and to identify a severity and/or size of the caries.

The one or more trained machine learning models that are used to identify, classify and/or determine a severity level for oral conditions may be neural networks such as deep neural networks or convolutional neural networks. Such machine learning models may be trained using supervised training in embodiments.

A dentist, after a quick glance at the dental diagnostics summary, may determine that a patient has carries, clinically significant tooth wear, and crowding/spacing and/or other malocclusions and/or oral conditions.

In embodiments, the oral health diagnostics system, and in particular the dental diagnostics summary, helps a doctor to quickly detect oral conditions (e.g., oral health conditions) and/or oral health problems and their respective severity levels, helps the doctor to make better judgments about treatment of oral conditions and/or oral health problems, and further helps the doctor in communicating with a patient that patient's oral conditions and/or oral health problems and possible treatments. This makes the process of identifying, diagnosing, and treating oral conditions and/or oral health problems easier and more efficient. The doctor may select any of the oral conditions and/or oral health problems to determine prognosis of that condition as it exists in the present and how it will likely progress into the future. Additionally, the oral health diagnostics system may provide treatment simulations of how the oral conditions and/or oral health problems will be affected or eliminated by one or more treatments.

In embodiments, a doctor may customize the oral conditions, oral health problems and/or areas of interest by adding emphasis or notes to specific oral conditions, oral health problems and/or areas of interest. For example, a patient may complain of a particular tooth aching. The doctor may highlight that particular tooth on the radiograph. Oral conditions that are found that are associated with the particular highlighted or selected tooth may then be shown in the dental diagnostics summary. In a further example, a doctor may select a particular tooth (e.g., lower left molar), and the dental diagnostics summary may be updated by modifying the severity results to be specific for that selected tooth. For example, if for the selected tooth an issue was found for caries and a possible issue was found for tooth stains, then the dental diagnostics summary would be updated to show no issues found for tooth wear, occlusion, crowding/spacing, plaque, tooth cracks, and gum recession, to show a potential issue found for tooth stains and to show an issue found for caries. This may help a doctor to quickly identify possible root causes for the pain that the patient complained of for the specific tooth that was selected. The doctor may then select a different tooth to get a summary of dental issues for that other tooth.

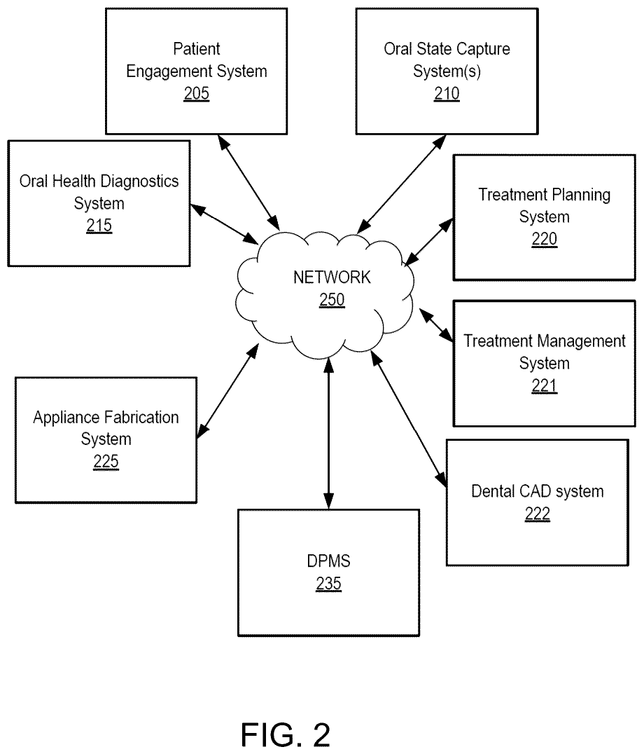

FIG. 2 illustrates an architecture comprising a set of systems for detecting, predicting, diagnosing, reporting on and treating oral conditions and/or oral health problems, in accordance with embodiments of the present disclosure. The systems in one embodiment include a patient engagement system 205, one or more oral state capture systems 210, a treatment planning and/or management system 220, a treatment management system 221, a dental CAD system 222, a DPMS 235, an appliance fabrication system 225, and/or an oral health diagnostics system 215.

In embodiments, oral health diagnostics system 215 corresponds to oral health diagnostics system 118 of FIG. 1.

Furthermore, in embodiments patient engagement system 205 corresponds to corresponds to patient engagement system 192.

In further embodiments, treatment planning system 220 may correspond to treatment planning system 190 and/or treatment management system 221 may correspond to treatment management system 191 of FIG. 1. The treatment planning and/or management systems 220, 221 may provide treatment plans, treatment recommendations, orthodontic/restorative integration capabilities, and/or other capabilities. These systems may, in some implementations, take in representations of dentition, identify (through human activities and/or automation) orthodontic/restorative treatments to dentition, provide staging/intermediate positioning/final positioning capabilities of orthodontic/restorative treatments, receive and/or process modifications to the treatment plan, provide updated treatments, support appliance design, etc. In some implementations, the treatment planning systems implement an end-to-end digital treatment planning workflow.

Treatment planning system 220 may provide controls for modifying and/or moving oral structures, teeth, etc. In some embodiments, treatment planning system 220 Includes hard limits on some movements, oral structure positions, etc., and may determine when and/or where certain types of interactions are permitted. This may include comparison of an instructed movement/position of one or more oral structures against entries in hard limit databases that store information about movements that are and/or not feasible.

The treatment management system 221 and/or oral health diagnostics system 215 may provide interactive tools to allow users to plan/manage treatments, examine the state of a person's dentition, evaluate data from x-rays and various oral state capture modalities, and so on. The treatment management system 221 and/or oral health diagnostics system 215 can provide users with an immersive experience where they can evaluate and/or annotate a person's dentition, plan/implement possible treatments for the person's dentition, review/approve/implement actions/recommendations, etc. In some implementations, the treatment management system 221 and/or oral health diagnostics system 215 implements one or more standalone tools related to interaction with a segmented radiographic representation of the oral cavity. The oral health diagnostics system 215 may communicate to the treatment planning system 220 and/or treatment management system 221 for ortho-restorative capabilities through APIs or other architecture.

In some implementations, the oral health diagnostics system 215 is combined with ortho-restorative capabilities (e.g., e.g., treatment planning and/or treatment management functionalities). In such implementations, a user might be presented with ortho-restorative capabilities on one or more 3D models of a dental arch (e.g., generated from an intraoral scan scan) of the oral cavity. The 3D model(s) may show teeth represented from an intraoral scan, and may show teeth and other oral structures and/or conditions as determined from a segmented radiographic representation of the oral cavity. In these examples, aspects of the segmented radiographic representation of the oral cavity and the depiction of the oral cavity from the intraoral scan may be overlaid, blended, reconciled, etc. so that a single model would be shown. Staging capabilities for different stages of a treatment plan may be shown. Various annotation tools may be available to interact with the depiction of the oral cavity.

In further embodiments, oral state capture system(s) 210 may correspond to one or more oral state capture systems 194 of FIG. 1.

In further embodiments, dental CAD system 222 may correspond to dental CAD system 154 of FIG. 1.

In further embodiments, DPMS 235 may correspond to DPMS 154 of FIG. 1.

The appliance fabrication system 225 may include one or more systems that allow appliance design and/or fabrication in embodiments. Appliance fabrication system 225 may include systems for manufacturing dental appliances and/or orthodontic appliances for patients, for example. Such dental and/or orthodontic appliances may include orthodontic aligners (e.g., clear polymeric aligners), palatal expanders, sleep apnea devices, retainers, mouth guards, night guards, and so on. In some embodiments, treatment planning and/or management system 220 generates digital models for one or more molds and/or dental appliances. The digital models for dental appliances may be used to directly print dental appliances using additive manufacturing such as 3D printings. Digital models for molds may be used to print molds associated with dental appliances. Polymeric sheets may then be thermoformed over the printed molds and trimmed to form the dental appliances in embodiments. Appliance fabrication system 225 may include 3D printers, thermoforming machines, automation machines for part movement and handling, quality control stations, and so on.

Some or all of the indicated systems may be connected via a network 250, which may include one or more public networks (e.g., the Internet) and/or private networks (e.g., intranets). Systems may exchange information such are reports, 3D model files, standard tessellation language interface (STI) files for use in 3D printing, and so on.

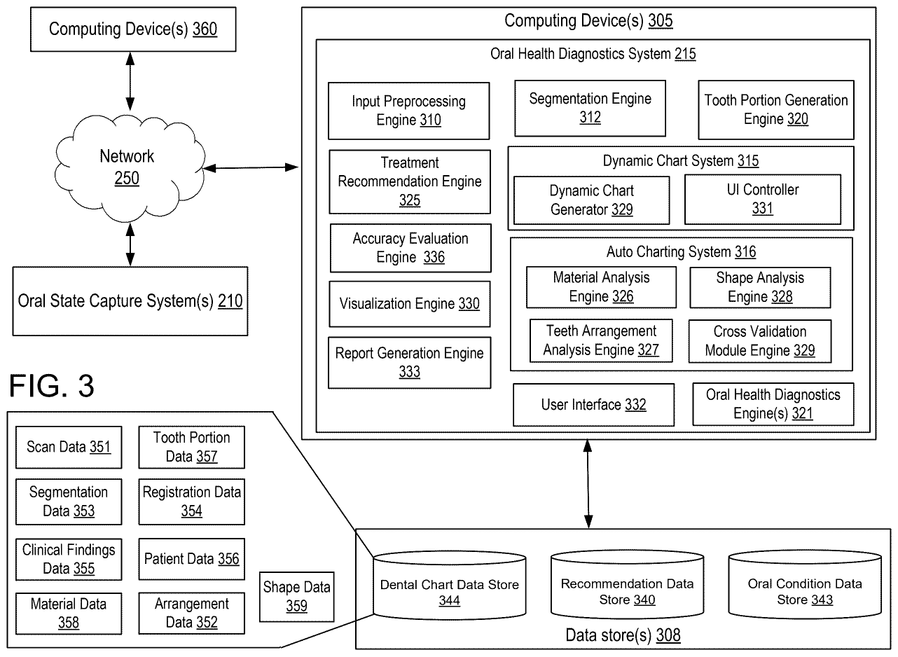

FIG. 3 illustrates an oral health diagnostics system 215, in accordance with some embodiments of the present disclosure. Oral health diagnostics system 215 may execute on one or more computing devices 305 that may be coupled to one or more computing devices 360, one or more oral state capture system(s) 210, and/or a data store 308 directly and/or indirectly via network 250 The network 350 may be a local area network (LAN), a public wide area network (WAN) (e.g., the Internet), a private WAN (e.g., an intranet), or a combination thereof. In some embodiments, oral state capture system(s) 310 connect to computing device(s) 305 directly via a wired or wireless connection.

Computing devices 305 and/or 360 may each include a processing device, memory, secondary storage, one or more input devices (e.g., such as a keyboard, mouse, tablet, and so on), one or more output devices (e.g., a display, a printer, etc.), and/or other hardware components. Computing device 305 and/or computing device(s) 360 may be connected to a data store 308 either directly or via a network (e.g., network 250). Computing device(s) 305 and/or computing device(s) 360 may include one or more physical machines and/or one or more virtual machines hosted by one or more physical machines. The physical machine(s) may be a rackmount server, a desktop computer, or other computing device. In one embodiment, computing device(s) 305 and/or computing device(s) 360 can include a virtual machine managed and provided by a cloud provider system. Each virtual machine offered by a cloud service provider may be hosted on a physical machine configured as part of a cloud. Such physical machines are often located in a data center. The cloud provider system and cloud may be provided as an infrastructure as a service (IaaS) layer. One example of such a cloud is Amazon's® Elastic Compute Cloud (EC2®). In one embodiment, oral health diagnostics system 215 is provided as software as a service (SaaS), which dental practices may subscribe to. In one embodiment, oral health diagnostics system 215 is an application that runs on a computing device (e.g., computing device 305) of a dental practice.

Data store 308 may be an internal data store, or an external data store that is connected to computing device 305 directly or via a network. Examples of network data stores include a storage area network (SAN), a network attached storage (NAS), and a storage service provided by a cloud computing service provider. Data store 308 may include a file system, a database, or other data storage arrangement.

In some embodiments, data store 308 can include a dental chart data store 344. In some embodiments, the TMD diagnostics data store 344 can include scan data 351 (e.g., CBCT scan data, intraoral scan data), segmentation data 353, registration data 354, clinical findings data 355, patient data 356, tooth portion data 357, material data 358, arrangement data 352, and/or shape data 359. In some embodiments, scan data 351, segmentation data 353, registration data 354, clinical findings data 355, patient data 356, tooth portion data 357, material data 358, arrangement data 352, and/or shape data 359 can reference a patient identifier.

In some embodiments, patient data 356 can include information identifying individual patients, including for example a patient identification number by which other data is referenced. In some embodiments, patient data 356 can store one or more generated dynamic charts, e.g., as generated by dynamic chart generator 325.

In some embodiments, scan data 351 can include image data corresponding to one or more scans (e.g., intraoral scan(s), CBCT scan(s), etc.), corresponding to one or more x-rays (e.g., panoramic x-rays), corresponding to one or more photographs (e.g., generated by a camera and/or a smart phone), corresponding to one or more sensors (e.g., generated by a compliance indicator), and/or other types of image data of a patient's dental arch(es), mouth, and/or face. In some embodiments, scan data 351 can include data generated by the oral state capture system 210. In some embodiments, scan data 351 can be used to generate a virtual model (e.g., a virtual 2D model or a virtual 3D model) of a patient's dental arch(es). Each virtual model can reflect the condition of the dental arch at a particular point in time. Each virtual model can include a 3D surface and appearance properties mapped to each point of the 3D surface (e.g., mapped to points on the 3D surface as textures).

In some embodiments, segmentation data 353 can include the segmented image data, e.g., as generated by input preprocessing engine 312, a segmented 3D model, and/or segmented intraoral scans. Segmentation may be performed using one or more trained AI models (e.g., such as neural networks), which may perform instance segmentation and/or semantic segmentation in embodiments. In some embodiments, registration data 354 can include registration data generated by input preprocessing engine 310. In some embodiments, registration data 354 can store one or more transformation matrix that indicates the rotations, translations, and/or deformations that will cause one image/3D model (e.g., generated from a first scanning session) to corresponding to another image/3D model (e.g., generated from a second scanning session).

In some embodiments, clinical findings data 355 can include information indicating clinical findings for each tooth portion, e.g., as determined by a trained AI model as further described herein. In some embodiments, tooth portion data 357 can include can store data that correspond to tooth portions, e.g., as generated by tooth portion generation engine 320.

In some embodiments, material data 358 can store data artifacts, e.g., generated by the material analysis engine 326, that characterize the material composition and/or restorative status of each tooth or tooth portion of a patient. In some embodiments, the material data 358 can include segmentation masks that delineate regions of natural tooth structure, restorative materials, and/or dental accessories, e.g., as determined through the analysis of geometrically mapped 2D color and/or NIRI images. For each tooth or tooth portion, the shape analysis engine 326 can generate and store classification labels indicating the presence and/or type of restorations (e.g., composite fillings, metal or ceramic crowns, bridges, veneers, inlays, onlays, overlays, etc.) as well as the spatial context and/or boundaries of these materials within the tooth anatomy. In some embodiments, the shape analysis engine 326 can also generate and store, as part of material data 358, quantitative metrics, such as surface area and/or volumetric estimates of restorative regions. In some embodiments, the shape analysis engine 326 can generate and store, as part of material data 358, confidence scores and/or anomaly flags associated with each classification.

In some embodiments, arrangement data 352 can store data artifacts, e.g., generated by the teeth arrangement analysis engine 327, that quantitatively describe the positional relationships and/or orientations of each segmented tooth or tooth portion within the dental arch. In some embodiments, the arrangement data 352 can include vector measurements of relative translation, rotation, and/or orientation for each tooth or tooth portion with respect to the jawline reference curve. In some embodiments, the arrangement data 352 can include inter-tooth spacing metrics, e.g., derives from Euclidean distances between adjacent tooth centroids or contact points. In some embodiments, the teeth arrangement analysis engine 327 can determine angular parameters, such as inclination and/or rotation angles, by comparing each tooth's principal axis to the tangent or normal vector of the jawline curve at the corresponding anatomical location. The angular parameters can be supplemented with alignment labels and/or anomaly flags that identify clinically relevant conditions, such as missing teeth, crowding, diastema, and/or irregular positioning. In some embodiments, the arrangement data 352 can include quantitative arrangement metrics, such as continuity, local curvature, and/or spatial outlier detection. In some embodiments, arrangement data 352 can store the jawline reference curve data, including, e.g., data that defines that geometric and/or anatomical characteristics of the dental arch baseline. This can include a mathematical representation of the curve itself, such as the coefficients or control points of a piecewise polynomial, spline, or other parametric model used to fit the spatial coordinates of the segmented teeth. This can also include the 3D coordinates of key anatomical landmarks and the sequence of points sampled along the curve, which can serve as reference positions for projecting individual teeth. Associated metadata, such sa the method of curve fitting, smoothing parameters, and/or any outlier exclusion criteria applied during generation, can also be stored. The stored data may also include tangent and/or normal vectors at each sampled point along the curve to enable the computation of relative translation, rotation, and/or orientation of teeth with respect to the jawline.

In some embodiments, shape data 359 can data artifacts, e.g., generated by the teeth shape analysis engine 328, that characterize the geometric properties and/or structural identity of one or more segmented teeth and/or dental accessories within the 3D model of a patient's dentition. In some embodiments, the shape data 359 can include shape descriptions, such as curvature maps, surface area measurements, volumetric estimates, and/or principal axis vectors, e.g., derived from the processed 3D point cloud or mesh data. In some embodiments, the shape analysis engine 328 can, for each tooth or tooth portion, generate classification labels indicating tooth type (e.g., incisor, canine, premolar, molar), primary versus permanent status, and/or the presence of non-natural or anomalous shapes associated with restorations, abutments, scan bodies, preps, and/or partially erupted teeth, for example. These classification labels can be stored in shape data 359. In some embodiments, the shape analysis engine 328 can generate segmentation masks and/or geometric feature embeddings for dental accessories, including brackets, attachments, retainers, and/or wires, based on their spatial patterns, for example. The segmentation masks and/or geometric feature embeddings can be stored in shape data 359. In some embodiments, shape data 359 can include quantitative anomaly flags and/or confidence scores for each classification and/or segmentation output.

In some embodiments, oral state capture system(s) 210 can include an intraoral scanner, a CBCT scanner (and/or another imaging device, such as a CT scanner), an electronic compliance indicator (ECI) device, a camera, a video camera, and/or optionally a computing device. In some embodiments, the computing device can be part of a scanner in the oral state capture system 210. In some embodiments, the computing device can be part of computing device 360, 305, and/or a separate device (not shown), and the oral state capture system 210 can send captured data (e.g., scan data, image data, video data) for processing on a separate device. in some embodiments, the oral state capture system 310 can include a patient or client device that can take 2D or 3D images and/or videos of the patient's anatomy in a non-clinical setting (e.g., at a patient's home). The oral state capture system 210 can obtain scan data (e.g., stored as scan data 351).

A CBCT machine is a type of x-ray machine that uses a cone-shaped x-ray beam to capture data about the patient's anatomy. The CBCT scan can generate multiple (e.g., 150-200) images form a variety of angles. In some embodiments, the data captured can be used to reconstruct a 3D image of the patient's teeth, mouth, jaw, neck, ear, nose and/or throat.

In some embodiments, oral state capture system 210 includes an intraoral scanning system comprising a scanner for obtaining intraoral scans (e.g., 3D data) of a patient's dentition and optionally a computing device. Alternatively, oral state capture system 210 may include an intraoral scanner, and the computing device may connect to the intraoral scanner to effectuate intraoral scanning. In embodiments, the computing device or another computing device of oral state capture system 210 includes an intraoral scan application that processes intraoral scans generated by the intraoral scanner to generate 3D models of the patient's upper and/or lower dental arches.

In some embodiments, the intraoral scanner may include a probe (e.g., a hand held probe) for optically capturing three-dimensional structures. The intraoral scanner may be used to perform an intraoral scan of a patient's oral cavity. An intraoral scan application running on a computing device may communicate with the scanner to effectuate the intraoral scan. A result of the intraoral scan may be scan data 351 that may include one or more sets of intraoral scans, which may include intraoral images. Each intraoral scan may include a two-dimensional (2D) or 3D image that may include depth information (e.g., a height map) of a portion of a dental site. In embodiments, intraoral scans include x, y and z information. In one embodiment, the intraoral scanner generates numerous discrete (i.e., individual) intraoral scans.

In some embodiments, the oral state capture system 210 can include an ECI device. In some embodiments, the ECI device can be used to accurately monitor of a patient's compliance to a prescribed aligner schedule. For instance, an aligner that is ECI-capable can have one or more sensors designed to detect temperature and/or proximity to a patient's tooth. The sensors can pair to a mobile phone, e.g., via a Bluetooth®-enabled “smart” aligner case, and can receive and/or transmit data between the mobile phone and the ECI. In some embodiments, the ECI device can include a pressure sensor that can measure pressure and can convert the measured physical pressure exerted on it into an electrical signal. The pressure sensor on the occlusal surface of the teeth can detect the occlusal force or biting pressure, which can be used to detect bruxism (grinding and/or clenching of the teeth). The pressure sensor can include a sensing element that directly responds to pressure, a transducer that converts the physical change in the sensing element into an electrical signal, a signal conditioning component that can amplify, filter, and/or convert the signal into a digital signal, and/or an output component that can transmit the conditioned signal to a processing device. For example, the pressure sensor can be used to measure and analyze the forces exerted during various dental procedures and treatments, such as occlusal analysis, implantology, orthodontics, prosthodontics, and/or periodontology. In some embodiments, the pressure sensor can measure electrical activity recorded during execution of a sequence of actions (e.g., bruxism-related events such as teeth clenching and teeth grinding, etc., and/or bruxism-unrelated events such as swallowing, lightly nodding the head, lightly shaking the head, speaking, etc.). In some embodiments, the pressure sensor can record a time-averaged value during execution of a particular sequence of actions. The pressure sensor can detect, record, and/or transmit signals to the computing device. The pressure data (e.g., the detected signals) can indicate clenching or grinding of a patient. In some embodiments, the pressure sensor can be attached to a processing device in oral state capture system 210, or can be otherwise connected to a processing device in oral state capture system 210.

In some embodiments, oral state capture system 210 is connected to data store(s) 308 either directly or via network 250. In some embodiments, oral state captures system 210 transmits image data (e.g., CBCT scan data, intraoral scan data, images) and/or video recording data to data store 308 for storage therein.

According to an example, a user (e.g., a dental practitioner) may subject a patient to intraoral scanning. In doing so, the user may apply an intraoral scanner to one or more patient intraoral locations. The scanning may be divided into one or more segments (also referred to as roles). As an example, the segments may include a lower dental arch of the patient, an upper dental arch of the patient, one or more preparation teeth of the patient (e.g., teeth of the patient to which a dental device such as a crown or other dental prosthetic will be applied), one or more teeth which are contacts of preparation teeth (e.g., teeth not themselves subject to a dental device but which are located next to one or more such teeth or which interface with one or more such teeth upon mouth closure), and/or patient bite (e.g., scanning performed with closure of the patient's mouth with the scan being directed towards an interface area of the patient's upper and lower teeth). Via such scanner application, the intraoral scanner may provide scan data 351 to computing device 305 (or to another computing device of oral state capture system 210). The scan data 351 may be provided in the form of intraoral scan data sets, each of which may include 2D intraoral images (e.g., color 2D images) and/or 3D intraoral scans of particular teeth and/or regions of an intraoral site. In one embodiment, separate intraoral scan data sets are created for the maxillary arch, for the mandibular arch, for a patient bite, and/or for each preparation tooth. Alternatively, a single large intraoral scan data set is generated (e.g., for a mandibular and/or maxillary arch). Intraoral scans may be provided from the intraoral scanner to the computing device 305 (or other computing device) in the form of one or more points (e.g., one or more pixels and/or groups of pixels). For instance, the intraoral scanner may provide an intraoral scan as one or more 3D point clouds. The intraoral scans may each comprise height information.

The manner in which the oral cavity of a patient is to be scanned may depend on the procedure to be applied thereto. For example, if an upper or lower denture is to be created, then a full scan of the mandibular or maxillary edentulous arches may be performed. In contrast, if a bridge is to be created, then just a portion of a total arch may be scanned which includes an edentulous region, the neighboring preparation teeth (e.g., abutment teeth) and the opposing arch and dentition. Alternatively, full scans of upper and/or lower dental arches may be performed if a bridge is to be created.

By way of non-limiting example, dental procedures may be broadly divided into prosthodontic (restorative) and orthodontic procedures, and then further subdivided into specific forms of these procedures. Additionally, dental procedures may include identification and treatment of gum disease, sleep apnea, and intraoral conditions such as malocclusions, temporomandibular joint disorder (TMD), gingival recession, tooth grinding, and so on. The term prosthodontic procedure refers, inter alia, to any procedure involving the oral cavity and directed to the design, manufacture or installation of a dental prosthesis at a dental site within the oral cavity (intraoral site), or a real or virtual model thereof, or directed to the design and preparation of the intraoral site to receive such a prosthesis. A prosthesis may include any restoration such as crowns, veneers, inlays, onlays, implants and bridges, for example, and any other artificial partial or complete denture. The term orthodontic procedure refers, inter alia, to any procedure involving the oral cavity and directed to the design, manufacture or installation of orthodontic elements at an intraoral site within the oral cavity, or a real or virtual model thereof, or directed to the design and preparation of the intraoral site to receive such orthodontic elements. These elements may be appliances including but not limited to brackets and wires, retainers, clear aligners, or functional appliances.

In embodiments, intraoral scanning may be performed on a patient's oral cavity during a visitation of a dental office. The intraoral scanning may be performed, for example, as part of a semi-annual or annual dental health checkup. The intraoral scanning may also be performed before, during and/or after one or more dental treatments, such as orthodontic treatment and/or prosthodontic treatment. The intraoral scanning may be a full or partial scan of the upper and/or lower dental arches, and may be performed in order to gather information for performing dental diagnostics, to generate a treatment plan, to determine progress of a treatment plan, and/or for other purposes. The scan data 351 generated from the intraoral scanning may include 3D scan data, 2D color images, NIR (near infrared) and/or infrared images, and/or ultraviolet images, of all or a portion of the upper jaw and/or lower jaw. The scan data 351 may further include one or more intraoral scans showing a relationship of the upper dental arch to the lower dental arch. These intraoral scans may be usable to determine a patient bite and/or to determine occlusal contact information for the patient. The patient bite may include determined relationships between teeth in the upper dental arch and teeth in the lower dental arch.

Intraoral scanners may work by moving the intraoral scanner inside a patient's mouth to capture all viewpoints of one or more tooth. During scanning, the intraoral scanner is calculating distances to solid surfaces in some embodiments. Each intraoral scan is overlapped algorithmically, or ‘stitched’, with the previous set of scans to generate a growing 3D surface. As such, each scan is associated with a rotation in space, or a projection, to how it fits into the 3D surface.

During intraoral scanning, an intraoral scan application (e.g., executing on computing device 305 or a computing device of oral state capture system 210) may register and stitch together two or more intraoral scans generated thus far from the intraoral scan sessions. In one embodiment, performing registration includes capturing 3D data of various points of a surface in multiple scans, and registering the scans by computing transformations between the scans. One or more 3D surfaces may be generated based on the registered and stitched together intraoral scans during the intraoral scanning. The one or more 3D surfaces may be output to a display so that a doctor or technician can view their scan progress thus far. As each new intraoral scan is captured and registered to previous intraoral scans and/or a 3D surface, the one or more 3D surfaces may be updated, and the updated 3D surface(s) may be output to the display. In embodiments, separate 3D surfaces are generated for the upper jaw and the lower jaw. This process may be performed in real time or near-real time to provide an updated view of the captured 3D surfaces during the intraoral scanning process.

When a scan session or a portion of a scan session associated with a particular scanning role (e.g., upper jaw role, lower jaw role, bite role, etc.) is complete (e.g., all scans for an intraoral site or dental site have been captured), the intraoral scan application may automatically generate a virtual 3D model of one or more scanned dental sites (e.g., of an upper jaw and a lower jaw). The final 3D model(s) may each be a set of 3D points and their connections with each other (i.e., a mesh). To generate a virtual 3D model, the intraoral scan application may register and stitch together the intraoral scans generated from the intraoral scan session that are associated with a particular scanning role. The registration performed at this stage may be more accurate than the registration performed during the capturing of the intraoral scans, and may take more time to complete than the registration performed during the capturing of the intraoral scans. In one embodiment, performing scan registration includes capturing 3D data of various points of a surface in multiple scans, and registering the scans by computing transformations between the scans. The 3D data may be projected into a 3D space of a 3D model to form a portion of the 3D model. The intraoral scans may be integrated into a common reference frame by applying appropriate transformations to points of each registered scan and projecting each scan into the 3D space.

In one embodiment, registration is performed for adjacent or overlapping intraoral scans (e.g., each successive frame of an intraoral video). Registration algorithms are carried out to register two adjacent or overlapping intraoral scans (e.g., two adjacent blended intraoral scans) and/or to register an intraoral scan with a 3D model, which essentially involves determination of the transformations which align one scan with the other scan and/or with the 3D model. Registration may involve identifying multiple points in each scan (e.g., point clouds) of a scan pair (or of a scan and the 3D model), surface fitting to the points, and using local searches around points to match points of the two scans (or of the scan and the 3D model). For example, the intraoral scan application may match points of one scan with the closest points interpolated on the surface of another scan, and iteratively minimize the distance between matched points. Other registration techniques may also be used. Registration data can be stored in data store 108 as a portion of scan data 351, in embodiments.

The intraoral scan application may repeat registration for all intraoral scans of a sequence of intraoral scans to obtain transformations for each intraoral scan, to register each intraoral scan with previous intraoral scan(s) and/or with a common reference frame (e.g., with the 3D model). The intraoral scan application may integrate intraoral scans into a single virtual 3D model (or two virtual 3D models, one for each dental arch) by applying the appropriate determined transformations to each of the intraoral scans. Each transformation may include rotations about one to three axes and translations within one to three planes.

The generated virtual 3D model can include color information. In some embodiments, the oral state data 351 can include color information, e.g., from 2D color images captured during the scanning process. The oral state capture system 210 can use the color information to add color texture to the 3D model(s). Once virtual 3D model(s) of the patient's dental arches are generated, they may be stored in data store 308 as a portion of scan data 351 in embodiments.

In some embodiments, computing device 305 is a desktop computer, a laptop computer, a server computer, etc., located at a doctor's office. In some embodiments, computing device 305 is a server computing device (e.g., of a data center) that may be accessed from client devices (e.g., client devices of doctors, patients, etc.). In some embodiments, computing device 305 is a virtual machine. For example, computing device 305 may be a virtual machine that runs in a cloud computing environment.

In some embodiments, oral health diagnostics system 215 includes an input preprocessing engine 310, a segmentation engine 312, an oral health diagnostics engine 321, a treatment recommendation engine 325, an accuracy evaluation engine 336, a visualization engine 330, a report generation engine 333, a user interface 332, a tooth portion generation engine 320, a dynamic chart system 315, and/or an auto charting system 316. Each of the engines and/or systems may include logic for performing one or more operations or sets of operations associated with different capabilities of the oral health diagnostics system. The division of the oral health diagnostics system 215 into various engines is provided for ease of explanation, and does not necessarily represent an actual arrangement or structure of software. For example, the operations and/or functionality described with reference to particular engines may instead be performed by other engines or modules in embodiments, and the functionality of different engines may be combined and/or divided into still further engines in embodiments. In embodiments, one or more of the engines may function as independent threads, and/or separate processes of one or more engines may function as independent threads.

Input preprocessing engine 310 can receive and process data from one or more oral state capture modalities. Processing of such data may include performing pre-processing of such data prior to providing the data to segmentation engine 312. In some embodiments, input processing engine 310 processes the input image data to determine whether it satisfies one or more image quality criteria. For example, input processing engine 310 may process the image data to determine whether a blurriness of a received image is below a blurriness threshold, whether the image data is of a proper size for processing by the segmentation engine, and so on. If the data is too small, then input processing engine 310 may add dummy pixels to the image to cause it to comply with size criteria. Similarly, if the image is too large, input processing engine may crop the image so that it satisfies size criteria. Input processing engine 310 may additionally perform one or more other alterations to input image data to place the image data into a state for improved processing by segmentation engine 312.

In some embodiments, input preprocessing engine 310 can be a software program hosted by a device (e.g., computing device 305) to process scan data 351. Input preprocessing engine 310 can perform one or more operations on scan data 351 to prepare the scan data 351 for the dynamic tooth chart and/or the automatically generated chart. Input preprocessing engine 310 can perform operations such as filtering, stabilizing, cropping, image enhancement (e.g., to sharpen an image), segmentation, and/or other operations. In some embodiments, if scan data 351 does not include a 3D model of a dental arch (e.g., includes 2D images or intraoral scans but no 3D models of dental arches), input preprocessing engine 310 can process the 2D image and/or intraoral scans to generate one or more 3D models. In some embodiments, 3D models can be generated from 2D images. In some embodiments, input preprocessing engine 310 can project 3D scan data 351 into 2D, e.g., using a mesh projection algorithm.

In some embodiments, segmentation engine 312 can be a software program hosted by a device (e.g., computing device 305) to segment the 2D scan data using 2D segmentations techniques. The resulting segmentation can then be back-projected onto the 2D model or intraoral scan, and/or stored in segmentation data 353.

In some embodiments, segmentation engine 312 can segment scan data 351 into sections of the patient's dental arch. A section can include one or more teeth of the patient, in embodiments. In some embodiments, segmentation engine 312 can receive or otherwise identify a dataset of scan data 351 that corresponds to scan data of a patient's dental arch in a particular imaging modality. Segmentation engine 312 can segment the dataset into multiple sections. In some embodiments, each section corresponds to an individual tooth, or an individual section of the dental arch corresponding to an individual tooth (e.g., if a tooth is missing, segmentation engine 312 can generate segmentation data corresponding to the area of the dental arch corresponding to the missing tooth). Segmentation data can be stored in segmentation data 353.

In some embodiments, segmentation engine 312 can include or implement a trained machine learning model that has been trained to perform semantic segmentation and/or instance segmentation of oral structures (e.g., to determine sizes, shapes, locations, tooth numbers, etc. of individual teeth, gingiva, etc.). Applicant hereby incorporates by reference the following application as if set forth fully here, as an example of a machine learning dental segmentation system and method, and training of such a machine learning segmentation system: U.S. Pat. Pub. No. 20210196434A1, published on Jul. 1, 2021, issued on Feb. 20, 2024, U.S. Pat. No. 11,903,793 B2.

In some embodiments, segmentation engine 312 can store segmentation information for scan data 351 as segmentation data 353. In some embodiments, segmentation engine 312 can be or include, for example, a trained machine learning model such as a convolutional neural network (CNN) trained to classify pixels or regions of input images into different classes. This can include performing point-level classification (e.g., pixel-level classification or voxel-level classification) of different types of features and/or objects of subjects of images. The different features and/or objects may include, for example, individual teeth, regions of the dental arch corresponding to individual teeth, gingiva, etc. The trained machine learning model of segmentation engine 312 may output one or more masks, each of which may have a same resolution as an input image. The mask or masks may include a different identifier for each identified feature or object, and may assign the identifiers on a pixel-level or patch-level basis. In one embodiment, different masks are generated for one or more different classes of features and/or objects and/or for each instance of a feature and/or object. In one embodiment, a single mask or map includes segmentation information for all identified classes of features and/or objects. Some types of features are location-specific features and are represented in one or more masks. In some embodiments, segmentation engine 312 can perform one or more processing and/or computer vision techniques or operations to extract segmentation information from images (e.g., scan data 351). Such image processing and/or computer vision techniques may or may not include the use of trained machine learning models. Accordingly, in some embodiments, segmentation engine 312 does not include a machine learning module.

Oral health diagnostics engines 321 may process outputs of the segmentation engine 312 and/or data from one or more oral state capture modalities to generate actionable symptom recommendations and/or diagnoses of oral health problems associated with identified oral conditions. In some embodiments, oral health diagnostics engines 321 include one or more trained machine learning models trained to receive inputs of identified oral conditions and/or data from one or more oral state capture modalities, and to output actionable symptom recommendations and/or diagnoses of oral health problems. In some embodiments, oral health diagnostics engines 321 include one or more decision trees and/or random forest models. Oral health diagnostics engines 312 may or may not include AI models and/or ML models. Oral health diagnostics engines 321 may receive oral condition information for multiple oral conditions, and based on the combined oral condition information may determine underlying oral health problems that may be root causes of the oral conditions or otherwise associated with the oral conditions. For example, oral health diagnostics engine(s) 321 may identify oral cancer, periodontitis, gum disease, caries, an infection around a root canal of a tooth, and/or other oral health problems.

Different oral conditions may correlate with and/or cause different oral health problems. Accordingly, information on the types of oral conditions, severity of oral conditions, etc. may be used determine actionable symptom recommendations and/or to diagnose oral health problems.

Additionally, different oral conditions and/or oral health problems may correlate with different overall health problems. Accordingly, if certain oral conditions and/or oral health problems are detected, then this may be evidence that a patient might be suffering from one or more general health conditions. For example, periodontitis has been shown to correlate with diabetes, cardiovascular disease, dementia, psoriasis, lung cancer and chronic obstructive pulmonary disease (COPD). In another example, tooth loss has been found to correlate with cardiovascular disease, COPD, and dementia. Additionally, caries has been found to correlate with diabetes and cardiovascular disease. Accordingly, detections of periodontitis, tooth loss and caries may be indicative of diabetes and/or cardiovascular disease. Similarly, detections of periodontitis and tooth loss may be indicative of COPD and dementia. If periodontitis, tooth loss and/or caries of a threshold level of severity are determined for a patient, then processing logic may output a recommendation that the patient be referred for testing to assess whether the patient has one or more of diabetes, cardiovascular disease, COPD, dementia, psoriasis and/or lung cancer in embodiments.

In an example, intraoral scans of a patient's oral cavity may be received from an intraoral scanner and one or more radiographs of the patient's oral cavity may be received from an x-ray system. One or more oral structure determination engines 315, oral condition detection engines 320 and/or oral condition mediation engines 322 configured to operate on intraoral scan data and/or 3D models generated from intraoral scan data may process the intraoral scans and/or a 3D model generated from the intraoral scans to identify gum recession, gum swelling, bleeding, caries, and/or plaque (also known as calculus) on the patient's teeth. Additionally, one or more oral structure determination engines 315, oral condition detection engines 320 and/or oral condition mediation engines 322 configured to operate on radiographs may process the radiograph(s) to identify information related to periodontal bone loss, caries, and/or calculus with respect to one or more of the patient's teeth. Oral condition detection engine 320 and/or oral condition mediation engine 322 may, for example, combine tooth segmentation information and periodontal bone loss segmentation information from analysis of a radiograph to determine the level of a periodontal bone line at one or more teeth. Oral condition detection engine 320 and/or oral condition mediation engine 322 may determine bone loss values for each tooth and/or region. Based on these bone loss values, oral condition detection engine 320 and/or oral condition mediation engine 322 may determine whether the patient has horizontal bone loss and/or vertical bone loss at one or more teeth or areas. Additionally, or alternatively, oral condition detection engine 320 and/or oral condition mediation engine 322 may determine whether the patient has generalized bone loss (e.g., that applies to all teeth in the upper and/or lower jaw) and/or whether the patient has localized bone loss (e.g., at one or more specific teeth, a particular area of the patient's jaw, etc.). Oral condition detection engine 320 and/or oral condition mediation engine 322 may additionally or alternatively determine an angle of a periodontal bone line for the patient at the one or more teeth, wherein the angle of the periodontal bone line may be used to identify at least one of horizontal bone loss or vertical bone loss.

Based on the determined periodontal bone loss information (e.g., severity of periodontal bone loss for one or more teeth, information on general vs. localized bone loss, angle of bone line, horizontal vs. vertical bone loss, etc.) from analysis of the radiograph and gum recession, gum swelling and/or plaque information from the analysis of the intraoral scan data, and optionally further based on other information such as patient age, patient habits, patient health conditions, etc., oral health diagnostics engine 321 may determine whether a patient has periodontitis and/or a stage of the periodontitis. Other received information used to assess periodontitis may include, for example, pocket depth information received from a DPMS for one or more teeth, smoking status for the patient, and/or medical history for the patient.

In embodiments, oral health diagnostics engines 321 may use information of multiple different types of identified oral conditions and/or associated severity levels to determine correlations and/or cause and effect relationships between two or more of the identified oral conditions. Multiple oral conditions may be caused by the same underlying root cause. Additionally, some oral conditions may serve as an underlying root cause for other oral conditions. Treatment of the underlying root cause oral conditions may mitigate or halt further development of other oral conditions. For example, malocclusion (e.g., tooth crowding and/or tooth spacing or gaps), tooth wear and caries may all be identified for the same tooth or set of teeth. Oral health diagnostics engine(s) 321 may analyze these identified oral conditions that have a common, overlapping or adjacent area of interest, and determine a correlation or causal link between one or more of the oral conditions. In example, oral health diagnostics engine(s) 321 may determine that the caries and tooth wear for a particular group of teeth is caused by tooth crowding for that group of teeth. By performing orthodontic treatment for that group of teeth, the malocclusion may be corrected, which may prevent or reduce further caries progression and/or tooth wear for that group of teeth. In another example, plaque, tooth staining, and gum recession may be identified for a region of a dental arch. The tooth staining and gum recession may be symptoms of excessive plaque. The oral health diagnostics engine(s) 321 may determine that the plaque is an underlying cause for the tooth staining and/or gum recession.

In embodiments, currently identified oral conditions and/or oral health problems may be used by the oral health diagnostics engine(s) 321 to predict future oral conditions that are not presently indicated. For example, a heavy occlusal contact may be assessed to predict tooth wear and/or a tooth crack in an area associated with the heavy occlusal contact. Such analysis may be performed by inputting intraoral data (e.g., current intraoral data and/or past intraoral data) and/or the oral conditions identified from the intraoral data into a trained machine learning model that has been trained to predict future oral conditions based on current oral conditions and/or current dentition (e.g., current 3D surfaces of dental arches). The machine learning model may be any of the types of machine learning models discussed elsewhere herein. The machine learning model may output a probability map indicating predicted locations of oral conditions and/or types of oral conditions. Alternatively, the machine learning model may output a prediction of one or more future oral conditions without identifying where those oral conditions are predicted to be located.

Oral health condition assessment tools may enable doctors to view and perform assessments of various types of oral conditions. Each type of oral condition may be associated with its own unique oral condition assessment tool or set of oral condition assessment tools in embodiments.

Accuracy evaluation engine 336 may determine an accuracy of one or more identified oral conditions, actionable symptom recommendations, diagnoses of oral health problems, and so on in embodiments. Alternatively, or additionally, each of the outputs of oral structure determination engines 315, oral condition detection engines 320, oral condition mediation engines 322, and/or oral health diagnostics engines 321 may include probability and/or confidence information indicating a confidence that detected oral conditions, oral health problems, etc. are correct. Confidence information may be generated for segmented objects and/or for individual pixels in embodiments.

In some embodiments, accuracy evaluation engine 336 applies one or more confidence thresholds to detections (e.g., outputs of oral condition detection engines 320). Based on the thresholds, accuracy evaluation engine 336 may determine whether objects amount to detections of oral condition classifications, and/or determine sizes of oral condition classifications in embodiments. Confidence thresholds may be automatically or manually adjusted to change the sensitivity of the system to detecting one or more types of oral conditions. In one embodiment, the oral health diagnostics system 215 may be toggled between a standard sensitivity mode and a high sensitivity mode. One or more confidence thresholds may be reduced for the high sensitivity mode as compared to the standard sensitivity mode, increasing instances of detected oral conditions and/or sizes of detected oral conditions.

In some embodiments, accuracy evaluation engine 336 may flag one or more detected oral conditions and/or diagnoses of oral health problems as suspected oral conditions and/or suspected diagnoses. Suspected oral conditions and/or diagnoses may correspond to edge cases that may be actual oral conditions/diagnoses, or may be false positives. In some embodiments, multiple different suspected diagnoses or oral condition options for a same oral condition/oral health problem may be presented, and a user may select from the multiple options. Suspected diagnoses/oral condition options may be presented as bounding boxes in embodiments. In some embodiments, suspected oral conditions/diagnoses may be presented to a user for acceptance or denial. In some embodiments, a bounding box around suspected oral conditions are transformed into a segmentation mask for the oral condition.

Treatment recommendation engine 325 may provide treatment recommendations based on detected oral conditions and/or based on determined oral health problems and/or actionable symptom recommendations. Treatment recommendation engine 325 may or may not include one or more trained machine learning models. In one embodiment, treatment recommendation engine 325 includes a decision tree that receives an input of oral condition information, diagnosed oral health problems and/or actionable symptom recommendations, and that outputs one or more treatment recommendations. Treatment recommendations may be based on combinations of different oral conditions and/or oral health problems, severity of oral conditions and/or oral health problems, patient age, patient health, and/or other parameters in embodiments.

Visualization engine 330 may output received image data of one or more oral state capture modalities to a display of a user interface 322. Visualization engine 330 may additionally generate overlays associated with each detected instance of an oral condition and output the overlays over the image data in the display. In embodiments, each instance of an oral condition may be associated with a separate layer of the overlay, and may be turned on or off individually. Visualization engine 330 may additionally output a view of a dental chart populated with oral condition information as generated by segmentation engine 312. In embodiments, visualizations may include overlays having shapes of areas of interest for identified oral conditions. These areas of interest may be displayed using a visualization that is coded based on classes of the one or more oral conditions that the areas of interest are associated with.

In some embodiments, visualization engine 330 generates bone measure visualizations, which may show an amount of periodontal bone loss of a patient. Oral condition detection engines 320, oral condition mediation engines 322 and/or oral health diagnostics engines 321 may operate on data to perform bone measure analysis in embodiments. The result of such analysis may then be shown by the visualization engine 330. Such bone measure visualizations may show how a patients'teeth retain or lose bone density over time using analysis of data from radiographs and/or other oral state capture modalities. In embodiments, bone loss may be measured and shown on panoramic x-rays or periapical x-rays, and/or from current bite-wing x-rays combined with data from older panoramic or periapical x-rays or older intraoral scans.

In some embodiments, tooth portion generation engine 320 can be a software program hosted by a device (e.g., computing device 305) to generate units of data that can be combined to create the dynamic tooth chart. In some embodiments, tooth portion generation engine 320 can identify datasets of scan data 351 that include scan data of a dental arch of a patient. The scan data in each dataset can correspond to an imaging modality (e.g., intraoral scan, CBCT, photograph, video, radiograph, etc.). The scan data in each dataset can provide information about a patient's dentition. In some embodiments, the tooth portion generation engine 320 can identify the segmentation data 353 corresponding to the dataset. The segmentation data 353 can identify segments (or portions) of the patient's dental arch. For example, a segment can include one tooth or multiple teeth of the patient's dental arch.

In some embodiments, tooth portion generation engine 320 can normalize each section, e.g., for size, scale, color balance, brightness, and/or orientation, for coordinated presentation on the dental chart. That is, each section can be normalized to fit within the dental chart format. Tooth portion generation engine 320 can then generate a tooth portion unit for each normalized section, and can store the tooth portion units in tooth portion data 357. Each tooth portion unit of data can correspond to a section of the dental chart in a particular imaging modality. The tooth portion generation engine 320 can generate a tooth portion unit for each modality. In some embodiments, each tooth portion unit of tooth portion data 357 can include an image of one or more teeth of the patient, representing a particular imaging modality. Each tooth portion unit can automatically align with other tooth portion units within the dental chart format, e.g., in a snap-to-fit configuration. For example, each tooth portion unit representing a specific section of the dental chart in a particular modality can be automatically aligned and positioned within the overall chart layout (e.g., without manual adjustment by the user). After normalizing for parameters such as size, scale, color balance, brightness, and/or orientation, each tooth portion unit is configured to seamlessly “snap” into its designated location on the dental chart. The snap-to-fit configuration can enable images from different modalities, or from different time points, to be consistently and accurately displayed in relation to one another, preserving anatomical relationship and visual coherence across the dental chart. In some embodiments, the tooth portion generation engine 320 can assign spatial coordinates or grid positions to each tooth portion unit, e.g., based on anatomical landmarks or predefined chart templates. These spatial coordinates or grid positions can enable each tooth portion unit to be automatically positioned within the dental chart, in such a way that adjacent units abut seamlessly, preserving anatomical relationship and reducing (or eliminating) gaps or overlaps. This snap-to-fit configuration can allow for the dynamic assembly of the chart from heterogeneous data sources, enabling images from different modalities or time points to be displayed in a coherent, standardized manner.

In some embodiments, tooth portion generation can be done using 3D registration and/or image registration. In some embodiments, 3D registration can include capturing 3D data of various points in multiple images (e.g., in multiple scans of the same patient), and registering the images by computing transformations between the images/scans. Registering each tooth can offset any tooth movement that may have occurred between scans of the patient's dentition. In some embodiments, image registration involves identifying multiple points, point clouds, edges, corners, surface vectors, etc., in each image/scan of an image/scan pair, surface fitting to the points of each image/scan, and using local searches around points to match points of the two images/scans.

In some embodiments, the dynamic chart system 315 can include a dynamic chart generator 329 and/or a UI controller 331.

In some embodiments, dynamic chart generator 329 can be a software program hosted by a device (e.g., computing device 305) to identify tooth portion units of tooth portion data 357 for a particular patient, arrange the tooth portion units into a dental chart, and/or create a visualization of the dental chart of the patient. In some embodiments, dynamic chart generator 329 can identify a particular imaging modality and select the tooth portion units of tooth portion data 357 that correspond to the identified imaging modality. In some embodiments, dynamic chart generator 329 can identify the particular imaging modality based on the context of the patient's visit to the doctor's office. Examples of context include the chief complaint of the patient, a treatment plan, a treatment type, a scheduled dental visit (e.g., a regular 6-month check-up, a tooth cleaning), an input of a user (e.g., of the dental practitioner), or a detection of a condition (e.g., identified by AI). In some embodiments, the dynamic chart generator 329 can identify a correlation between the context of the visit and each of the imaging modalities, and can identify the particular imaging modality based on a ranking of the correlations.

As an illustrative example, a dental practitioner could input the context of the visit as identifying caries. The dynamic chart generator 329 can identify bite wing x-rays as having the highest correlation to the context for the buccal view, can identify NIR images as having the highest correlation to the context for the occlusal view, and can identify the panoramic x-ray has having the highest correlation to the context for the lingual view. That is, the bite-wing x-rays can be the best imaging modality for displaying caries in the buccal view, the NIR images can be the best imaging modality for displaying caries in the occlusal view, and the panoramic x-rays can be the best imaging modality for displaying caries in the lingual view. Thus, the dynamic chart generator 329 can identify the tooth portion units of tooth portion data 357 corresponding to the bite-wing x-rays of the buccal views of the patient, the tooth portion units of tooth portion data 357 corresponding to the NIR images of the occlusal view of the patient, and the tooth portion units of tooth portion data 357 corresponding to the panoramic x-rays of the lingual view of the patient when generating the visualization of the patient's dental chart. If any of the imaging modalities are not available for the particular patient (or are not available for the particular view for the patient), the dynamic chart generator 329 can select the imaging modality with next highest correlation. In some embodiments, the dynamic chart generator 329 can generate a visualization of the patient's dental chart using multiple imaging modalities (e.g., two or more), or can generate a visualization of the patient's dental chart using a single imaging modality.

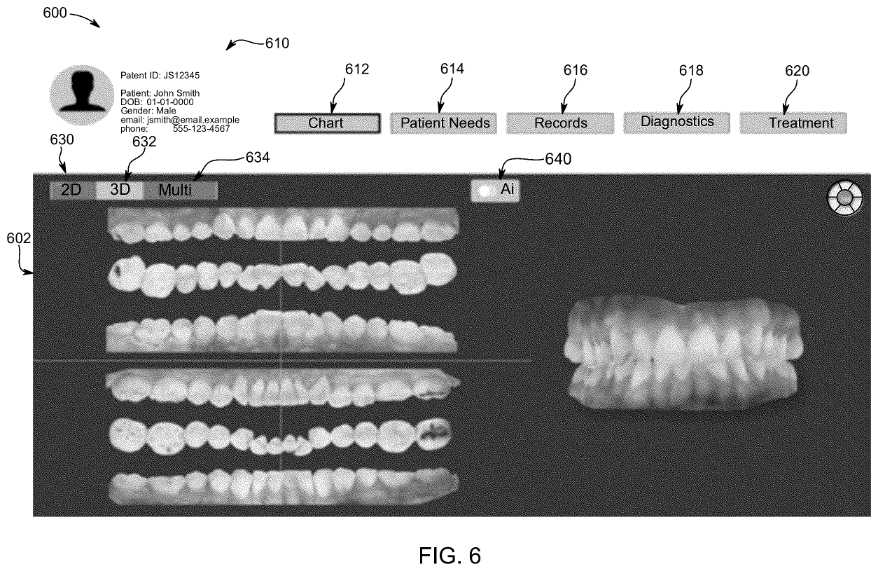

In some embodiments, the visualization of the dental chart can include a buccal view, an occlusal view, and/or a lingual view of the dental arch of the patient, and can optionally include a 2D view, a 3D view, and/or a multi-dimensional view. Examples of the visualization of the dental chart generated by dynamic chart generator 329 are described with respect to FIGS. 6-11.

In some embodiments, the dynamic chart generator 329 can modify the visualization of the patient's dental chart, e.g., based on a user interaction received from the user device (e.g., via the UI). In some embodiments, the dynamic chart generator 329 can identify a second imaging modality that corresponds to the user interaction. For example, the user can change the context of the visit, e.g., to input the patient's secondary complaint. The dynamic chart generator 329 can identify imaging modality (or modalities) that correspond to the user input, and can generate an updated visualization using the tooth portion units of tooth portion data 357 in the newly identified imaging modality (or modalities).

In some embodiments, the tooth portion data 357 can include data from multiple image sources of the same imaging modality. The dynamic chart generator 329 can identify the image source that corresponds to the imaging modality that best corresponds to the context. In some embodiments, the dynamic chart generator 329 can include an AI model that can receive, as input, the image sources corresponding to particular imaging modalities, and can identify which image of the multiple images of each imaging modality best represents the context. For example, if the context is to identify caries, the AI model can identify which image of the multiple images of bite-wing x-rays of the buccal view of the patient's dental arch best displays the caries. In some embodiments, the AI model can rank the images, e.g., from best to worst. In some embodiments, the AI model can rank the images for each tooth portion unit. Thus, as an illustrative example, the dynamic chart generator 329 can use the tooth portion unit of tooth portion data 357 from one image to display a first tooth of the patient's dental arch, and can use the tooth portion unit of tooth portion data 357 from a second image to display the a second tooth of the patient's dental arch.

In some embodiments, the dynamic chart generator 329 can process scan data 351 (or a subset thereof, e.g., corresponding to the patient) to detect oral condition(s) of the patient, such as, cavities, gum disease, tooth wear, cracked or fractured teeth, plaque or tartar build-up, bite issues, temporomandibular joint disorder, impacted teeth, orthodontic needs, etc. In some embodiments, the dynamic chart generator 329 can use an AI model that is trained to provide the detected conditions. In some embodiments, the dynamic chart generator 329 can update the tooth portion units of tooth portion data 357 to include a representation of the detected conditions in the visualization of the dental arch of the patient.

In some embodiments, the dynamic chart generator 329 can provide, as input to an AI model, the scan data 351 that corresponds to the patient. The AI model can be trained to provide an indication of a clinical finding corresponding to particular imaging modality. The dynamic chart generator 329 can receive, as output from the AI model, the indication of the clinical finding. In some embodiments, the clinical finding can be stored as clinical findings data 355 of data store 308. In some embodiments, the dynamic chart generator 329 can include the clinical finding in the visualization of the dental chart of the patient. In some embodiments, the dynamic chart generator 329 can include the clinical finding in a second imaging modality that best corresponds to the clinical finding.

In some embodiments, the AI model an provide multiple indications of a clinical finding. Each indication can correspond to a particular imaging modality. For example, the AI model can provide a first indication of a cavity in a first imaging modality (e.g., intraoral scan image), and a second indication of the cavity in a second imaging modality (e.g., NIR image). To determine which image data to include in the visualization to indicate the clinical finding, the dynamic chart generator 329 can identify the imaging modality that best corresponds to the clinical finding, and can identify the indication that corresponds to that imaging modality. The dynamic chart generator 329 can then identify the tooth portion unit of tooth portion data 357 that corresponds to that imaging modality, and include an indication of the clinical finding in that tooth portion unit.

In some embodiments, the dynamic chart generator 329 can aggregate the multiple indications of the clinical finding to generate an overall indication, and include the overall indication of the clinical finding in the visualization of the patient's dental chart.

In some embodiments, the dynamic chart generator 329 can provide image data of multiple image sources corresponding to scan data 351 to an AI model that is trained to provide a confidence score associated with an identified clinical finding (e.g., identified using an AI model, or otherwise identified) for each of the image source. The dynamic chart generator 329 can then identify an image source with the highest confidence score, and can include that tooth portion unit corresponding to that image source in that visualization of the dental chart.

In some embodiments, the tooth portion units of tooth portion data 357 can correspond to a particular point in time, and the dynamic chart generator 329 can generate a visualization of the patient's dental chart that corresponds a particular point in time. For example, the visualization can show the patient's dental chart at a past time (e.g., 6 months ago), or at the current time.

In some embodiments, user interface (UI) controller 331 can provide the visualization of the patient's dental chart (e.g., generated by dynamic chart generator 329) to a user device (e.g., computing device 160) for presentation in a user interface (e.g., UI 332). In some embodiments, UI controller 331 can enable a user to interact with the visualization, e.g., by focusing on a particular section of the dental arch, changing imaging modalities, changing the point in time, changing between a 2D, 3D or multi-dimensional view, incorporating AI findings, etc.

In some embodiments, the UI controller 331 can enable a user to make modifications to particular aspects of the patient's dentition or dental arch, and cause the dynamic chart generator 329 to generate an updated visualization that corresponds to the modifications. In some embodiments, the UI can include a measurement tool that enables a user to measure (e.g., in 3D) the size of each tooth. In some embodiments, the measurement tool can enable the user to modify the measurements or dimensions of a tooth. The modifications can then be used by the dynamic chart generator 329 to generate an updated visualization that reflects the modified measurements or dimensions, and the UI controller 331 can provide the updated visualization to the user device.

As an illustrative example, an orthodontist may measure the height of central teeth, lateral teeth, and cuspid teeth, with the goal of achieving a specific ratio between the measurements. The UI controller 331 can provide tools to display the effects of 3D movements from each angle (e.g., occlusal, lingual, and/or buccal) simultaneously.

In some embodiments, the UI controller 331 can provide multiple views of the dynamic tooth chart, e.g., 2D, 3D, multi-dimensional view, views based on modalities, based on a particular condition, based on the context for the visit, and/or a combination thereof. In some embodiments, the UI controller 130 can enable a user to create a custom view of the dynamic tooth chart. In some embodiments, the UI controller 130 can provide templates to enable a user to create custom view(s).

In some embodiments, computing device(s) 360 can be or include a user device. In some embodiments, the user device 160 can be used by a dental professional (e.g., a doctor, a dentist, a hygienist, a clinician, and/or a technician, sometimes referred to herein as a dental practitioner) to educate a patient regarding the patient's dental health. The user device 360 can include a user interface (UI) 332 to display the generated dynamic chart, and optionally include tools to interact with and/or modify the chart.

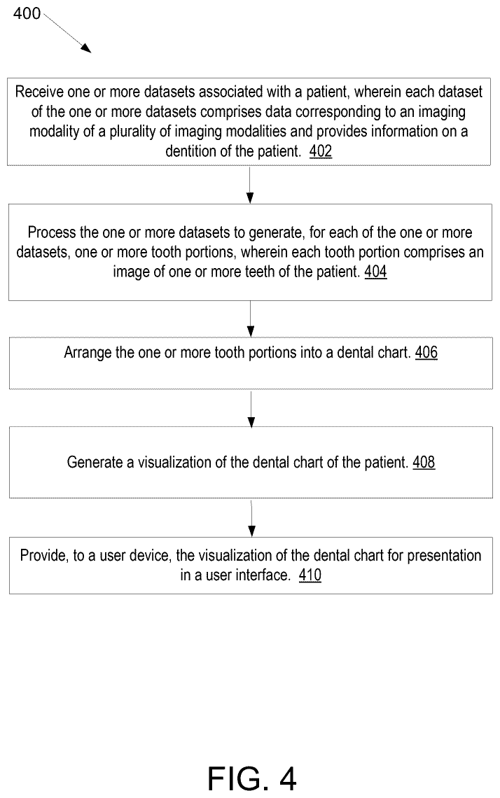

The dynamic chart system 315 is further described with respect to FIGS. 4-11.

In some embodiments, auto charting system 316 can be a software program hosted by a device (e.g., computing device 305) to automatically generate tooth annotations used to populate a dental chart. In some embodiments, auto charting system 316 include a material analysis engine 326, a teeth arrangement analysis engine 327, and/or a shape analysis engine 328. The material analysis engine 326 can perform a material analysis based on 2D color and/or NIRI images, by segmenting the teeth according to material. The result of the material analysis can apply to global treatment history for the patient, such as the presence of a crown and/or bridges, as well as local treatments such as partial restoration, veneer, inlay, onlay, overlay, and/or significant fillings. The teeth arrangement analysis engine 327 can perform a teeth arrangement analysis based on 3D model(s) of the patient's dentition by comparing the teeth position to a jaw arc model and detecting missing teeth and/or irregular tooth position. The shape analysis engine 328 can perform a shape analysis based on the 3D dental model(s) (and optionally color and/or NIRI attributes) to identify tooth identifications, including, for example, primary teeth, analysis of reconstruction entities such as an abutment, scan-body, and/or prep, as well as partially hatched teeth and detection of dental accessories such as brackets, attachments, retainer, and/or braces.

In some embodiments, material analysis engine 326 can assess dental structures by analyzing the composition and/or material characteristics of each tooth. The material analysis engine 326 can receive (e.g., from segmentation engine 312), retrieve (e.g., from segmentation data 353), or otherwise identify segmentation data identifying individual teeth and their spatial context within a 3D model of the patient's dentition. The material analysis engine 326 can received, retrieve (e.g., from scan data 351), or otherwise identify 2D color images and/or NIRI images of the patient. The material analysis engine 326 can geometrically map the 2D color images and/or NIRI images to the segmented 3D model of each tooth. In some embodiments, the material analysis engine 326 can perform a 2D color analysis that includes projected the segmented 3D tooth models onto corresponding 2D color intraoral images. In some embodiments, the color analysis can include using image processing and/or ML models (e.g., CNNs or U-Net architecture) to extract and analyze colorimetric features within each tooth region. The analysis can focus on differentiating the optical properties of various dental materials, including, for example, the hue, saturation, and/or brightness characteristics that distinguish natural enamel and dentin from restorative materials such as composite resin, amalgam, ceramic, and/or porcelain. The color analysis can enable the material analysis engine 326 to localize findings to specific dental surfaces (e.g., buccal, lingual, occlusal). The 2D color analysis can result in material detection, such as tooth, restorative, tartar, etc. The material analysis engine 326 can perform a material segmentation to identify (e.g., detect and segment) veneer, inlay, onlay, overlay, fillings, etc.

In some embodiments, the material analysis engine 326 can receive input from the segmentation engine 312, which can provide detailed segmentation data identifying individual teeth and their spatial context within the 3D model of the patient's dentition. The material analysis engine 326 can process multi-modal scan data, including 2D color images and/or NIRI images, which can be geometrically mapped to the segmented 3D model using intrinsic and extrinsic calibration parameters or direct transformation matrices. The material analysis engine 326 can analyze the optical properties, such as colorimetric values, translucency, and/or reflectance patterns, captured in these images to distinguish between natural tooth material and a variety of restorative materials, including but not limited to composite fillings, metal or ceramic crowns, bridges, veneers, inlays, onlays, and/or overlays. The material analysis engine 326 can use image processing algorithms and/or ML models, such as convolutional neural networks or U-Net architectures, to segment and classify regions of each tooth according to their material composition. In some embodiments, the image processing algorithms and/or ML models can segment and classify regions of each tooth according ot their material composition even where restorations are subtle, partially blended with natural tissue, or present in complex multi-material configurations. The material analysis engine 326 can generate detailed material labels and/or segmentation masks for each tooth, e.g., indicating the presence, type, and/or spatial extend of restorations or artificial structures. In some embodiments, the material labels and/or segmentation masks can be stored in data store 308, e.g., as material data 358. These results can be integrated with outputs from the shape analysis engine 328 and/or the teeth arrangement analysis engine 327 to produce a comprehensive, multi-dimensional dental chart. The material analysis engine 326 can provide precise differentiation of materials, thereby enhancing accuracy of the automated charting process and supplying data for diagnostic algorithms and/or treatment planning modules within the oral health diagnostics system 215.

In some embodiments, teeth arrangement analysis engine 327 can perform spatial analysis of dental structures by evaluating the positional relationships of individual teeth relative to the anatomical curvature of the jaw. The material analysis engine 326 can receive (e.g., from segmentation engine 312), retrieve (e.g., from segmentation data 353), or otherwise identify segmentation data and tooth identification within a 3D model of the patient's dentition. The teeth arrangement analysis engine 327 can generate a reference jawline curve. In some embodiments, the teeth arrangement analysis engine 327 can generate the jawline reference curve by first extracting the spatial coordinates of the segmented teeth and the relevant anatomical landmarks from the 3D model of the patient's dentition. Using these data points, the teeth arrangement analysis engine 327 can apply a model, such as a piecewise polynomial, spline interpolation, or least-squares fitted curve, to approximate the natural curvature of the dental arch. The process may involved identifying the central points or centroids of each tooth, as well as the boundaries of the alveolar ridge, for anatomical accuracy. In some embodiments, the teeth arrangement analysis engine 327 can employ outlier detection and/or smoothing algorithms to minimize the influence of missing teeth, mal-positioned teeth, and/or artifacts, to produce a continuous, anatomically representative baseline.

In some embodiments, the teeth arrangement analysis engine 327 can compute the relative translation, rotation, and/or orientation of each tooth with respect to this baseline. For example, the teeth arrangement analysis engine 327 can project the centroid of each tooth onto the jawline reference cruve to determine its expected anatomical position along the dental arch. The teeth arrangement analysis engine 327 can quantify relative translation by calculating the vector displacement between the actual centroid of the tooth and its corresponding point on the reference curve, capturing deviations in mesiodistal, buccolingual, and/or occlusogingival directions. For rotation and orientation, the teeth arrangement analysis engine 327 can perform a comparative analysis between the principal axes of the tooth and the tangent or normal vectors of the jawline curve at the projected location, sing metrics such as Euler angles or rotation matrices to describe angular discrepancies. In some embodiments, the teeth arrangement analysis engine 327 can analyze inter-tooth spacing, angulation, and/or alignment to detect clinically relevant conditions, such as missing teeth, crowding, diastema, and/or irregular positioning. For example, the teeth arrangement analysis engine 327 can calculate inter-tooth spacing by measuring the Euclidean distances between the centroids or contact points of adjacent teeth along the dental arch, comparing these values to normative anatomical thresholds to identify deviations indicative of diastema (excessive spacing) or crowding (reduced spacing). For angulation analysis, the teeth arrangement analysis engine 327 can determine the orientation of each tooth's principal axis relative to the tangent or normal vector of the jawline reference curve at the corresponding position, using angular metrics such as inclination or rotation angles to detect irregular tilting or malposition. In some embodiments, the teeth arrangement analysis engine 327 can assess alignment by evaluating the sequential arrangement of teeth along the arch, identifying discontinuities or gaps that may signal missing teeth, as well as detecting outliers whose spatial or angular parameters fall outside clinically accepted ranges.

In some embodiments, the teeth arrangement analysis engine 327 can integrate contextual information from the material analysis engine 326 and/or the shape analysis engine 328, allowing for more robust differentiation between true anatomical anomalies and artifacts resulting from restorations or dental accessories. In some embodiments, the teeth arrangement analysis engine 327 can generate spatial labels, arrangement metrics, and/or anomaly flags for each tooth. In some embodiments, the teeth arrangement analysis engine 327 can store the labels, metrics, and/or flags in arrangement data 352 of data store 308. In some embodiments, the labels, metrics, and/or flags can be incorporated into the comprehensive dental chart.

In some embodiments, the teeth arrangement analysis engine 327 can be or implement a an ML model (e.g., a convolutional neural network or a point cloud-based architecture) specifically trained for dental spatial analysis. The teeth arrangement analysis engine 327 can provide, as input ot the ML model, segmented 3D data of the dental arches, e.g., including the spatial coordinates and orientation vectors of individual teeth, and optionally contextual features such as the jawline reference curve and/or inter-tooth distances. In some embodiments, the input data may be further augmented with derived features, such as local curvature, tooth centroid positions, and/or principal axes, e.g., to enhance the model's ability to capture spatial relationships and anatomical context. In some embodiments, the ML model can be trained on a training dataset that includes clinically labeled conditions such as missing teeth, crowding, diastema, and/or irregular positioning. The ML model can learn complex patterns of normal and abnormal tooth arrangement. The ML model can provide, as output, structured predictions for each tooth and inter-tooth region, e.g., including classification labels (e.g., normal, crowded, spaced, missing), quantitative metrics (e.g., deviation from expected position, angulation angles), and/or anomaly flags. In some embodiments, the output of the ML model can be directly integrated into the comprehensive dental chart.

In some embodiments, shape analysis engine 328 can perform morphological assessment of dental structures by analyzing the geometric features of each segmented tooth within the 3D model of the patient's dentition. In some embodiments, the shape analysis engine 328 can receive (e.g., from segmentation engine 312), retrieve (e.g., from segmentation data 353), or otherwise identify segmentation data and tooth identification within a 3D model of the patient's dentition. The segmentation data can include high-resolution 3D point cloud or mesh data corresponding to individual teeth and their spatial context. The shape analysis engine 328 can extract and process geometric descriptors, such as curvature, surface area, volume, and/or principal axes, from the 3D data to characterize the morphology of each tooth. In some embodiments, the shape analysis engine 328 can implement one or more ML models, such as point cloud neural networks or feature embedding architectures, trained to classify teeth according to type (e.g., incisor, canine, premolar, molar), identify primary versus permanent teeth, and/or detect non-natural and anomalous shapes indicative of dental restorations, abutments, scan bodies, preps, and/or partially erupted teeth. In some embodiments, the shape analysis engine 328 can identify and/or segment dental accessories, such as brackets, attachments, retainers, and wires, e.g., by recognizing their distinctive geometric patterns within the 3D data. The outcomes produced by the shape analysis engine 328 can include detailed shape-based classification labels, anomaly flags, and/or segmentation masks for each tooth and accessory. The shape analysis engine 328 can store the labels, flags, and/or masks in data store 308, e.g., as shape data 359. In some embodiments, the labels, flags, and/or masks can be integrated with outputs of the material analysis engine 326 and/or the teeth arrangement analysis engine 327.

In some embodiments, the cross validation module 329 can perform cross-validation based on symmetry and/or multiple scans of the same patient. In some embodiments, the cross validation module 329 can serve as a quality assurance component by systematically evaluating and reconciling the outputs generated by the shape analysis engine 328, the material analysis engine 326, and/or the teeth arrangement analysis engine 327. In some embodiments, the cross valuation module 329 can receive (e.g., from engines 326-328), retrieve (e.g., from data store 308), and/or otherwise identify detailed classification labels, segmentation masks, spatial arrangement metrics, and/or material composition data. In some embodiments, the cross validation module 329 can implement one or more validation algorithms, such as symmetry checks that compare the morphology and/or material properties of contralateral teeth, consistency checks that assess the alignment between shape-based and/or material-based classifications, and/or temporal comparisons that evaluate the stability of findings across multiple scans of the same patient. The cross validation module 328 can use geometric registration techniques, such as iterative closest point algorithms, to align and compare mirrored tooth models, and can apply statistical or machine learning-based anomaly detection methods to flag discrepancies or outlier. The cross validation module 329 can generate confidence scores, correction suggestions, and/or validation flags for each tooth and/or dental structure, which can be fed back into the system for automated correction and/or presented to dental practitioners for review.

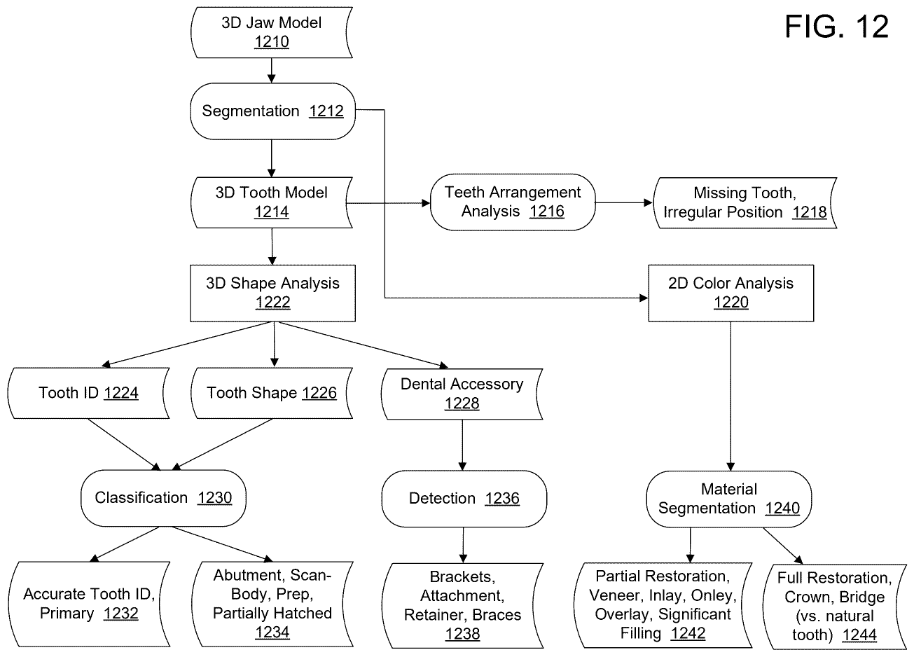

The auto charting system 316 is further described with respect to FIGS. 12-20.

In some embodiments, the results of the auto charting system 316 can be used to improve other diagnostic systems, such as reducing false positives in caries detection by recognizing restorations, informing time lapse analysis by identifying when a tooth has been restored or replaced, adjusting tooth strength assessment algorithms based on implant detection, and/or detecting missing attachments by comparing charting results to treatment plans. For example, by accurately identifying the presence, type, and spatial extent of restorations (such as fillings, crowns, bridges, veneers, inlays, and onlays), the outcome of the auto charting system 316 can enable caries detection algorithms to distinguish between natural tooth structure and restorative materials. This differentiation can help reduce false positives, as caries-like features in imaging modalities (e.g., NIRI or radiographs) may arise from the optical or geometric properties of restorations, rather than true pathological lesions. Thus, by referencing auto charting annotations, the caries detection algorithm can suppress and/or reclassify suspicious regions that overlap with known restorations, thereby improving diagnostic specificity. As another example, the identification of teeth that have been restored or replaced (e.g., by crowns, bridges, or extractions) can provide context for a time lapse analysis that tracks morphological changes in dentition over time. By correlating detected changes in tooth shape or position with auto charting records of restorative interventions, the time lapse algorithm can differentiate between clinically significant disease progression (such as wear or fracture) and expected alterations due to restorative procedures, thus reducing misinterpretation and supporting more accurate longitudinal assessments. As another example, the detection of dental implants through auto charting can allow tooth strength assessment algorithms (such as those evaluating vertical mobility or occlusal load response) to dynamically adjust their analytical thresholds and/or interpretive criteria. Since implants are expected to exhibit minimal physiological movement compared to natural teeth, the algorithm can flag any detected mobility as a potential clinical concern, while avoiding false alarms for natural teeth with normal physiological movement. As another example, by comparing auto charting results to planned or expected attachment locations from orthodontic treatment plans, the system can automatically detect missing or dislodged attachments (e.g., aligner attachments or brackets). This comparison enables timely identification of deviations from the prescribed treatment protocol, supporting clinical intervention and improving treatment outcomes. Thus, the auto charting system 316 can serve as a foundational data layer that augments the accuracy, context-awareness, and clinical utility of a broad range of dental diagnostic algorithms.

In some embodiments, the auto charting system 316 can remove and/or inpaint detected dental accessories (e.g., attachments, orthodontic brackets, archwires, bonded retainers, etc.) in the visualization of the dental chart, e.g., to facilitate a cleaner analysis. For example, upon identifying a dental accessory (e.g., using feature-based object detection algorithms and/or trained ML models), the auto charting system 316 can segment the affected region form the surrounding dental and/or gingival structures. In some embodiments, the auto charting system 316 can remove the identified accessory by excising the accessory geometry or masking the corresponding pixels/voxels to isolate the area of interest. In some embodiments, the auto charting system 316 can perform an inpainting process using, e.g., context-aware interpolation, patch-based synthesis, and/or deep learning-based image and surface completion models, to reconstruct the occluded regions by inferring the most probable underlying tooth surface or gingival tissue morphology and/or appearance. This restoration process can yield a natural, anatomically plausible representation that closely approximates the original unaltered oral anatomy. In some embodiments, the removal and/or inpainting of dental accessories can mitigate artifacts and confounding features that can adversely impact the performance of subsequent analysis modules, e.g., semantic segmentation, morphological shape analysis, and/or AI-driven diagnostic algorithms. For example, by presenting a normalized dataset free from accessory-induced distortions, the auto charting system 316 can enhance the accuracy and robustness of tooth identification, restorative status classification, and/or pathology detection.

The user interface 332 can be a graphical user interface and may include icons, buttons, graphics, menus, windows and so on for controlling and navigating the oral health diagnostics system 215. A user may interact with user interface 332 to select individual teeth, to modify instances of oral conditions (e.g., by redrawing their shape), to remove instances of oral conditions, to add instances of oral conditions, to turn on and off overlay layers, and so on. The user interface 332 may include tools that a doctor or other user can use to model, annotate, and/or otherwise interact with various oral structures and/or oral conditions that are imaged through various oral structure capture modalities, including radiographs.

User interface 332 may provide visualizations generated by visualization engine 330 about oral structures (e.g., teeth), or oral conditions, oral health problems, and so on. The visualizations may be associated with tools for manipulating oral structures and/or oral conditions, for selecting oral structures, oral conditions, actionable symptom recommendations, diagnoses, and so on. Via the user interface 332, doctors may provide input about oral structures, oral conditions, actionable recommendations, diagnoses, and so on.

User interface 332 enables users to interact with various forms of data that capture the state of a patient's dentition. User interface 332 may additionally enable users to plan treatments for a patient's dentition with various oral state capture modalities, including x-rays. User interface 332 may provide multiple visualization tools that a doctor or other user can use to model, annotate, and/or otherwise interact with various oral structures that are imaged through various oral structure capture modalities, including radiographs. User interface 332 may also provide treatment planning tools for planning of patient treatments. For example, user interface 332 may receive a selection of a treatment recommendation, and oral health diagnostics system 215 may initiate and/or perform automated treatment planning based on the selected treatment recommendation (e.g., optionally including interfacing with a treatment planning system).

In an example, one or more treatment recommendations comprise at least one of one or more restorative treatment recommendations or one or more orthodontic treatment recommendations. Oral health diagnostics system 215 may receive a selection of at least one of a restorative treatment recommendation of the one or more restorative treatment recommendations or an orthodontic treatment recommendation of the one or more orthodontic treatment recommendations based on user interaction with user interface 332. Oral health diagnostics system 215 may then generate a treatment plan that is one of a restorative treatment plan, an orthodontic treatment plan, or an ortho-restorative treatment plan based on the selection. Generating the treatment plan may include determining staging for the treatment plan, optionally receiving modifications to one or more stages of the treatment plan, and outputting an updated treatment plan in an example.

In some embodiments, user interface 332 provides one or more interactive elements to facilitate interaction with a segmented radiographic representation of the oral cavity. User interface 332 may receive one or more interactions with the segmented radiographic representation through the one or more interactive elements, and oral health diagnostics system 215 may take one or more actions, implement one or more recommendations, take one or more treatment steps, etc. based on the one or more interactions.

Via the user interface 332, a user may additionally cause a report to be generated, cause data to be exported to one or more other systems, cause data to be stored, toggle between a standard sensitivity mode and a high sensitivity mode, and so on. The user interface 332 can provide a platform for a doctor or other user to model, annotate, and/or interact with various oral structures depicted through processing of the oral state capture modalities.

User interface 332 may provide multiple different types of interactions that users can have with the depictions of oral structures and oral conditions identified in image data. Such interactions may include rotations, movements of jaws, zooming in, zooming out, panning in one or more directions, and so on. In some embodiments, user interface 332 provides staging information, treatment planning information (e.g., for ortho-restorative treatment) and/or controls for treatment planning and/or treatment management based on integration with a treatment planning system and/or treatment management system. User interface 332 may output photo-realistic depictions of treatment and/or staging based on data generated by visualization engine 330 in embodiments.

In some embodiments, user interface 332 provides information about oral conditions and/or oral structures (e.g., teeth) responsive to a user causing a pointer to hover over the oral conditions and/or oral structures in a presented image. Controls for modifying oral conditions, sizing oral conditions, etc. may additionally or alternatively be presented responsive to a user hovering a pointer over an oral condition or tooth and/or responsive to a user selecting (e.g., via clicking, double clicking, etc.) an oral condition or tooth. User interface 332 may provide controls for sharing a state of a treatment (e.g., with a patient, another doctor, etc.). User interface 332 may provide controls for making changes or modifications to treatments. In an example, user interface 332 may provide controls for moving attachments, teeth, etc. In embodiments, user interface 332 may provide controls for designing restorations or other dental appliances. In embodiments, user interface 332 may provide controls for sending fabrication instructions to fabricate dental appliances, restorations, orthodontic aligners, palatal expanders, wires and brackets, and so on.

In some embodiments, user interface 332 presents one or more radiographs of a patient's oral cavity, and provides controls for annotating the radiograph(s). In some embodiments, user interface 332 outputs one or more visual overlays over a radiograph or other image data based on visualizations generated by visualization engine 330. The visual overlays may convey information about oral conditions, severity of oral conditions, locations of oral conditions, types of oral conditions, and so on. The visual overlays may additionally be interactive, and may be manipulated in embodiments. In some embodiments, one or more visual overlays include controls for modifying represented oral conditions. In one embodiment, a 3D model of a patient's upper and/or lower patient's dental arches are output to a display by visualization engine 330, and user interface 332 may provide controls for interacting with and/or changing a view of the 3D model(s). As the 3D model(s) are rotated, for example, visualization engine 330 may scroll a panoramic x-ray, or update a highlighted or emphasized region of the panoramic x-ray currently shown in a view of the 3D model(s) to show those teeth that are aligned with current view of the 3D model(s). In some embodiments, visualization engine 330 may determine a bite-wing and/or periapical x-ray that most closely aligns with a current view of the 3D model(s), and may display the determined x-ray(s) in the user interface 332. In some embodiments, multiple views of the patient's dentition are shown together (e.g., in different regions of a display).

Report generation engine 333 may generate reports for patients based on the outputs of segmentation engine 312, oral health diagnostics engine 321 and/or treatment recommendation engine 325. In embodiments, reports can be generated upon a click of a button or other graphical user interface (GUI) element (e.g., via a selection from an element of a menu), an entry from a command line, etc. Once a report generation request is received, report preference information (patient information, doctor preference information, etc.) may be gathered from various databases that store that information. Such preference information may include doctor preferences and/or dental practice preferences. Preference information may include treatment preferences, preferred treatment modalities, preferred views (e.g., panoramic, bitewing, periapical, occlusal, buccal, lingual, etc.), preferred imaging modalities (e.g., radiograph, CBCT, color image, etc.), preferred arrangements of data, and so on. Preference information can be learned based on prior reports generated by and/or for doctors and/or dental practices in embodiments. Doctors and/or dental practices may additionally input their preferences. In some embodiments, requests to generate reports may include one or more parameters for report generation. Such report preference information and/or parameters may be used to configure the report in some embodiments. A request to run a report may trigger one or more report generation processes. Such processes may be independent processes and/or may be dependent on processes of other engines (e.g., visualization engine 330, segmentation engine 312, treatment recommendation engine 325, oral health diagnostics engine 321, and so on).

In some embodiments, report generation engine 333 stores report templates in a data store, and may use such report templates to generate and/or manage reports. In some implementations, report templates include one or more sets of report fields that are to be populated, e.g., at runtime, to generate a report.

A report generation request may, but need not, include report parameters. An example of report parameters that may be specified include limits to the numbers and/or types of oral conditions, actionable recommendations, diagnoses, and/or treatment recommendations in a report. Another example of report parameters that may be specified include limits to report length, format parameters (e.g., colors, fonts, locations of various elements, etc.), locations and/or attributes of interactive elements, security and/or encryption parameters (e.g., anything related to access rights to a report and/or ability to share a report), etc.

Generated reports may include image data from one or more image modalities that were evaluated to generate the outputs. The report may include the image data along with an overlay of one or more identified oral conditions over the image data. Additionally, the report may include a dental chart with oral conditions indicated for teeth having those oral conditions. The report may additionally include a list of oral conditions (e.g., in text form). The reports may be diagnostic data reports that summarize oral conditions detected from relevant oral state capture modalities, actionable symptom recommendations, and/or diagnoses of oral health problems associated with the oral conditions. The report may include treatment recommendations, which may include one more actions to take to effectuate treatment of the oral conditions and/or oral health problems. In embodiments, reports can be generated and/or prioritized based on various factors, such as doctor preferences, issue importance, patient historical factors, etc.

Report generation engine 333 can operate to prioritize diagnostic report elements based on attributes of radiographs and/or other oral state capture modalities, user preferences, relevance of patient information, and/or other information. The report generation engine 333 can use trained models (e.g., ML models) to automatically generate reports in some embodiments. The trained models may be trained for a specific doctor and/or dental practice, and may generate reports in a format preferred by the doctor and/or dental practice. Accordingly, the report generation engine 333 can present issues, actionable recommendations, diagnoses, actions to effectuate treatment, proposed treatments, etc. in a manner preferred by the doctor and/or dental practice.

Generated reports can be presented as a document (e.g., a Word document or PDF document), as a webpage, as a page of an application, and/or in another format. Reports may or may not be interactive. Interactive reports may include interactive elements that a user may interact with to modify the report, provide additional information about aspects of the report, and so on. Reports may be formatted according to preferences of a doctor, dental practice, insurance company, and so on. Information related to report preferences may help to prioritize the data, organize the data, and/or present the data in a diagnostic data report.

The report generation engine 325 may use attributes of detected issues (e.g., oral conditions, actionable symptom recommendations, diagnoses of oral health problems, etc.) and/or report preference information to prioritize identified issues. This may involve AI/ML-based understanding of the types of issues that are likely to be relevant to users and/or specific oral conditions, actionable recommendations, diagnoses, actions to effectuate treatment, and so on. In some implementations, the report generation engine 333 uses one or more AI and/or ML models to intelligently prioritize and display oral conditions, actionable recommendations, diagnoses, and/or treatment recommendations.

The report generation engine 333 can operate to render reports in an interactive manner. Rendering techniques can involve interactive displays where users interact with reports. Alternatively, reports may be static diagnostic reports with text, images/views, treatment options presented to a user. The user interface 332 can operate to facilitate user interactions with diagnostic reports in embodiments.

Once a report is generated, in some embodiments, the report generation engine 333 may receive and/or process user interactions with the generated report based on a user interaction with user interface 332, and provide these user interactions to other systems to update issues related to oral conditions, actionable recommendations, diagnoses, actions to effectuate treatment, etc. In some embodiments, other engines and/or integrated systems may use the reports and/or interactions with them for treatment planning, etc.

One or more data stores 308 may store input data (e.g., data captured using one or more oral state capture modalities) and output data (e.g., reports, determined oral state conditions, diagnoses of oral health problems, actionable symptom recommendations, treatment recommendations, and so on). In some embodiments, treatment recommendations and/or actionable symptom recommendations are stored in a recommendation data store 340, and/or determined oral conditions are stored in oral condition data store 343. Alternatively, the varies types of detections, analysis results, etc. may be stored together in a single data store.

In some embodiments, data store 308 may store captured data from one or more oral state capture modalities, as indicated above, and may include pooled patient data, which may include X-rays, 2D intraoral images, 3D intraoral images, 2D models, and/or virtual 3D models regarding a multitude of patients. Such a multitude of patients may or may not include the at-hand patient. The pooled patient data may be anonymized and/or employed in compliance with regional medical record privacy regulations (e.g., the Health Insurance Portability and Accountability Act (HIPAA)). The pooled patient data may include data corresponding to scanning of the sort discussed herein and/or other data. Reference data may additionally or alternatively include pedagogical patient data, which may include X-rays, 2D intraoral images, 3D intraoral images, 2D models, virtual 3D models, and/or medical illustrations (e.g., medical illustration drawings and/or other images) employed in educational contexts.

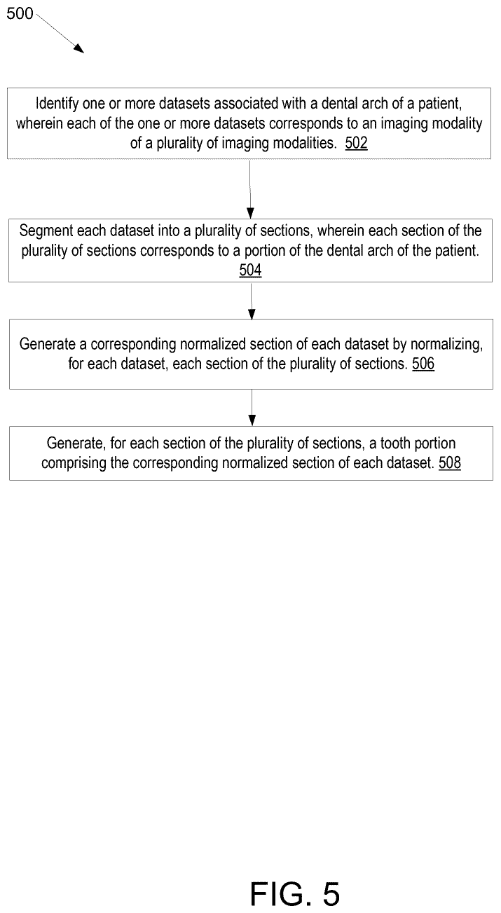

FIG. 4 illustrates a flow diagram of an example method 400 for generating and/or displaying a dynamic tooth chart, in accordance with some embodiments of the present disclosure. FIG. 5 illustrates a flow diagram of an example method 500 for generating a tooth portion of a dynamic tooth chart, in accordance with some embodiments of the present disclosure. One or more of methods 400-500 may be performed by a processing device that may include hardware, software, or a combination of both. The processing device may include one or more central processing units (CPUs), graphics processing units (GPUs), field-programmable gate arrays (FPGAs), application-specific integrated circuits (ASICs), or the like, or any combination thereof. In one embodiment, one or more of methods 400-500 may be performed by the processing devices and the associated algorithms, e.g., as described in conjunction with FIG. 3. In embodiments, one or more of methods 400-500 is performed by processing logic comprising hardware, software, firmware, or a combination thereof. In certain embodiments, one or more of methods 400-500 may be performed by a single processing thread. Alternatively, one or more of methods 400-500 may be performed by two or more processing threads, each thread executing one or more individual functions, routines, subroutines, or operations of the method. In an illustrative example, the processing threads implementing one or more of methods 400-500 may be synchronized (e.g., using semaphores, critical sections, and/or other thread synchronization mechanisms). Alternatively, the processing threads implementing one or more of methods 400-500 may be executed asynchronously with respect to each other. Therefore, while FIGS. 4-5 and the associated descriptions list the operations of methods 400-500 in a certain order, in some embodiments, at least some of the described operations may be performed in parallel and/or in a different order. In some embodiments one or more operations of one or more of methods 400-500 is not performed.

Referring to FIG. 4, at block 402, processing logic receives one or more datasets associated with a patient. Each dataset of the one or more datasets comprises data corresponding to an imaging modality of a plurality of imaging modalities and provides information on a dentition of the patient. The plurality of imaging modalities can include, for example, intraoral scan, near-infrared, cone beam computed tomography (CBCT), photograph, video, radiograph, fluorescence imaging, OCT imaging, and/or any other type of imaging modality. In some embodiments, each dataset can include data from a particular point in time.

In some embodiments, processing logic determines the imaging modality of the plurality of imaging modalities by identifying a correlation between a context of the dental visit and each of the plurality of imaging modalities, ranking each of the imaging modalities according to the correlation, and then determining the imaging modality with the highest ranking. The context of the dental visit can correspond to a complaint of the patient, an input of a user of the user device, a treatment plan, a treatment type, a scheduled dental visit, and/or a detection of a condition. As an illustrative example, a patient may present with a chief complaint of sensitivity in the upper right molar region. Upon receiving this context, the processing logic can analyze the available imaging modalities (e.g., intraoral photographs, bitewing radiographs, panoramic x-rays, and/or NIRI images) and evaluate their respective diagnostic value for detecting caries or other pathology in the specified area that corresponds to the patient's chief complaint. The processing logic can determine a higher correlation score to bitewing radiographs, as this modality is particularly effective for interproximal caries detection in posterior teeth. In contrast, intraoral photographs may receive a lower ranking due to their limited ability to visualize subsurface lesions. Based on this ranking, the processing logic can select the bitewing radiograph as the primary imaging modality for populating the relevant section of the dynamic tooth chart. The selected modality can then be used to generated and display the corresponding tooth portion units. In some embodiments, the processing logic can select the one or more tooth portions that correspond to the determined imaging modality.

In some embodiments, the processing logic can determine the diagnostic value of an imaging modality for a specified area based on a combination of predefined clinical rules, context-aware algorithms, and/or ML models trained on historical diagnostic outcomes. For example, when a clinical context is provided (e.g., a patient's chief complaint, a selected tooth or region, or a specific diagnostic task), the processing logic can reference a knowledge base or decision matrix that encodes the relative effectiveness of each imaging modality for various diagnostic purposes and/or anatomical locations. As an illustrative example, the knowledge base may specify that bitewing radiographs have high diagnostic value for detecting interproximal caries in posterior teeth, while NIRI imaging may be more effective for early enamel lesions on occlusal surfaces.

In some embodiments, the processing logic may further analyze metadata associated with each available image, such as anatomical coverage, imaging quality, and/or recency, to determine whether the modality is relevant and/or suitable for the area of interest. In some embodiments, the processing logic can implement an AI model that is trained to evaluate the likelihood that a given modality will reveal clinically significant findings. In some embodiments, the processing logic can assign a diagnostic value score to each modality (e.g., to each available modality for that patient) by combining these factors (e.g., clinical guidelines, image metadata, and/or AI predictions) and then rank the modalities accordingly. The modality with the highest diagnostic value for the specified area can be selected for visualization and/or further analysis.

At block 404, processing logic processes the one or more datasets to generate, for each of the one or more datasets, one or more tooth portions, wherein each tooth portion comprises an image of one or more teeth of the patient.

In some embodiments, processing logic can normalize the one or more tooth portions. For example, in some embodiments, processing logic can normalize the one or more tooth portions for a size, a scale, a color balance, a brightness, and/or an orientation for coordinated presentation on the dental chart.

At block 406, processing logic arranges the one or more tooth portions into a dental chart.

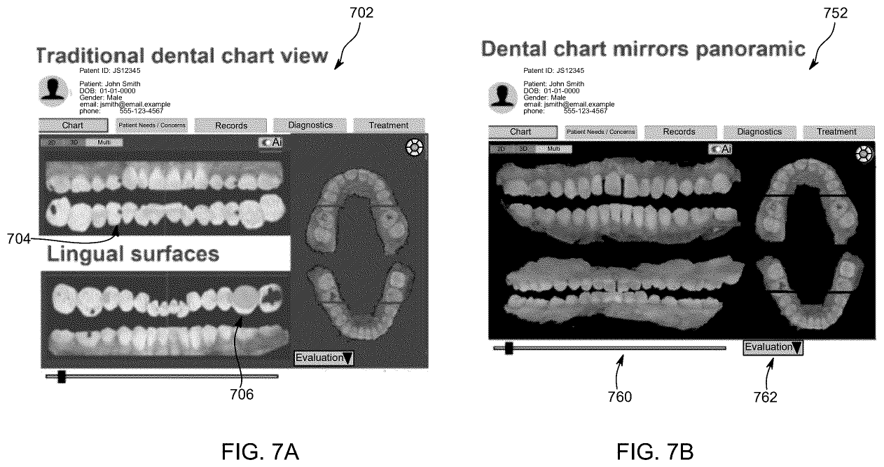

At block 408, processing logic generates a visualization of the dental chart of the patient. In some embodiments, the visualization can include a first portion displaying a dental arch of the patient in a panoramic format and a second portion displaying the dental arch of the patient in an arch format. An example of such a visualization is described with respect to FIG. 7A, B.

In some embodiments, the visualization of the dental chart can include a buccal view, an occlusal view, and/or a lingual view of a dental arch of the patient. An example of the visualization is described with respect to FIG. 6.

In some embodiments, the visualization of the dental chart displays a dental arch of the patient in a two-dimensional view, a three-dimensional view, and/or a multi-dimensional view.

In some embodiments, the visualization can include two or more imaging modalities.

In some embodiments, a first tooth portion of the one or more tooth portions has a first imaging modality and a second tooth portion of the one or more tooth portions has a second imaging modality.

At block 410, processing logic provide, to a user device (e.g., device 360 of FIG. 3), the visualization of the dental chart for presentation in a user interface. In some embodiments, the visualization can correspond to a particular point in time.

In some embodiments, processing logic can receive, from the user device, a user interaction associated with the visualization of the dental chart. Processing logic can identify, based on the user interact, a second imaging modality of the plurality of imaging modalities. Processing logic can update the visualization of the dental chart of the patient by selecting the one or more tooth portions that each corresponds to the second imaging modality. As an illustrative example, after initially displaying the dental chart using bitewing radiographs as the primary imaging modality, a user (e.g., dental practitioner) may interact with the user interface to select a specific tooth and request an alternative view, such as a near-infrared (NIRI) image or a 3D intraoral scan. Upon receiving this user interaction, the processing logic can identify the corresponding tooth portion units available in the selected secondary modality. In some embodiments, the processing logic can dynamically update the chart visualization by replacing the relevant tooth portion or region with the normalized image data from the NIRI or 3D scan, while maintaining the overall chart layout and anatomical alignment.

In some embodiments, processing logic can process the at least one of the one or more datasets using a trained AI model that outputs one or more detected oral conditions. Processing logic can update the one or more tooth portions to include a representation of the one or more detected oral conditions detected for one or more teeth represented in the one or more tooth portions. For example, processing logic can update a corresponding tooth portion to include indications of the findings. In some embodiments, processing logic can update the corresponding tooth unit visualization within the dental chart to visually indicate the findings, e.g., by overlaying color-coded markers or annotations directly onto the affected regions of the tooth images.

In some embodiments, the UI enables a user of the user device to interact with the visualization by focusing in a particular section of a dental arch, changing imaging modalities, and/or changing a point in time. As an illustrative example, a user reviewing a patient's dynamic tooth chart can utilize the UI to zoom in on the lower left quadrant of the dental arch, e.g., where a recent restoration was performed. The UI can provide interactive controls that allow the user to isolate this region, automatically enlarging the relevant tooth portion units and/or suppressing the display of unrelate areas for enhanced clarity. The user can switch the imaging modality, e.g., from a color intraoral scan to a bitewing radiograph, e.g., to assess the integrity of the restoration and/or adjacent interproximal surfaces. As another example, the user can select a previous time point (e.g., the pre-restorative state), enabling side-by-side comparison of the affected teeth before and after treatment.

In some embodiments, processing logic can receive, from the user device, view one or more controls of the UI, a modification to a measurement of a tooth of the one or more teeth of the patient. For example, a user can utilize a digital caliper tool within the UI to adjust the recorded mesiodistal width of a maxillary central incisor, e.g., based on new clinical findings or updated scan data. Processing logic can generate one or more updated tooth portions to reflect the modification. For example, upon receiving the measurement modification, the processing logic can update the corresponding tooth portion unit to reflect the revised dimension, automatically recalibrating the spatial alignment and scaling of adjacent teeth within the dynamic chart. The processing logic can propagate the change to related clinical records, such as orthodontic treatment plans or prosthetic design files. Processing logic can generate an updated visualization of the dental chart to include the one or more updated tooth portions, and can provide, to the user device, the updated visualization of the dental chart.

For example, a user may use the UI to increase the recorded incisal edge length of a maxillary lateral incisor, reflecting a planned restorative procedure or a correction based on new scan data. Upon receiving a measurement modification, the processing logic can recalculate the geometry of the affected tooth portion unit, and ca update the visualization to depict the new crown height, for example. The processing logic can regenerate the visualization of the dental chart, integrated the updated tooth portion into the overall chart layout while maintaining anatomical alignment and proportional relationships with adjacent teeth. The updated chart can be provided to the user, allowing the user to assess the esthetic ad/or functional impact of the modification from multiple perspectives, such as buccal, lingual, and/or occlusal views.

In some embodiments, in orthodontic treatment planning, precise measurement and visualization of the proportional relationships between the heights and widths of anterior teeth (e.g., central incisors, lateral incisors, and canines) affect esthetic and functional outcomes. For example, the height-to-width ratio of the maxillary central incisor can be targeted at approximately 80%. As another example, the lateral incisor width can be targeted to be approximately 78% of the central incisor width. The dynamic tooth charting system can enable users to visualize these ratios directly within the chart interface, with real-time overlays or measurement tools that display the current dimensions and proportional relationships of each tooth. Processing logic can provide synchronized, multi-angle visualizations (e.g., buccal, occlusal, and/or lingual views), allowing a user to assess the effects of tooth movements and dimensional changes for all relevant perspectives simultaneously. As the user modifies the position and/or size of a tooth, processing logic can automatically recalculate the ratios and display the updated ratios, highlight deviations from targeted proportions, and/or provide visual cues to guide further adjustments.

In some embodiments, processing logic can provide, as input, the one or more datasets to an AI model that is trained to provide an indication of a clinical finding corresponding to a particular imaging modality of the plurality of imaging modalities. Processing logic can receive, from the AI model, the indication of the clinical finding corresponding to the particular imaging modality. Processing logic can include the clinical finding in the visualization of the dental chart of the patient. In some embodiments, to include the clinical finding in the visualization of the dental chart, processing logic can determine a second imaging modality of the plurality of imaging modalities other than the particular imaging modality, and can select the one or more tooth portions corresponding to the second imaging modality. In some embodiments, to determine the second imaging modality, processing logic can identify a correlation between the clinical finding and each of the plurality of imaging modalities. Processing logic can rank each of the plurality of imaging modalities according to the correlation, and can determine the second imaging modality as the imaging modality with the highest ranking.

In some embodiments, processing logic can receive, from at least one AI model, a plurality of indications of a clinical finding. Each indication corresponds to a particular imaging modality of the plurality of imaging modalities. Processing logic can identify a primary imaging modality corresponding to the clinical finding. Processing logic can identify a primary indication of the plurality of indications. The primary indication corresponds to the primary imaging modality. Processing logic can include the primary indication in the visualization of the dental chart of the patient.

In some embodiments, processing logic can generate an overall indication of the clinical finding by aggregating the plurality of indications of the clinical finding. Processing logic can include the overall indication of the clinical finding in the visualization of the dental arch of the patient.

As an illustrative example, one indication of a clinical finding can correspond to a first imaging modality. For example, the first imaging modality can indicate a caries associated with a tooth. Another indication corresponding to a second imaging modality may not indicate the carries associated with that tooth (e.g., the tooth is hidden behind an overlapping tooth in a bite wing). To reconcile the conflicting AI generated data (e.g., to reconcile the two conflicting indications of the clinical finding), processing logic can identify a hierarchy of accuracy of imaging modalities corresponding to particular clinical findings. For example, the first imaging modality may have a higher accuracy for that particular clinical finding (e.g., carries) than the second imaging modality. Processing logic can then identify the indication associated with the imaging modality with the higher accuracy. In some embodiments, processing logic can combine the conflicting indications associated with the imaging modalities. For example, the first indication of the clinical finding corresponding to the first imaging modality can have a first confidence value (e.g., 0.9 likelihood that the caries is present), and the second indication of the clinical finding corresponding to the second imaging modality can have a second confidence value (e.g., 0.2 likelihood that the caries is present), the processing logic can combine the two confidence levels to generate an overall indication of the clinical finding. For example, processing logic can take the average of the confidence values (e.g., to continue the illustrative example above, (0.9+0.2)÷2=0.55) to indicate the presence of the clinical finding.

In some embodiments, the data corresponding to the imaging modality comprises image data associated with a plurality of image sources. Processing logic can provide, as input, the image data associated with the plurality of image sources to an AI model trained to provide a confidence score associated with an identified clinical finding for each of the plurality of image sources. Processing logic can receive, as output from the AI model, a plurality of confidence scores associated with a clinical finding for the plurality of image sources. Processing logic can identify an image source of the plurality of image sources with the highest confidence score, and can include, in the visualization of the dental chart, the image source with the highest confidence score.

In embodiments, processing logic can use one or more machine learning models in the generation of the visualization of the dental chart of the patient. One type of machine learning model that may be used is an artificial neural network, such as a deep neural network. Artificial neural networks generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g. classification outputs). Deep learning is a class of machine learning algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. In an image recognition application, for example, the raw input may be a matrix of pixels; the first representational layer may abstract the pixels and encode edges; the second layer may compose and encode arrangements of edges; the third layer may encode higher level shapes (e.g., teeth, gingiva, enamel, etc.); and the fourth layer may recognize that the image contains a face or define a bounding box around teeth in the image. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available.

In some embodiments, the training dataset can contain hundreds, thousands, tens of thousands, hundreds of thousands, or more images (e.g., scan data, video data, and/or additional patient data) may be provided. In some embodiments, the training dataset can include labeled 3D color models generated from intraoral scan data of the dentition of a patient and/or color 2D images. In some embodiments, some or all of the data may be labeled with segmentation information, oral condition information, clinical finding information, and/or other information.

In some embodiments, some or all of the image-based data in the training dataset can be processed by a segmenter that segments the image-based data input individual teeth, and that outputs segmentation information (e.g., stored in segmentation data 353 of FIG. 3). The segmenter may be or include, for example, a trained machine learning model such as a convolutional neural network (CNN) trained to classify pixels or regions of input images into different classes. This can include performing point-level classification (e.g., pixel-level classification or voxel-level classification) of different types of features and/or objects of subjects of images. The different features and/or objects may include, for example, mandible, teeth, the TMJ's cartilage disc, etc. The segmenter may output one or more masks, each of which may have a same resolution as an input image. The mask or masks may include a different identifier for each identified feature or object, and may assign the identifiers on a pixel-level or patch-level basis. In one embodiment, different masks are generated for one or more different classes of features and/or objects. In one embodiment, a single mask or map includes segmentation information for all identified classes of features and/or objects. Some types of features are location-specific features and are represented in one or more masks.

In some embodiments, the segmenter performs one or more image processing and/or computer vision techniques or operations to extract segmentation information from images. Such image processing and/or computer vision techniques may or may not include the use trained machine learning models. Accordingly, in some embodiments, segmenter does not include a machine learning model.

In some embodiments, the training dataset, and optionally the segmentation information (e.g., segmentation data 353) can be used to train one or more machine learning models to indicate clinical findings, oral conditions, treatment plans, primary imaging modalities, and/or primary image sources corresponding to an imaging modality. Training may be performed by inputting one or more data points and optionally corresponding segmentation information into the machine learning model one at a time. The data that is input into the machine learning model may include a single layer or multiple layers. In some embodiments, a recurrent neural network (RNN) is used. In such an embodiment, a second layer may include a previous output of the machine learning model (which resulted from processing a previous input).

The machine learning model processes the input to generate an output. An artificial neural network includes an input layer that consists of values in a data point. The next layer is called a hidden layer, and nodes at the hidden layer each receive one or more of the input values. Each node contains parameters (e.g., weights) to apply to the input values. Each node therefore essentially inputs the input values into a multivariate function (e.g., a non-linear mathematical transformation) to produce an output value. A next layer may be another hidden layer or an output layer. In either case, the nodes at the next layer receive the output values from the nodes at the previous layer, and each node applies weights to those values and then generates its own output value. This may be performed at each layer. A final layer is the output layer, where there is one node for each class, prediction and/or output that the machine learning model can produce.

Processing logic may then compare the generated values and/or categorizations to the known condition and/or label that was included in the training data item. Processing logic determines an error based on the differences between the output probability map and/or label(s) and the provided probability map and/or label(s). Processing logic adjusts weights of one or more nodes in the machine learning model based on the error. An error term or delta may be determined for each node in the artificial neural network. Based on this error, the artificial neural network adjusts one or more of its parameters for one or more of its nodes (the weights for one or more inputs of a node). Parameters may be updated in a back propagation manner, such that nodes at a highest layer are updated first, followed by nodes at a next layer, and so on. An artificial neural network contains multiple layers of “neurons,” where each layer receives input values from neurons at a previous layer. The parameters for each neuron include weights associated with the values that are received from each of the neurons at a previous layer. Accordingly, adjusting the parameters may include adjusting the weights assigned to each of the inputs for one or more neurons at one or more layers in the artificial neural network.

Once the model parameters have been optimized, model validation may be performed to determine whether the model has improved and to determine a current accuracy of the model. After one or more rounds of training, processing logic may determine whether a stopping criterion has been met. A stopping criterion may be a target level of accuracy, a target number of processed data items from the training dataset, a target amount of change to parameters over one or more previous data points, a combination thereof and/or other criteria. In one embodiment, the stopping criteria is met when at least a minimum number of data points have been processed and at least a threshold accuracy is achieved. The threshold accuracy may be, for example, 70%, 80% or 90% accuracy. In one embodiment, the stopping criteria is met if accuracy of the machine learning model has stopped improving. If the stopping criterion has not been met, further training is performed. If the stopping criterion has been met, training may be complete. Once the machine learning model is trained, a reserved portion of the training dataset (and optionally segmentation information) may be used to test the model. Testing the model can include performing unit tests, regression tests, and/or integration tests.

Referring to FIG. 5, at block 502, processing logic identifies one or more datasets associated with a dental arch of a patient. Each dataset corresponds to an imaging modality of a plurality of imaging modalities. The plurality of imaging modalities can include, for example, intraoral scan, near-infrared, CBCT, photograph, video, radiograph, fluorescence, OCT, and/or any other type of imaging modality. In some embodiments, a dataset of the one or more datasets can correspond to a buccal view, an occlusal view, or a lingual view of the dental arch of the patient.

At block 504, processing logic segments each dataset into a plurality of sections. Each section corresponds to a portion of the dental arch of the patient. A portion of the dental arch can correspond to one or more teeth of the dental arch of the patient.

At block 506, processing logic generates a corresponding normalized section of each dataset by normalizing, for each dataset, each section of the plurality of sections. In some embodiments, each section can be normalized for size, scale, color balance, and/or orientation.

At block 508, processing logic generates, for each section of the plurality of sections, a tooth portion comprising the corresponding normalized section of each dataset.

FIG. 6 illustrates an example user interface 600 displaying a visualization of a dynamic tooth chart 602, in accordance with some embodiments of the present disclosure. As illustrated in FIG. 6, the UI 600 can include patient information 610 (e.g., corresponding to patient data 356 of FIG. 3), and can provide a variety of tool options for the user. The tool options include viewing the dental chart 612, viewing patient needs/concerns 614, viewing patient records 616, viewing diagnostics 618, and viewing treatments 620. The visualization of the dental chart 602 can be displayed in 2D (630), 3D (632), or multi-view (634). The embodiment displayed in FIG. 6 illustrates the dynamic tooth chart in 3D format. The visualization of the dynamic tooth chart 600 also provides an AI toggle 640. The AI toggle 640 can enable the user to include or exclude indications of AI identified clinical findings or conditions.

In some embodiments, the 2D visualization mode (e.g., corresponding to 2D UI element 630) can present the dental chart with teeth arranged in a linear or arch-shaped layout. In some embodiments, the 2D visualization can include buccal views, occlusal views, and/or lingual views of the dental arch. In some embodiments, in the 2D mode, tooth portions can be displayed as normalized images extracted from various imaging modalities, such as intraoral scans, radiographs, photographs, and/or NIRI images. In some embodiments, the 2D visualization can include chart annotations, clinical findings, and/or tooth numbering overlaid directly on the tooth images

In some embodiments, the 3D visualization mode (e.g., corresponding to the 3D UI element 632) can provide a three-dimensional representation of the patient's dental arch based on 3D model data generated from intraoral scans. In some embodiments, the 3D mode can display the dental chart using the 3D model that has been segmented to identify individual teeth and their spatial context. In some embodiments, a user can view teeth from multiple perspectives, such as buccal, lingual, and/or occlusal views. In some embodiments, the 3D rendering can display tooth shape and/or geometry of individual teeth. In some embodiments, the 3D mode can distinguish itself from the 2D mode by enabling visualization of spatial relationships between teeth that may not be visible in flat 2D representations. In some embodiments, the 3D visualization can display tooth orientation relative to the jawline reference curve.

In some embodiments, the multi-view or multi-dimensional mode (e.g., corresponding to UI element 634) can encompass display of data from multiple imaging modalities within a single interface. In some embodiments, the multi-view mode can present the dental arch from different anatomical views, such as buccal, occlusal, and/or lingual views. In some embodiments, the multi-view mode can utilize data from multiple imaging modalities, such as a 3D model, 2D color images, and/or NIRI images, with corresponding anatomical features aligned across each modality based on geometric connections between the 3D and 2D data. In some embodiments, the multi-dimensional mode can include visualizations where segmentation information and/or clinical findings are overlaid on the underlying imaging data. The multi-view mode can facilitate comprehensive assessment by allowing dental practitioners to correlate findings across different imaging modalities and anatomical views.

The visualization of the dental chart 602 includes a buccal view, a lingual view, and an occlusal view of the dental arch of the patient. The visualization of the dental chart 602 includes a first portion that displays the dental arch of the patient in a panoramic format and second portion that displays the dental arch of the patient in an arch format.

FIG. 7A illustrates an example user interface 700 displaying a visualization of a dynamic tooth chart 702 in a traditional dental chart view, in accordance with some embodiments of the present disclosure. FIG. 7B illustrates an example user interface 750 displaying a visualization of a dynamic tooth chart 752 that mirrors an panoramic view of the dental arches, in accordance with some embodiments of the present disclosure. In embodiments, tooth numbering, charting, AI detected clinical findings, and/or suggested treatment can be displayed directly on the patient's imaging. For example, visualization 702 includes indications of AI detected clinical findings 704, 706. As illustrated in FIGS. 7A-B, in embodiments, the left-hand side of the UI can display the dental arch in a panoramic view, and the right-hand side of the UI can display the dental arch in an arch format.

In some embodiments, user interfaces 702, 752 can also include a variety of tools that enable the user to interact with the dynamic tooth chart. For example, as illustrated in FIGS. 7A-B, the UI 702,752 can include a slider 760 that allows movements from the most comprehensive preset view to the most limited problem-focused view. The UI 702,752 can include an evaluation dropdown 762 that provides a series of selections that enable a user change the focus of the slider 760 and/or edit preset views. The preset views can be adjustable based on the user preference. In some embodiments, a user can change the focus of the slider 760, e.g., for charting, treatment planning, patient education, evaluation of remotely monitored data, and/or simulation purposes.

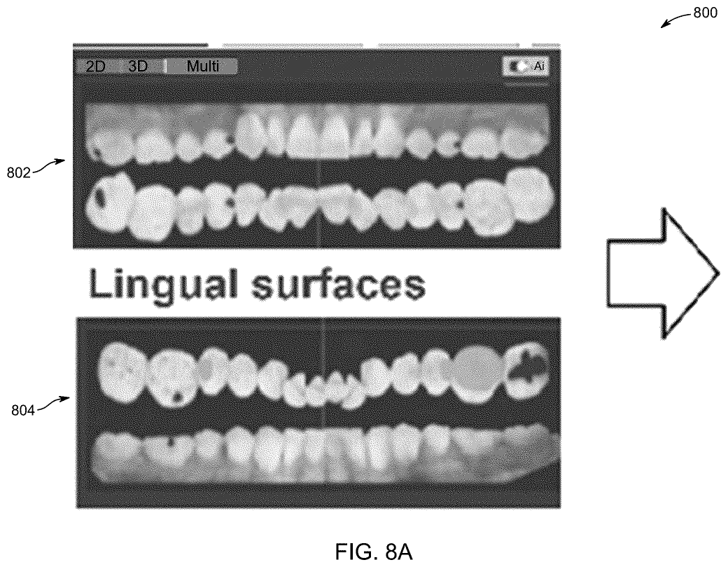

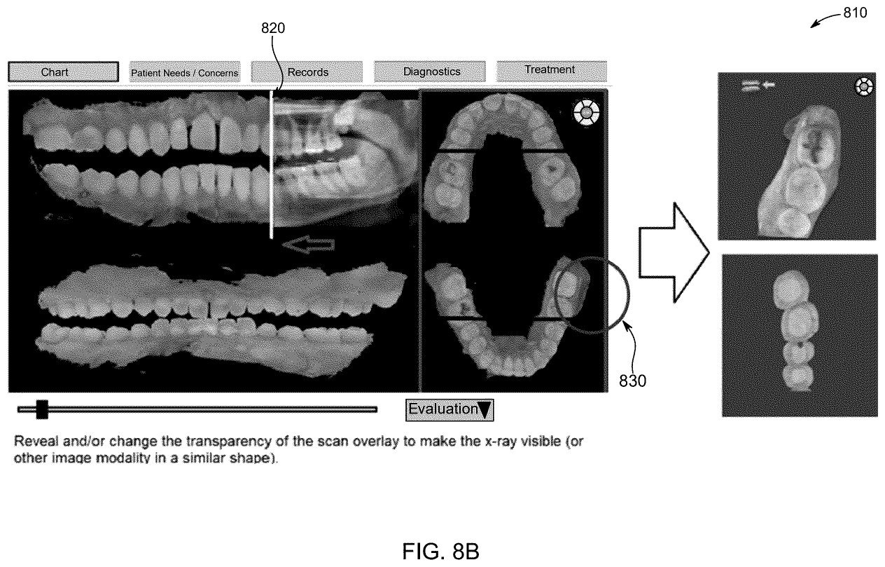

FIGS. 8A-C illustrate examples of visualizations of a dynamic tooth chart using various imaging modalities, in accordance with some embodiments of the present disclosure. As an illustrative example, the visualization 800 of FIG. 8A can be the primary view of the visualization of the dynamic tooth chart. In some embodiments, visualization 800 can correspond to visualization 602 of FIG. 6. FIG. 8A illustrates upper arches 802 and lower arches 804 displayed in a consistent orientation and scale, with tooth portions normalized and aligned to the chart grid to present a coherent, full-mouth view. Visualization 800 reflects a default modality selection, such as an intraoral scan or other baseline imaging modality, projected into the chart format to provide a comprehensive overview. As illustrated in FIG. 8A, the visualization 800 of the dental chart can include indications of identified clinical findings.

FIG. 8B illustrates how a user can change the imaging modality used in the visualization. As an illustrative example, as illustrated in FIG. 8B, the UI 810 can include a slider tool 820 that enables the user to reveal and/or change the transparency of the scan overlay to make the x-ray visible (or other image modality in a similar shape). As illustrated in FIG. 6B, the imaging modality displayed within the dynamic tooth chart can be transitioned to expose a radiographic representation while preserving the standardized chart layout and alignment of tooth portion units. As illustrated in FIG. 6B, a control 820 (e.g., a slider or opacity adjustment) can be used to reveal an x-ray layer beneath a scan-based overlay so that corresponding anatomical features remain co-registered across modalities. In some embodiments, this reveal operation can be performed within the same spatial frame used in FIG. 6A, such that the normalized size, scale, orientation, and/or brightness of each tooth portion continue to snap into the designated chart gird positions. As the user adjusts the transparency, the dynamic chart system can maintain tooth-to-tooth correspondence and chart fidelity, enabling localized inspection of a selected tooth or region of interest without disrupting the overall chart context. In some embodiments, as illustrated in FIG. 8B, a user can focus in on a particular tooth or tooth portion 830.

FIG. 8C illustrates a composite visualization 850 in which multiple imaging modalities are concurrently presented in the dynamic tooth chart to provide corroborative views of the same dentition within the standardized layout. Visualization 850 includes an x-ray view 855, a CBCT scan 856, a NIRI image 857, an intraoral scan images 858. In some embodiments, a midline reference 859 can extend through the occlusal views of scan image 858 and 856, representing the dental midline of the patient's dental arches. The midline reference 859 can serve as a visual guide for assessing symmetry and/or alignment of the dental arches, in embodiments. In some embodiments, the midline reference 859 can be used to evaluate the relationship between the upper and lower arches and/or to assess the alignment of the anterior teeth relative to the facial midline. The midline reference 859 can facilitate treatment planning by providing a reference point for evaluating proposed tooth movements and their effect on overall dental symmetry, in embodiments. As illustrated in FIG. 8C, tooth portion units represented different modalities are arranged in modality-specific layers or adjacent segments while remaining registered to the same chart coordinates, thereby enabling direct comparison across views. In some embodiments, the dynamic chart system can preserve inter-modal alignment by applying previously generated normalization and registration transforms, so that corresponding structures can be consistently located across the composite display.

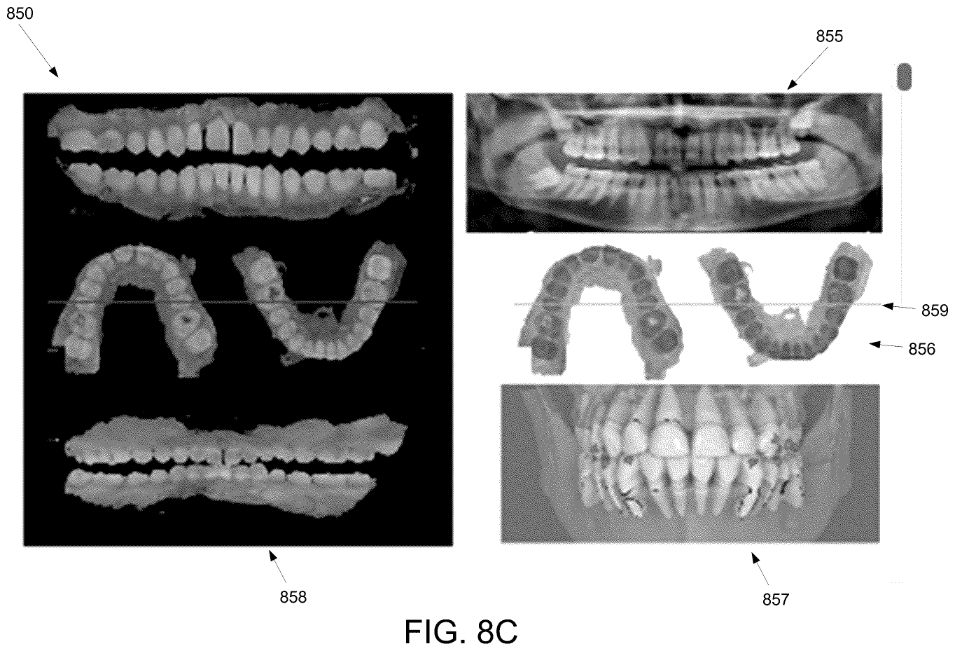

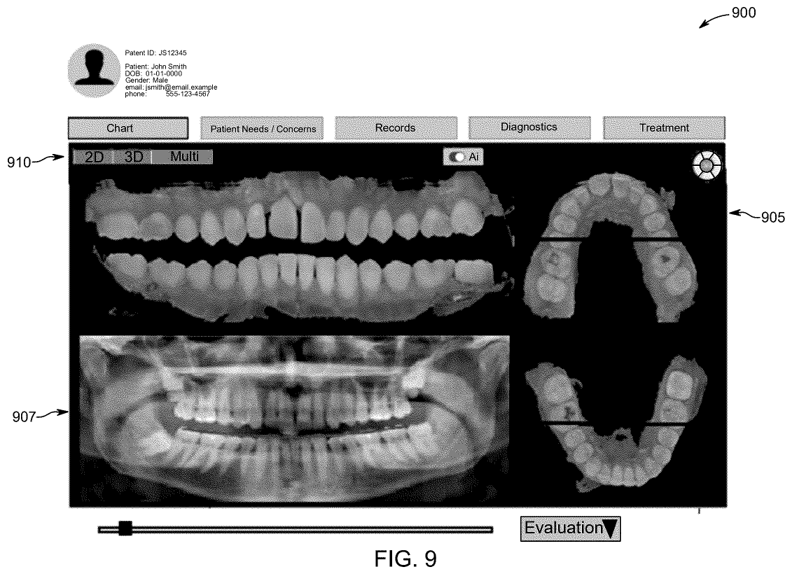

FIG. 9 illustrates an example UI portion 900 displaying the visualization of the patient's dental arches in multiple imaging modalities simultaneously, in accordance with some embodiments of the present disclosure. As illustrated in FIG. 9, the UI portion 900 presents parallel views of the same dental region, such as the upper and lower arches, with each view corresponding to a distinct imaging modality (e.g., intraoral scan 905, x-ray 907, and/or NIRI (not pictured)). Each modality-specific visualization is normalized and registered to a common chart grid so that anatomical features and tooth positions can be aligned across multiple displayed modalities. This configuration can enable a dental practitioner to directly compared and correlate findings between different imaging sources within a single, integrated interface. The dynamic chart system can provide interactive controls for toggling between modalities (e.g., menu 910), synchronizing navigation across views, and/or focusing on specific teeth or regions of interest.

FIG. 10 illustrates an example UI portion 1000 displaying one or more visualizations of the patient's dental arches in multiple imaging modalities displayed in conjunction with two photographs 1010, 1012 of the patient, in accordance with some embodiments of the present disclosure. In an illustrative example, the UI portion 1000 integrates intraoral chart view (e.g., 2D and/or 3D representations of the dental charts, normalized and aligned according to the chart grid, as described with respect to FIG. 8C) with one or more facial photographs or profile images. For example, UI portion 1000 can include scan images 1058 (e.g., corresponding to scan image 858 of FIG. 8C), x-ray view 1055 (e.g., corresponding to x-ray view 855 of FIG. 8C), CBCT scan 1056 (e.g., corresponding to CBCT scan 856 of FIG. 8C), and NIRI image 1057 (e.g., corresponding to NIRI image 857 of FIG. 8C). Each photograph 1010, 1012 in the UI portion 1000 can display a result of different suggested treatment plan. For example, photograph 1010 can display a photograph visualization of the patient's teeth as a result of treatment plan A, and photograph 1012 can display a photograph visualization of the patient's teeth as a result of treatment plan B. The photograph visualizations can be generated by dynamic chart system 315. In some embodiments, the treatment plans A, B can be generated by an AI model. This configuration can enable a dental practitioner to correlate intraoral findings with extraoral anatomical features, supporting comprehensive assessment and treatment planning.

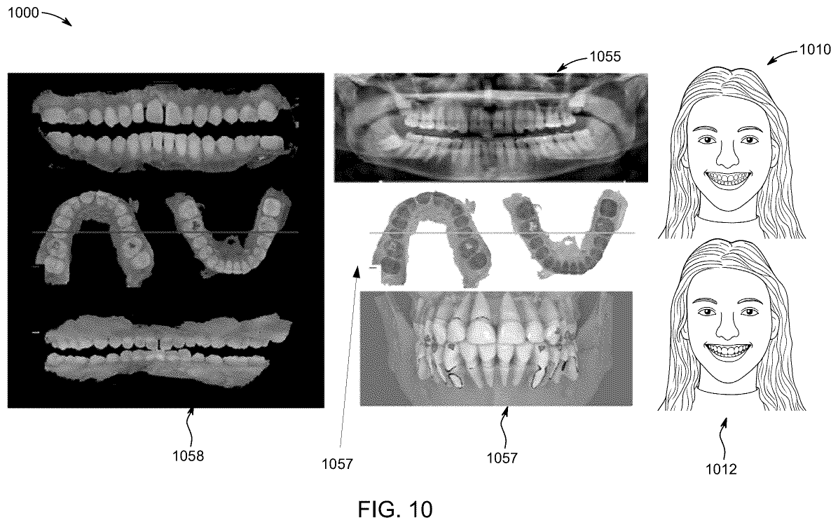

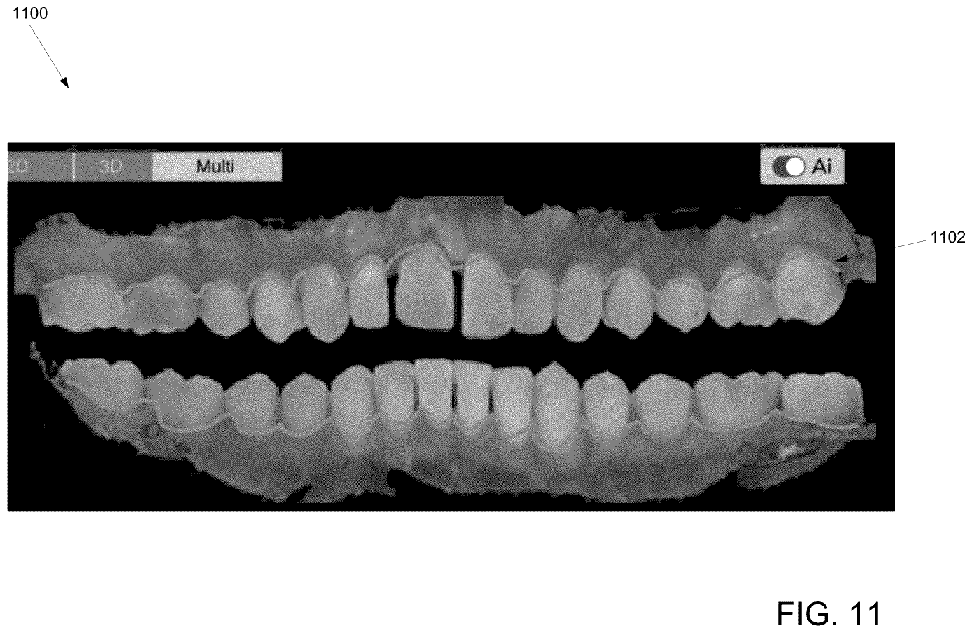

FIG. 11 illustrates an example visualization 1100 of a dental chart in which periodontal data is projected onto an imaging modality, in accordance with embodiments of the present disclosure. In some embodiments, periodontal data from any source can be projected onto any imaging modality. The visualization portion 1100 can display patient-specific imaging (e.g., an intraoral scan or radiograph) serving as the background layer, while periodontal measurements and/or clinical findings can be overlaid directly onto the corresponding anatomical regions. The periodontal data can be, for example, from a dental practice management system, manual input, photo/video analysis, CBCT, radiographic analysis (e.g., using AI or ML), etc. The periodontal data can include probings, gingival recession, bleeding points, furcation involvement, gingival defects, mobility, mucogingival junction, and/or others. In some embodiments, the periodontal data can be spatially registered and visually integrated with the underlying imaging, allowing dental practitioners to assess both hard and soft tissue conditions in a unified view.

As an illustrative example, FIG. 11 illustrates a gingival margin line 1102 the represents the boundary between the gingival tissue and the tooth surface. The gingival margin line 1102 delineates the interface where the gingiva meets the clinical crown of each tooth. The gingival margin line 1102 can serve as a reference for projecting periodontal data onto the imaging modality, in embodiments. In some embodiments, periodontal measurements such as probing depths, gingival recession values, and/or bleeding points can be displayed relative to the gingival margin line 1102. In some embodiments, the gingival margin line 1102 can be automatically detected through segmentation of the 3D model and/or can be manually adjusted by a dental practitioner. In some embodiments, the gingival margin line 1102 can be used to calculate and display gingival recession measurements by comparing the current gingival position to an expected or historical reference position.

FIG. 12 illustrates a flow diagram of an example data flow 1200 for an automated dental charting system, in accordance with some embodiments of the present disclosure. In some embodiments, data flow 1200 may be correspond to a method performed by a processing device that may include hardware, software, or a combination of both. The processing device may include one or more central processing units (CPUs), graphics processing units (GPUs), field-programmable gate arrays (FPGAs), application-specific integrated circuits (ASICs), or the like, or any combination thereof. In one embodiment, data flow 1200 may correspond to a method that may be performed by the processing devices and the associated algorithms, e.g., as described in conjunction with FIG. 3. In embodiments, data flow 1200 may correspond to a method that is performed by processing logic comprising hardware, software, firmware, or a combination thereof. In certain embodiments, the method may be performed by a single processing thread. Alternatively, the method may be performed by two or more processing threads, each thread executing one or more individual functions, routines, subroutines, or operations of the method. In an illustrative example, the processing threads implementing the method may be synchronized (e.g., using semaphores, critical sections, and/or other thread synchronization mechanisms). Alternatively, the processing threads implementing the method may be executed asynchronously with respect to each other. Therefore, while FIG. 12 and the associated descriptions list the operations of data flow 1200 in a certain order, in some embodiments, at least some of the described operations may be performed in parallel and/or in a different order. In some embodiments one or more operations of data flow 1200 is not performed.

In some embodiments, at block 1210, processing logic can identify or receive a 3D model of a patient's jaw, e.g., generated from intraoral scan data acquired by an intraoral scanner. The 3D jaw model can include a dense point cloud or mesh representation that captures the anatomical geometry of both the maxillary and mandibular arches, including individual teeth, gingival tissues, and relevant oral structures.

In some embodiments, at block 1212, processing logic can perform semantic segmentation of the received 3D model, e.g., to delineate and classify distinct anatomical regions within the oral cavity. In some embodiments, the segmentation process can implement one or more machine learning algorithms, such as point cloud-based neural networks (e.g., PointNet++ or sparse convolutional neural networks), to assign semantic labels to each point or mesh vertex in the 3D dataset. The segmentation output distinguishes between hard tissues (individual teeth) and soft tissues (gingiva, palate, tongue), enabling the isolation of each tooth as a discrete secondary 3D model. In some embodiments, the segmentation process assigns tooth identifiers (e.g., FDI or Universal numbering) based on spatial arrangement and morphological features. In some embodiments, at block 1214, processing logic can store the secondary 3D models, each corresponding to a particular tooth, in a data store (e.g., data store 308 of FIG. 3).

In some embodiments, at block 1216, processing logic can perform a teeth arrangement analysis, e.g., as described with respect to teeth arrangement analysis engine 327 of FIG. 3. A result of the teeth arrangement analysis can be an identification of a missing tooth and/or of an irregular tooth. In some embodiments, at block 1216, processing logic executes a teeth arrangement analysis on the segmented 3D jaw model(s) to evaluate the spatial positioning and alignment of individual teeth relative to the anatomical jaw arch. This analysis can involve modeling the jawline reference curve to establish a baseline for expected tooth positions within the dental arch. The processing logic computes the relative translation and orientation of each segmented tooth with respect to this reference curve, enabling the detection of positional anomalies such as crowding, spacing, rotation, or tipping. In some embodiments, the processing logic identifies missing teeth by detecting gaps in the expected tooth sequence and flags irregularities such as ectopic eruptions or transpositions. The arrangement analysis may incorporate occlusal relationships and arch symmetry metrics to further characterize malocclusions or arch form deviations. The processing logic can encode these spatial assessments chart annotations. At block 1218, processing logic can store the spatial assessments (e.g., identification(s) of missing tooth (or teeth) and/or of irregular tooth (or teeth)), e.g., in data store 308 of FIG. 3.

In some embodiments, at block 1220, processing logic can perform a 2D color analysis, e.g., using the output of segmentation 1212. In some embodiments, the processing logic can perform a 2D color analysis by mapping the segmented 3D tooth models onto corresponding 2D color intraoral images, e.g., acquired during the scanning process. The processing logic can utilize geometric calibration data to achieve accurate 3D-to-2D back-projection, so that each region of the 3D model is aligned with its representation in the 2D color images. The processing logic can apply image processing and/or machine learning techniques (e.g., convolutional neural networks (CNNs) or U-Net architectures) to analyze the color and texture features within each tooth region. As a result of the image processing and/or ML model processing, the processing logic can identify and/or segment restorative materials (e.g., composite resin, amalgam, ceramic), detect of surface-level conditions (such as staining, demineralization, or enamel hypoplasia), and/or differentiate between natural tooth structure and artificial restorations. The processing logic can localize findings to specific dental surfaces (e.g., buccal, lingual, occlusal) using standard dental nomenclature. In some embodiments, the processing logic can encode the resulting material and condition classifications as chart annotations.

In some embodiments, at block 1240, processing logic can perform a material segmentation, e.g., as described with respect to material analysis engine 326 of FIG. 3. In some embodiments, at block 1240, processing logic can conduct a material analysis to classify and/or delineate the various dental materials present within each segmented tooth region. The processing logic can integrate multimodal data by projecting the 3D tooth segmentation onto both 2D color and near-infrared imaging (NIRI) intraoral images, leveraging the distinct optical signatures of different restorative and natural materials. In some embodiments, the processing logic can implement image segmentation algorithms, such as deep convolutional neural networks or U-Net models, to distinguish between natural enamel, dentin, and a range of restorative materials including composite resin, amalgam, ceramic, porcelain, and/or metallic alloys. The processing logic can identify the spatial extent and precise location of these materials, enabling the detection and annotation of full-coverage restorations (e.g., crowns, bridges), e.g., at block 1244, and/or partial restorations (e.g., inlays, onlays, veneers), and significant fillings, e.g., at block 1242. In some embodiments, material segmentation may also flag the presence of dental prosthetics such as abutments, scan bodies, or implant-supported crowns, as well as dental accessories like orthodontic brackets or aligner attachments. In some embodiments, the processing logic can encode the resulting material classification and segmentation data as detailed chart annotations, specifying both the type and distribution of materials on each dental surface. For example, the result of the material segmentation can be an identification of a partial restoration, a veneer, an inlay, an onlay, an overlay, and/or a significant filling, and the processing logic can store the identification, e.g., in data store 308 of FIG. 3. For example, the result of the material segmentation can an identification of a full restoration, a crown, and/or a bridge (e.g., as opposed to a natural tooth), and the processing logic can store the identification, e.g., in data store 308 of FIG. 3.

In some embodiments, at block 1222, processing logic can perform a 3D shape analysis, e.g., as described with respect to shape analysis engine 328 of FIG. 3. The result of the 3D shape analysis can be, for example, an indication of a tooth identifier (e.g., a tooth number), an indication of a tooth shape (e.g., an outlier shape), and/or an indication of a dental accessory (or multiple dental accessories). The processing logic can store the tooth ID indicator 1224, the tooth shape indicator 1226, and/or the dental accessory indicator 1228, e.g., in data store 308 of FIG. 3. In some embodiments, the tooth ID indicator 1224 and/or the shape indicator 1226 can be provided to a classification model at block 1230. The dental accessory 1228 can be provided to a detection model at block 1236.

In some embodiments, at block 1230, processing logic can generate clinically relevant classifications. In some embodiments, processing logic can implement geometric processing algorithms and/or ML models to analyze the spatial structure and/or surface topology of each tooth, e.g., as determined at block 1224 and/or block 1226. For example, the analysis can include quantifying parameters such as crown height, cusp configuration, axial inclination, and/or overall volumetric geometry. These parameters can be compared against normative dental templates and/or feature embeddings to determine the specific tooth ID. The processing logic can detect anomalies, such as partially erupted, fractured, and/or malformed teeth. The classification can result in an accurate tooth identification, and an indication of whether the tooth is a primary tooth, e.g., at block 1232. The classification can result in an indication of an abutment, a scan-body, a prep, a partially hatched tooth, and so on, e.g., at block 1234.

In some embodiments, at block 1236, the processing logic can assess deviations from standard morphology to identify the presence of dental accessories or non-natural structures, such as orthodontic brackets, aligner attachments, abutments, scan bodies, and/or prepped teeth. Upon identifying the presence of a dental accessory, the processing logic can store corresponding indicators of brackets, attachments, retainers, braces, etc., e.g., at block 1238.

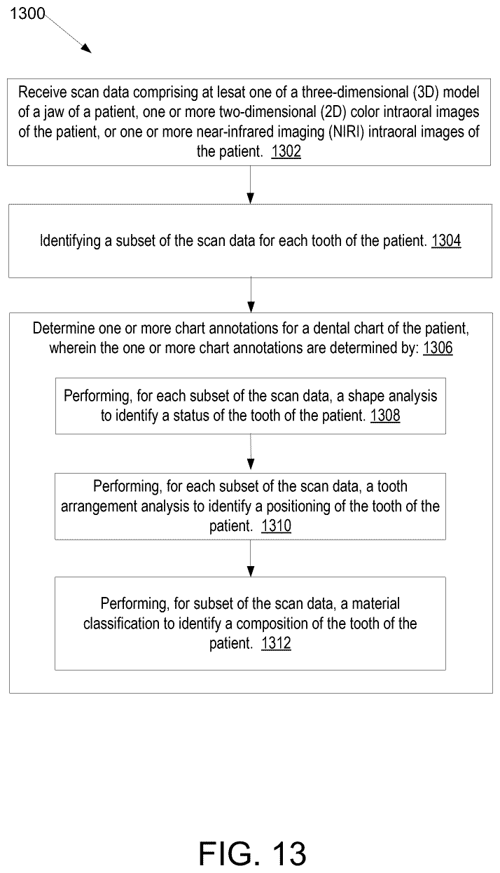

FIG. 13 illustrates a flow diagram of an example method 1300 for automatically generating tooth annotations for a dental chart, in accordance with some embodiments of the present disclosure. Method 1300 may be performed by a processing device that may include hardware, software, or a combination of both. The processing device may include one or more central processing units (CPUs), graphics processing units (GPUs), field-programmable gate arrays (FPGAs), application-specific integrated circuits (ASICs), or the like, or any combination thereof. In one embodiment, method 1300 may be performed by the processing devices and the associated algorithms, e.g., as described in conjunction with FIG. 3. In embodiments, method 1300 is performed by processing logic comprising hardware, software, firmware, or a combination thereof. In certain embodiments, method 1300 may be performed by a single processing thread. Alternatively, method 1300 may be performed by two or more processing threads, each thread executing one or more individual functions, routines, subroutines, or operations of the method. In an illustrative example, the processing threads implementing method 1300 may be synchronized (e.g., using semaphores, critical sections, and/or other thread synchronization mechanisms). Alternatively, the processing threads implementing method 1300 may be executed asynchronously with respect to each other. Therefore, while FIG. 13 and the associated descriptions list the operations of method 1300 in a certain order, in some embodiments, at least some of the described operations may be performed in parallel and/or in a different order. In some embodiments one or more operations of method 1300 is not performed.

At block 1302, processing logic receives scan data that includes at least one of a 3D model of a jaw of a patient, one or more 2D color intraoral images of the patient, or one or more NIRI intraoral images of the patient.

At block 1304, processing logic identifies a subset of the scan data for each tooth of the patient. Each subset of the can data can include a portion of the one or more 2D color intraoral images that corresponds to the tooth, a portion of the one or more NIRI intraoral images that corresponds to the tooth, and/or a secondary 3D model that corresponds to the tooth.

In some embodiments, to identify the subset of the scan data for each tooth of the patient, processing logic segments the 3D model to generate a series of secondary 3D models. Each secondary 3D model in the series of secondary 3D models corresponds to a tooth of the patient. In some embodiments, processing logic performs semantic segmentation of the 3D model of the jaw of the patient. For example, processing logic can use point cloud or mesh-based neural networks (e.g., sparse convolutional neural networks) to assign semantic labels to each point or vertex in the 3D dataset. The labels can distinguish between hard tissue (e.g., teeth) and soft tissues (e.g., gingiva, palate, tongue). The processing logic can partition the dental arch into discrete regions, each representing a single tooth, e.g., based on spatial continuity, morphological features, and/or anatomical landmarks. The processing logic can extract the subset of points or mesh elements associated with each labeled tooth region and reconstruct a secondary 3D model for each tooth that preserves its unique geometry and surface topology. In some embodiments, the processing logic can calibrate each secondary 3D model within a standardized coordinate system to maintain their spatial relationship to the jaw curve.

In some embodiments, processing logic can identify a portion of the one or more 2D color intraoral images corresponding to the tooth of the patient. In some embodiments, metadata of a 2D intraoral image can include spatial coordinates, camera positioning data, and/or indexing information that directly associates specific image regions with particular teeth. In some embodiments, processing logic can use timestamp data to correlate when specific teeth were being scanned with corresponding 2D image captures. In some embodiments, calibration parameters stored as metadata can provide the geometric relationship between the scanner position and the tooth being imaged. In some embodiments, processing logic can utilize sequence information that indicates which tooth was the focus of imaging during each 2D capture. In some embodiments, anatomical reference data embedded in the metadata can directly specify which portions of the 2D images correspond to which numbered teeth in the dental arch. Thus, in some embodiments, processing logic can identify a portion of the one or more 2D color intraoral images corresponding to the tooth of the patient using metadata.

In some embodiments, processing logic can utilize geometric calibration data to establish correspondence between the 3D model and the 2D color images. Processing logic can project the 3D segmentation results onto the 3D color images using intrinsic and extrinsic calibration parameters. The processing logic can apply transformation matrices to map 3D points to corresponding 2D pixel locations. The segmentation boundaries from the 3D model can be overlaid onto the 2D color images to delineate individual tooth regions. The processing logic may use direct geometric transformations based on the known spatial relationship between the 3D scanner and 2D camera sensors. This mapping process enables the system to identify which pixels in the 2D color images correspond to each segmented tooth from the 3D model.

In some embodiments, processing logic can perform direct 2D segmentation on the color intraoral image(s) using trained machine learning models, such as convolutional neural networks or U-Net architectures, to classify pixels and identify individual tooth regions. Processing logic can use image processing techniques such as edge detection, region growing, or watershed algorithms to delineate tooth boundaries. For instance, color-based analysis can help distinguish between tooth enamel, restorative materials, and surrounding soft tissues based on their optical properties. Processing logic can apply template matching algorithms that compare image regions against known tooth shape patterns stored in a database. In some embodiments, texture analysis can differentiate between natural tooth structure and artificial materials. Processing logic can employ feature detection algorithms that identify characteristic dental landmarks such as cusps, ridges, and contact points to segment individual teeth. Processing logic can employ clustering methods to group pixels with similar color and texture characteristics to define tooth regions. Processing logic can use morphological operations to refine segmentation boundaries and remove noise from the identified tooth portions.

In some embodiments, processing logic can identify a portion of the one or more NIRI intraoral images corresponding to the tooth of the patient. In some embodiments, processing logic can utilize the same (or similar) geometric calibration data and transformation matrices used for 2D color images to map NIRI image regions to specific teeth. In some embodiments, processing logic can project 3D segmentation results onto the NIRI images using intrinsic and extrinsic calibration parameters. Processing logic can apply direct transformation based on the known spatial relationship between the NIRI sensor and the 3D scanner. Alternatively or additionally, processing logic can perform direct segmentation on the NIRI images using machine learning models trained specifically for near-infrared dental imaging. Processing logic can leverage the distinct optical signatures of different dental materials in NIRI wavelengths to distinguish between natural tooth structure and restorative materials. Edge detection algorithms can identify tooth boundaries based on the translucency patterns characteristic of natural teeth in NIRI imaging, in embodiments. In some embodiments, processing logic can use the opaque center and translucent circumference patterns visible in NIRI images to delineate individual tooth regions. In some embodiments, template matching algorithms can compare NIRI image features against known tooth characteristics in near-infrared. wavelengths.

At block 1306, processing logic determine one or more chart annotations for a dental chart of the patient by performing blocks 1308-1312. At block 1308, processing logic can perform, for each subset of the scan data, a shape analysis to identify a status of the tooth of the patient, e.g., as described with respect to shape analysis engine 328 of FIG. 3.

In some embodiments, to perform the shape analysis, processing logic can provide the subset of the scan data as input to an ML model that outputs one or more indicators of the status of the tooth. The one or more indicators can include at least one of a tooth number indicator, a primary tooth indicator, an abutment indicator, a scan-body indicator, a partially hatched tooth indicator, a bracket indicator, an attachment indicator, a retainer indicator, and/or a braces indicator, for example.

In some embodiments, to perform the shape analysis, processing logic can provide the subset of the scan data as input to an ML model that is trained to provide, as output, at least one of a tooth identification, an outlier shape, and/or one or more indicators of dental accessories. Processing logic can receive, as output from the ML model, the tooth identification, the outlier shape, and/or the one or more indicators of dental accessories. In some embodiments, process logic can provide the tooth identification and/or the outlier shape to a classifier. The classifier can output one or more indicators of the status of the tooth. The one or more indicators can include a tooth number indicator, a primary tooth indicator, an abutment indicator, a scan-body indicator, a preparation tooth indicator, and/or a partially hatched tooth indicator, for example. In some embodiments, processing logic can provide the one or more indicators of dental accessories as input to a second ML model. The second ML model can output one or more additional indicators that include a bracket indicator, an attachment indicator, a retainer indicator, and/or a braces indicator.

At block 1310, processing logic can perform, for each subset of the scan data, a teeth arrangement analysis to identify a positioning of the tooth of the patient, e.g., as described with respect to teeth arrangement analysis engine 327 of FIG. 3.

In some embodiments, to perform the teeth arrangement analysis, processing logic can generate, based on the 3D model, a jaw line reference curve for the patient. Processing logic can identify, based on segmentation data of the secondary 3D model, a location of the tooth. Processing logic can make a comparison of the location of the tooth to the jaw line reference curve. Processing logic can determine, based on the comparison, the positioning of the tooth. The positioning can include a regular positioning, an irregular positioning, and/or an indication of a missing tooth.

At block 1312, processing logic can perform, for each subset of the can data, a material classification of the tooth of the patient, e.g., as described with respect to material analysis engine 326 of FIG. 3.

In some embodiments, to perform the material classification, processing logic can provide the subset of the scan data as input to an ML model that outputs a classification of at least one of a partial restoration, a veneer, an inlay, an only, an overlay, a filling, a full restoration crown, or a bridge. In some embodiments, processing logic can segment the classification of the at least one of the partial restoration, the veneer, the inlay, the only, the overlay, the filling, the full restoration crown, or the bridge from the corresponding tooth. The segmentation can provide at least one of a location or a size corresponding to the classification of the at least one of the partial restoration, the veneer, the inlay, the only, the overlay, the filling, the full restoration crown, or the bridge.

In some embodiments, processing logic can provide, to a user device (e.g., computing device 360, 305, and/or oral state capture system 210), the one or more chart annotations to include in the dental chart of the patient. In some embodiments, processing logic can provide the one or more chart annotations to a dental practice management system.

In some embodiments, processing logic can provide the one or more chart annotations to a diagnostic system (e.g., oral health diagnostics system 215 of FIG. 2). An outcome of the diagnostic system can include the one or more chart annotations. The chart annotations provided as outcomes of the diagnostic system can encompass a wide range of clinically relevant dental findings, each optionally encoded with descriptors. For example, the system may annotate individual teeth with identifiers such as tooth numbering, and specify the presence of full-coverage restorations (e.g., porcelain-fused-to-metal crown, zirconia crown), partial restorations (e.g., mesio-occlusal-distal composite filling, buccal veneer, inlay, or onlay), or dental prosthetics (e.g., pontic, implant-supported crown, abutment, or scan body). In some embodiments, surface-level annotations can indicate the location and extent of restorative materials, such as a distolingual amalgam filling or a buccal composite resin restoration, with surface mapping referenced to standard dental nomenclature (e.g., mesial, distal, buccal, lingual, occlusal). In some embodiments, additional chart annotations may include detection of dental accessories (e.g., orthodontic brackets, aligner attachments, bonded retainers), identification of primary versus permanent dentition, and the presence of dental conditions such as caries, calculus, or enamel hypoplasia. In some embodiments, the diagnostic system can also flag missing teeth, note extractions, and record irregularities in tooth position or alignment relative to the jaw reference curve. Each annotation can include metadata such as confidence scores, provenance (e.g., scan date, modality), and, where applicable, temporal change indicators, thereby providing a comprehensive and structured representation of the patient's oral health status for integration into the dynamic dental chart.

In some embodiments, processing logic can identify, for a tooth of the patient, a caries indication identified by a caries diagnostic system. Responsive to determining that the one or more chart annotations for the tooth of the patient satisfies a caries condition, processing logic can provide an updated caries identification to the caries diagnostic system.

In some embodiments, responsive to identifying, based on the one or more chart annotations, that the tooth is a restoration, processing logic can provide an indication of the restoration to a timelapse diagnostic system.

In some embodiments, responsive to identifying, based on the one or more chart annotations, that the patient is in active orthodontic treatment, processing logic can provide an indication of the active orthodontic treatment to a timelapse diagnostic system.

In some embodiments, responsive to identifying, based on the one or more chart annotations, that the tooth is an implant, processing logic can provide an indication of the implant to a tooth strength assessment system.

In some embodiments, processing logic can determine an orthodontic treatment plan for the patient. The orthodontic treatment plan can include a first location of a dental attachment for the tooth of the patient. Processing logic can identify, based on the one or more chart annotations, a second location of the dental attachment for the tooth of the patient. Responsive to determining that the first location an the second location differ, processing logic can provide, to a user device (e.g., computing device 360, 305, and/or oral state capture system 210), an indication of a displacement of the dental attachment.

In some embodiments, processing logic can compare a first secondary 3D model corresponding to a first tooth of the patient to a second secondary 3D model corresponding to a second tooth of the patient. The second tooth is symmetrically opposite the first tooth in the jaw of the patient. Based on the comparison, processing logic can determine an asymmetry metric that represents a dissimilarity between the first tooth and the second tooth. Processing logic can add the asymmetry metric to at least one of the subset of the scan data for the first tooth or the subset of the scan data for the second tooth. In some embodiments, processing logic can store the asymmetry metric as additional metadata within the subset of scan data. For example, the asymmetry metric can be appended as a numerical value, and/or data field associated with the tooth's segmentation information. In some embodiments, the asymmetry metric can be integrated into the existing data structure containing the tooth's geometric properties, material classifications, and/or spatial coordinates. In some embodiments, the processing logic can embed the asymmetry metric alongside other derived features such as shape descriptors, surface area measurements, and/or volumetric estimates. This integration can enable subsequent analysis modules to access the asymmetry information when making classification decisions or validation assessments. In some embodiments, the asymmetry metric ca become part of the comprehensive data representation for each tooth, allowing downstream algorithms to consider symmetry relationships during processing. The added asymmetry data can be referenced by cross-validation modules, material analysis engines, and/or chart annotation systems to improve accuracy and reliability of tooth identifications.

In some embodiments, responsive to determining that the asymmetry metric satisfies a condition, processing logic can perform an error recovery operation corresponding to the dissimilarity between the first tooth and the second tooth. In some embodiments, the asymmetry metric quantifies the geometric differences between corresponding teeth on opposite sides of the dental arch. The asymmetry metric can represent surface normal consistency, feature-embedding cosine similarity, and/or other geometric measurements between the mirrored tooth models. The processing logic can calculate the asymmetry metric using iterative closest point registration or Procrustes alignment techniques. The asymmetry metric can include volumetric differences, surface area variations, or curvature discrepancies between the symmetric tooth pair. Higher asymmetry values can indicate the presence of restorations, dental work, or pathological conditions on one tooth but not its counterpart. The processing logic can use predetermined threshold values to classify asymmetry levels as normal, moderate, or significant. Normal asymmetry may reflect natural biological variation between symmetric teeth. Significant asymmetry may suggest the presence of crowns, fillings, or other restorative materials that alter the tooth's original shape. The asymmetry metric can serve as a validation tool for confirming or rejecting initial tooth classifications and material identifications.

In some embodiments, processing logic can determine that the asymmetry metric satisfies the condition when the asymmetry metric exceeds a predetermined threshold value indicating abnormal dissimilarity between symmetric teeth. In some embodiments, the threshold can be set based on expected natural variation between contralateral teeth in healthy patients. Additionally or alternatively, the condition can be triggered when the asymmetry metric falls below a minimum threshold, suggesting potential segmentation errors or misidentification. In some embodiments, processing logic may evaluate multiple asymmetry parameters simultaneously to determine if the overall condition is met.

In some embodiments, the error recovery operation can include re-examining the segmentation boundaries of both teeth to correct potential segmentation errors. Processing logic can apply refined segmentation algorithms or alternative machine learning models to improve tooth identification accuracy. In some embodiments, processing logic can flag the affected teeth for manual review by a dental practitioner as part of the error recovery operation. In some embodiments, the error recovery operation can involve reclassifying one or both teeth based on additional analysis of shape, material, and/or arrangement data. In some embodiments, processing logic may adjust confidence scores associated with the initial tooth classifications. In some embodiments, processing logic can use alternative validation techniques, such as comparison with additional symmetric tooth pairs or analysis of historical patient data. In some embodiments, processing logic can generate updated chart annotations reflecting the corrected tooth identifications or material classifications.

In some embodiments, processing logic can validate a first set of one or more chart annotations corresponding to a first tooth of the patient by comparing the first set of one or more chart annotations to a second set of chart annotations corresponding to a second tooth of the patient. the second tooth is symmetrically opposite to the first tooth in the jaw of the patient. In some embodiments, the validation can leverage contralateral symmetry by mirroring the segmented secondary 3D tooth model of the first tooth across a mid-sagittal jaw reference (arch) curve and registering the mirrored model to the symmetrically opposite tooth, e.g., using iterative closest point or procrustes alignment. The processing logic can determine similarity metrics (e.g., surface normal consistency, feature-embedding cosine similarity) and compare these to cues from registered 2D color and/or NIRI images mapped via 3D-2D back-projection. High geometric similarity (e.g., a geometric similarity score that exceeds a threshold value), optionally combined with matching material signatures (e.g., natural enamel translucency) increase the confidence in tooth ID, surfaces, and/or condition labels, whereas pronounced geometric and/or material asymmetry (e.g., opaque NIRI response indicating a crown, only, or significant filling on buccal/occlusal surfaces) supports restoration annotations on only one side. In some embodiments, the processing logic can upweight or downweight confidence scores of the first tooths'chart annotations, auto-correct tooth ID or restoration type when contralateral evidence contradicts the initial classification, and/or flag likely false positives/negatives. The processing logic can perform cross-tooth consistency checks on a contralateral pair basis, accounting for expected asymmetric events (e.g., extractions, pontics, implants, preps, scan bodies).

In some embodiments, processing logic can validate a first set of the one or more chart annotations corresponding to a first scan of the patient taken at a first point in time by comparing the first set of the one or more chart annotations to a second set of chart annotations corresponding to a second scan of the patient taken at a second point in time. The second point in time predates the first point in time. In some embodiments, longitudinal analysis can leverage temporal consistency and change detection algorithms to identify clinically relevant differences, such as the appearance of new restorations, tooth extractions, and/or morphological changes due to dental procedures or disease progression. In some embodiments, the processing logic can utilize registration techniques (e.g., rigid or non-rigid alignment of 3D jaw models and corresponding teeth segmentation) to establish correspondence between teeth across timepoints. The processing logic can flag for review discrepancies in chart annotations, such as a transition from a natural tooth to a crown or the emergence of a missing tooth label. In some embodiments, the processing logic can annotate the discrepancies with provenance metadata indicating the time and nature of the detected change.

In some embodiments, processing logic can receive second scan data that includes one or more geometrical connections between the 3D model and 2D intraoral image(s) and/or the NIRI intraoral image(s). Responsive to segmenting the 3D model, processing logic can project segmentation results onto the 3D model and 2D intraoral image(s) and/or the NIRI intraoral image(s). Responsive to performing the shape analysis, processing logic can mpa the status of the tooth to the 3D model and 2D intraoral image(s) and/or the NIRI intraoral image(s). Responsive to performing the teeth arrangement analysis, processing logic can map the positioning of the tooth to the 3D model and 2D intraoral image(s) and/or the NIRI intraoral image(s). Responsive to performing the material classification, processing logic can map the composition of the tooth to the 3D model.

In some embodiments, to determine the one or more chart annotations for the dental chart of the patient, processing logic provides a secondary 3D model corresponding to the tooth of the patient, the one or more 2D color intraoral image(s), and/or the one or more NIRI intraoral image(s) as input to an ML model. The ML model can output one or more chart annotation indicators. The chart annotation indicator can correspond to a status indicator, a positioning indicator, and/or a composition indicator. In some embodiments, the status indicator can represent a tooth number, a primary tooth indicator, an abutment indicator, a scan-body indicator, a preparation tooth indicator, a partially hatched tooth indicator, a bracket indicator, an attachment indicator, a retainer indicator, and/or a braces indicator. In some embodiments, the positioning indicator can reflect a regular positioning, an irregular positioning, and/or an indication of a missing tooth. In some embodiments, the composition indicator can reflect a partial restoration, a veneer, an inlay, an only, an overlay, a filling, a full restoration crown, and/or a bridge.

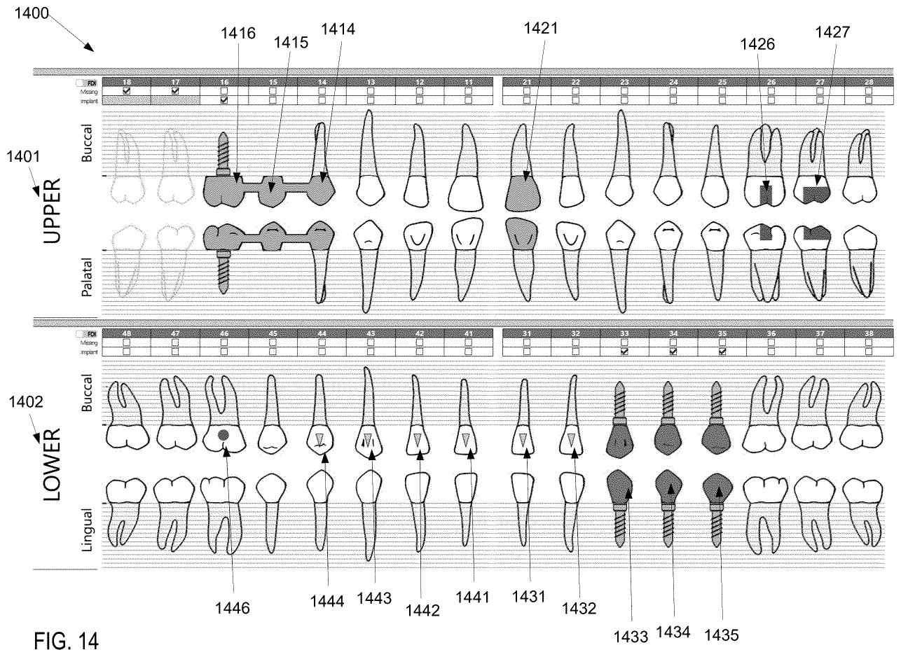

FIG. 14 illustrates an example of automatically generated chart annotations displayed in an example dental chart 1400, in accordance with embodiments of the present disclosure. In some embodiments, the dental chart 1400 can be presented in a UI of a user device (e.g., device 360, 305, and/or 210). FIG. 14 illustrates a dental chart 1400 that includes multiple chart annotations, 1414-1446. Dental chart 1400 include an upper jaw 1401 and lower jaw 1402. The upper jaw 1401 and the lower jaw 1402 each include buccal teeth and palatal (or lingual) teeth.

In some embodiments, chart annotations 1414-1446 can be automatically generated, e.g., by, auto charting system 316 of FIG. 3. In some embodiments, chart annotations 1414-1446 can be displayed in a different colors to indicate different findings. For example, annotations 1414-1421, indicating composite crowns, can be displayed in orange; annotations 1433-1435, indicating porcelain crowns, can be displayed in red; annotations 1426-27 and 1446, indicating fillings and/or partial restorations, can be displayed in blue; and annotations 1431-1444, indicating aligner attachments, can be displayed in green. In some embodiments, chart annotations 1414-1446 can be displayed using distinctive shapes to indicate the location of the findings. For example, aligner attachments corresponding to annotation 1431-1444 can be displayed using an arrow at or near the placement of the attachment on the tooth, and fillings and/or partial restorations corresponding to annotation 1426-1427 can be displayed using a rectangle at or near the location of the filling or restoration on the tooth. In some embodiments, crowns (e.g., porcelain crowns corresponding to annotations 1433-1435 and/or composite crowns corresponding to annotations 1414-1421) can be displayed by completely filling in the corresponding crown at the corresponding tooth location.

As illustrated in FIG. 14, chart annotation 1421 indicates that tooth 21 has a composite crown. Chart annotations 1414-1416 indicates that teeth 14-16 have a composite bridge. The composite bridge includes a partial bridge that covers a healthy tooth 14, a pontic (original tooth extract) tooth 15, and an implant at tooth 16. As illustrated in FIG. 14, chart annotations 1433-1435 indicate that the corresponding teeth (teeth 33-35) have porcelain crowns sitting on implants. As illustrated in FIG. 14, chart annotation 1446 indicates that tooth 46 has a buccal filling, chart annotation 1426 indicates that tooth 26 has an inlay, and chart annotation 1427 indicates that tooth 27 has an onlay. As illustrated in FIG. 14, chart annotations 1441-1444 and 1431-1432 indicate that teeth 41-44 and 31-32 have buccal attachments.

It should be noted that FIG. 14 illustrates only a subset of the possible chart annotations that can be generated by the auto charting system 316.

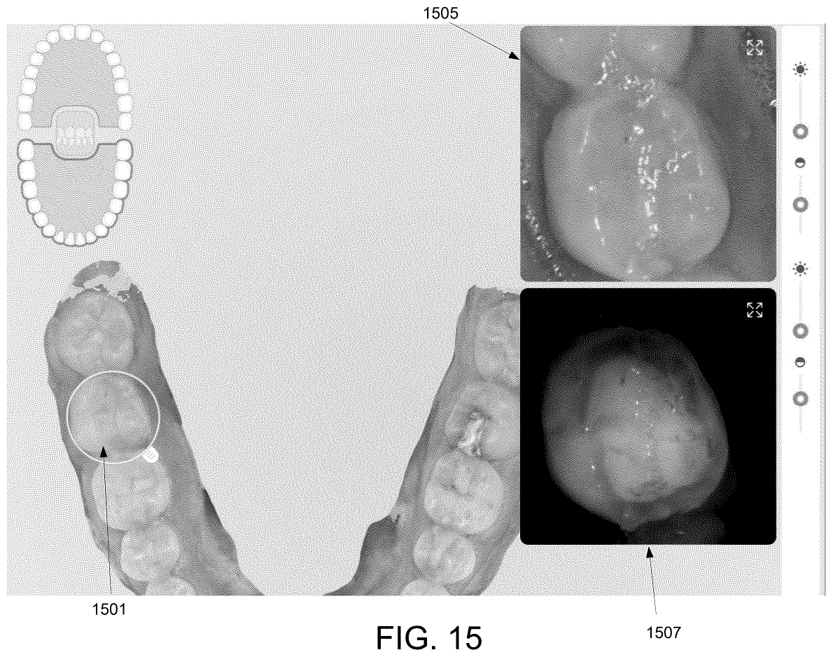

FIG. 15 illustrates example 2D color image 1505 and NIRI image 1507 depicting a healthy tooth 1501, in accordance with embodiments of the present disclosure. In some embodiments, the 2D color images 1505 and/or NIRI image 1507 can be provided to auto charting system 316. Auto charting system 316 can process the 2D color image 1505 and/or the NIRI image 1507, and can identify indicators of a healthy tooth, including, for example, the opaque center and translucent circumference in the NIRI image 1507. In some embodiments, auto charting system 316 can analyze image 1505 and/or 1507 to generate one or more chart annotations for tooth 1501. Auto charting system 316 may not generate any chart annotations for tooth 1501 since tooth 1501 is a healthy tooth (e.g., auto charting system 316 may not identify any indicators of anomalies warranting a chart annotation). In some embodiments, auto charting system 316 may generate an annotation to indicate the tooth 1501 as healthy.

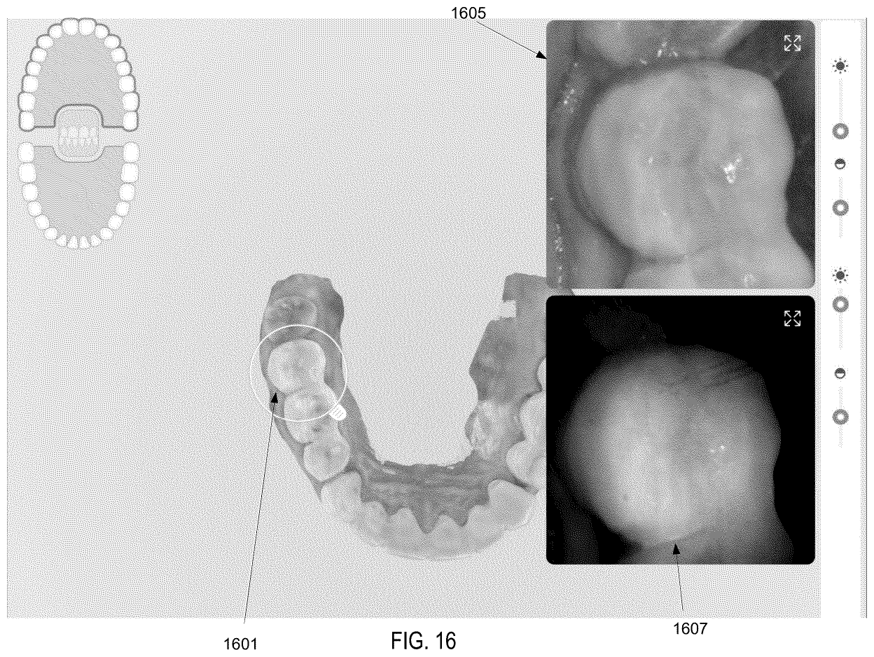

FIG. 16 illustrates example 2D color image 1605 and a NIRI image 1607 depicting a crown on tooth 1601, in accordance with embodiments of the present disclosure. The 2D color image 1605 can include an indication of crown, such as a color that is whiter than normal (e.g., the color differs from the color of the other teeth by a certain threshold amount). The NIRI image 1607 can include an indication of a crown, such as the tooth 1601 being opaque all over (e.g., does not have a translucent circumference). In some embodiments, auto charting system 316 can analyze image 1605 and/or 1607 to generate one or more chart annotations for tooth 1601. Auto charting system 316 may generate one or more chart annotations indicating tooth 1601 as having a crown. In some embodiments, the chart annotation(s) can include a tooth ID, a location of the crown (e.g., coordinates delineating the crown), a material of the crown, and so on.

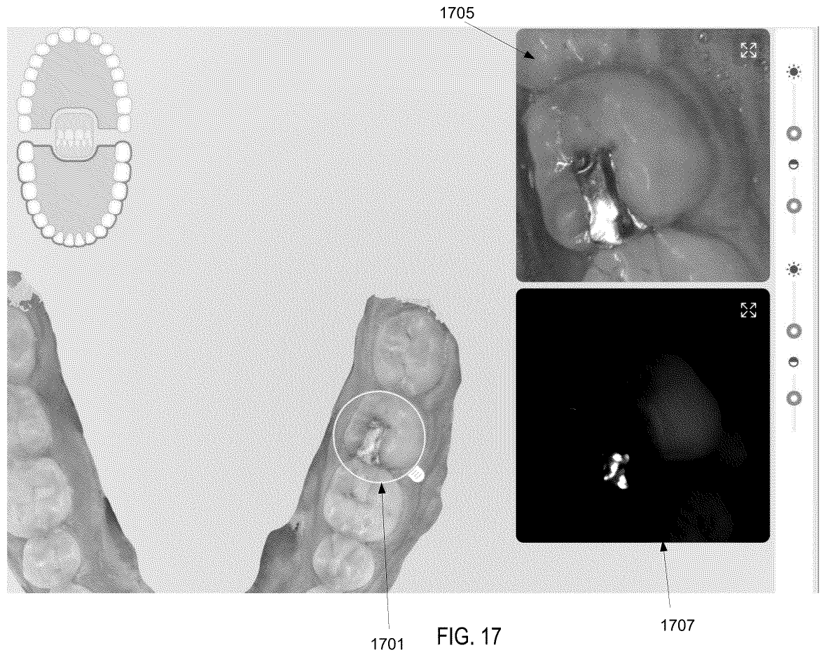

FIG. 17 illustrates example 2D color image 1705 and NIRI images 1707 depicting a metal filling on tooth 1701, in accordance with embodiments of the present disclosure. The 2D color image 1705 can include an indication of a metal filing, such as a distinct metal look (e.g., the color of the tooth satisfies a color criterion associated with metal fillings). The NIRI image 1707 can include an indication of a metal filling, such as the NIRI image being very dark (e.g., the brightness of the image satisfying a particular threshold). In some embodiments, the brightness and/or contrast of the NIRI image 1707 can be adjusted, and the metal filling may still appear as a hole on the tooth, and/or may appear shiny. In some embodiments, auto charting system 316 can analyze image 1705 and/or 1707 to generate one or more chart annotations for tooth 1701. Auto charting system 316 may generate one or more chart annotations indicating tooth 1701 as having a metal filling. In some embodiments, the chart annotation(s) can include a tooth ID, a location of the filling (e.g., coordinates delineating the filling), a material of the filling, and so on.

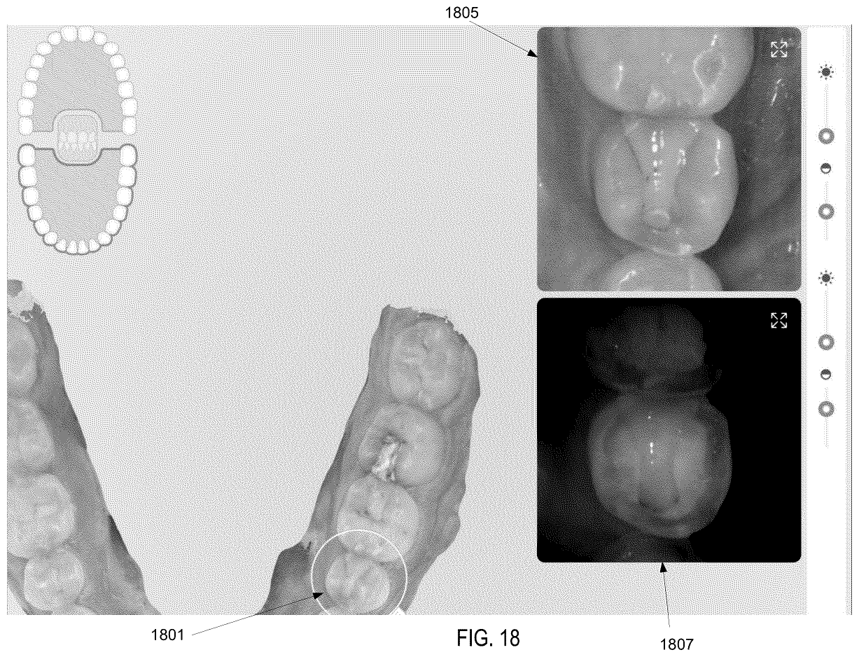

FIG. 18 illustrates example 2D color image 1805 and NIRI image 1807 depicting a composite filling on tooth 1801, in accordance with embodiments of the present disclosure. The 2D color image 1805 can include an indication of a composite filling, such as a an area that is whiter than natural teeth surrounding the filling. The NIRI image 1807 can include an indication the composite filling, such as a more opaque and more consistent look than the tooth center and/or having clear edges. In some embodiments, auto charting system 316 can analyze image 1805 and/or 1807 to generate one or more chart annotations for tooth 1801. Auto charting system 316 may generate one or more chart annotations indicating tooth 1801 as having a composite filling. In some embodiments, the chart annotation(s) can include a tooth ID, a location of the filling (e.g., coordinates delineating the filling), a material of the filing, and so on.

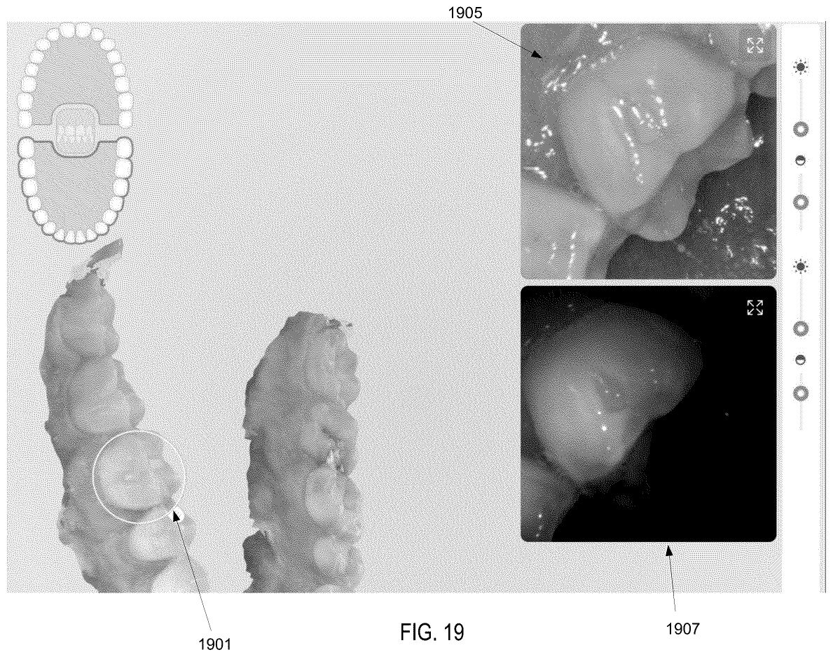

FIG. 19 illustrates example 2D color image 1905 and NIRI image 1907 depicting composite buccal filling on tooth 1901, in accordance with embodiments of the present disclosure. The 2D color image 1905 can include a minor indication of a buccal composite filling, such as irregularities at the edges of the filling. The NIRI image 1907 can include an indication of buccal composite filing, such as a clear variation of the filling area as compared to the rest of the tooth 1901. In some embodiments, auto charting system 316 can analyze image 1905 and/or 1907 to generate one or more chart annotations for tooth 1901. Auto charting system 316 may generate one or more chart annotations indicating tooth 1901 as having a buccal composite filling. In some embodiments, the chart annotation(s) can include a tooth ID, a location of the filling (e.g., coordinates delineating the filling), a material of the filling, and so on.

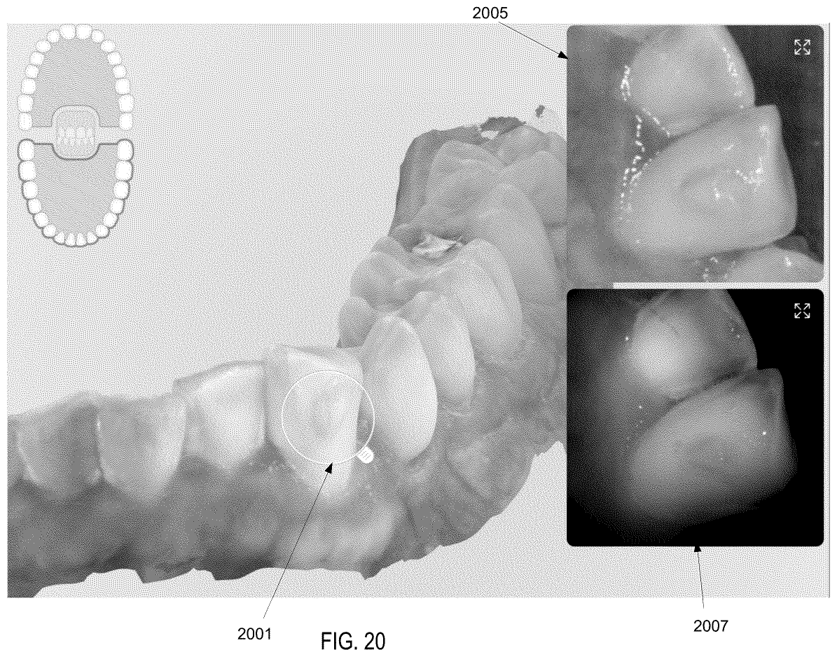

FIG. 20 illustrates example 2D color images 2005 and NIRI image 2007 depicting the presence of an aligner attachment on tooth 2001, in accordance with embodiments of the present disclosure. The 2D color image 2005 can include indications of the attachment, such as a whiter area than the natural tooth, and/or visible protrusion when viewed from the top view. The NIRI image 2007 can include an indication of the attachment, such as the area of the attachment being visually different from the rest of the tooth. In some embodiments, auto charting system 316 can analyze image 2005 and/or 2007 to generate one or more chart annotations for tooth 2001. Auto charting system 316 may generate one or more chart annotations indicating tooth 2001 as having an aligner attachment. In some embodiments, the chart annotation(s) can include a tooth ID, a location of the attachment (e.g., coordinates delineating the attachment), and so on.

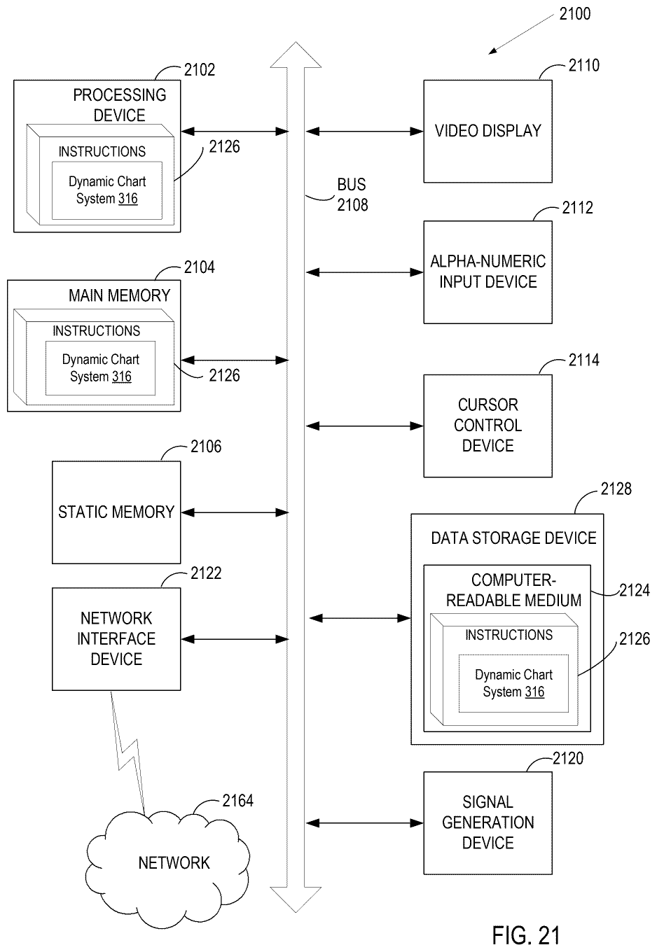

FIG. 21 illustrates a diagrammatic representation of a machine in the example form of a computing device 2100 within which a set of instructions, for causing the machine to perform any one or more of the methodologies discussed herein, may be executed. In alternative embodiments, the machine may be connected (e.g., networked) to other machines in a Local Area Network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein. In one embodiment, the computing device 2100 corresponds to any computing device of FIG. 3.

The example computing device 2100 includes a processing device 2102 (e.g., a CPU), a main memory 2104 (e.g., read-only memory (ROM), flash memory, dynamic random access memory (DRAM) such as synchronous DRAM (SDRAM), etc.), a static memory 2106 (e.g., flash memory, static random access memory (SRAM), etc.), and a secondary memory (e.g., a data storage device 2128), which communicate with each other via a bus 2108.

Processing device 2102 represents one or more general-purpose processors such as a microprocessor, central processing unit, or the like. More particularly, the processing device 2102 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 2102 may also be one or more special-purpose processing devices such as an application specific integrated circuit (ASIC), a field programmable gate array (FPGA), a digital signal processor (DSP), network processor, or the like. In accordance with one or more aspects of the present disclosure, processing device 2102 is configured to execute the processing logic (instructions 2126, which may implement the oral health diagnostic system 215 of FIG. 3) for performing operations and steps discussed herein. While only a single example processing device is illustrated, the term “processing device” shall also be taken to include any collection of processing devices (e.g., computers) that individually or jointly execute a set (or multiple sets) of instructions to perform any one or more of the methods discussed herein.

The computing device 2100 may further include a network interface device 2122 for communicating with a network 2164. The computing device 2100 also may include a video display unit 2110 (e.g., a liquid crystal display (LCD) or a cathode ray tube (CRT)), an alphanumeric input device 2112 (e.g., a keyboard), a cursor control device 2114 (e.g., a mouse, a touch-screen control device), and a signal generation device 2120 (e.g., a speaker).

The data storage device 2128 may include a machine-readable storage medium (or more specifically a non-transitory computer-readable storage medium) 2124 on which is stored one or more sets of instructions 2126 embodying any one or more of the methodologies or functions described herein. A non-transitory storage medium refers to a storage medium other than a carrier wave. The instructions 2126 may also reside, completely or at least partially, within the main memory 2104 and/or within the processing device 2102 during execution thereof by the computer device 2100, the main memory 2104 and the processing device 2102 also constituting computer-readable storage media.

The computer-readable storage medium 2124 may also be used to store an oral health diagnostic system 215, which may correspond to the similarly named component of FIG. 3. The computer readable storage medium 2124 may also store a software library containing methods for an oral health diagnostic system 215. While the computer-readable storage medium 2124 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any non-transitory medium (e.g., a medium other than a carrier wave) that is capable of storing or encoding a set of instructions for execution by the machine and that cause the machine to perform any one or more of the methodologies of the present disclosure. The term “computer-readable storage medium” shall accordingly be taken to include, but not be limited to, solid-state memories, and optical and magnetic media.

Any of the methods (including user interfaces) described herein may be implemented as software, hardware or firmware, and may be described as a non-transitory machine-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, computer models (e.g., for additive manufacturing) and instructions related to forming a dental device may be stored on a non-transitory machine-readable storage medium.

It should be understood that the above description is intended to be illustrative, and not restrictive. Many other embodiment examples will be apparent to those of skill in the art upon reading and understanding the above description. Although the present disclosure describes specific examples, it will be recognized that the systems and methods of the present disclosure are not limited to the examples described herein, but may be practiced with modifications within the scope of the appended claims. Accordingly, the specification and drawings are to be regarded in an illustrative sense rather than a restrictive sense. The scope of the present disclosure should, therefore, be determined with reference to the appended claims, along with the full scope of equivalents to which such claims are entitled.

The embodiments of methods, hardware, software, firmware, or code set forth above may be implemented via instructions or code stored on a machine-accessible, machine readable, computer accessible, or computer readable medium which are executable by a processing element. “Memory” includes any mechanism that provides (i.e., stores and/or transmits) information in a form readable by a machine, such as a computer or electronic system. For example, “memory” includes random-access memory (RAM), such as static RAM (SRAM) or dynamic RAM (DRAM); ROM; magnetic or optical storage medium; flash memory devices; electrical storage devices; optical storage devices; acoustical storage devices, and any type of tangible machine-readable medium suitable for storing or transmitting electronic instructions or information in a form readable by a machine (e.g., a computer).

Reference throughout this specification to “one embodiment” or “an embodiment” means that a particular feature, structure, or characteristic described in connection with the embodiment is included in at least one embodiment of the disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In the foregoing specification, a detailed description has been given with reference to specific exemplary embodiments. It will, however, be evident that various modifications and changes may be made thereto without departing from the broader spirit and scope of the disclosure as set forth in the appended claims. The specification and drawings are, accordingly, to be regarded in an illustrative sense rather than a restrictive sense. Furthermore, the foregoing use of embodiment, embodiment, and/or other exemplarily language does not necessarily refer to the same embodiment or the same example, but may refer to different and distinct embodiments, as well as potentially the same embodiment.

The words “example” or “exemplary” are used herein to mean serving as an example, instance, or illustration. Any aspect or design described herein as “example’ or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an embodiment” or “one embodiment” throughout is not intended to mean the same embodiment or embodiment unless described as such. Also, the terms “first,” “second,” “third,” “fourth,” etc. as used herein are meant as labels to distinguish among different elements and may not necessarily have an ordinal meaning according to their numerical designation.

A digital computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a digital computing environment. The essential elements of a digital computer a central processing unit for performing or executing instructions and one or more memory devices for storing instructions and digital data. The central processing unit and the memory can be supplemented by, or incorporated in, special purpose logic circuitry or quantum simulators. Generally, a digital computer will also include, or be operatively coupled to receive digital data from or transfer digital data to, or both, one or more mass storage devices for storing digital data, e.g., magnetic, magneto-optical disks, optical disks, or systems suitable for storing information. However, a digital computer need not have such devices.

Digital computer-readable media suitable for storing digital computer program instructions and digital data include all forms of non-volatile digital memory, media, and memory devices, including by way of example semiconductor memory devices, e.g., EPROM, EEPROM, and flash memory devices; magnetic disks, e.g., internal hard disks or removable disks; magneto-optical disks; CD-ROM and DVD-ROM disks.

Control of the various systems described in this specification, or portions of them, can be implemented in a digital computer program product that includes instructions that are stored on one or more non-transitory machine-readable storage media, and that are executable on one or more digital processing devices. The systems described in this specification, or portions of them, can each be implemented as an apparatus, method, or system that may include one or more digital processing devices and memory to store executable instructions to perform the operations described in this specification.

While this specification contains many specific embodiment details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. A system comprising:
a memory; and
a processing device to execute instructions from the memory to:
receive one or more datasets associated with a patient, wherein each dataset of the one or more datasets comprises data corresponding to an imaging modality of a plurality of imaging modalities and provides information on a dentition of the patient;
process the one or more datasets to generate, for each dataset of the one or more datasets, one or more tooth portions, wherein each tooth portion comprises an image of one or more teeth of the patient;
arrange the one or more tooth portions into a dental chart;
generate a visualization of the dental chart of the patient; and
provide, to a user device, the visualization of the dental chart for presentation in a user interface (UI).

28. The system of claim 27, wherein the processing device is further to:
normalize the one or more tooth portions for at least one of a size, a scale, a color balance, a brightness, or an orientation for coordinated presentation on the dental chart.

29. (canceled)

30. The system of claim 27, wherein the processing device is further to:
determine the imaging modality of the plurality of imaging modalities; and
select the one or more tooth portions corresponding to the determined imaging modality.

31. The system of claim 30, wherein to determine the imaging modality, the processing device is further to:
identify a correlation between a context of a dental visit and each of the plurality of imaging modalities;
rank each of the plurality of imaging modalities according to the correlation; and
determine the imaging modality with a highest ranking.

32. The system of claim 31, wherein the context of the dental visit corresponds to at least one of a complaint of the patient, an input of a user of the user device, a treatment plan, a treatment type, a scheduled dental visit, or a detection of a condition.

33. The system of claim 27, wherein the plurality of imaging modalities comprise at least one of: intraoral scan, near-infrared, cone beam computed tomography (CBCT), photograph, video, radiograph, fluorescence, or optical coherence tomography (OCT).

34. The system of claim 27, wherein the visualization of the dental chart comprises at least one of a buccal view, an occlusal view, or a lingual view of a dental arch of the patient.

35. The system of claim 27, wherein the visualization of the dental chart displays a dental arch of the patient in at least one of a two-dimensional view, a three-dimensional view, or a multi-dimensional view.

36. The system of claim 27, wherein the visualization comprises two or more imaging modalities.

37. The system of claim 27, wherein a first tooth portion of the one or more tooth portions has a first imaging modality and wherein a second tooth portion of the one or more tooth portions has a second imaging modality.

38. The system of claim 27, wherein the processing device is further to:
receive, from the user device, a user interaction associated with the visualization of the dental chart;
identify, based on the user interaction, a second imaging modality of the plurality of imaging modalities; and
update the visualization of the dental chart of the patient by selecting the one or more tooth portions that each corresponds to the second imaging modality.

39. The system of claim 27, wherein the processing device is further to:
process the at least one of the one or more datasets using a trained artificial intelligence (AI) model that outputs one or more detected oral conditions; and
update the one or more tooth portions to include a representation of the one or more detected oral conditions detected for one or more teeth represented in the one or more tooth portions.

40. The system of claim 27, wherein each dataset comprises data from a particular point in time.

41. The system of claim 27, wherein the visualization corresponds to a particular point in time.

42. The system of claim 27, wherein the UI enables a user of the user device to interact with the visualization by at least one of focusing in a particular section of a dental arch, changing imaging modalities, or changing a point in time.

43. The system of claim 27, wherein the processing device is further to:
provide, as input, the one or more datasets to an artificial intelligence model trained to provide an indication of a clinical finding corresponding to a particular imaging modality of the plurality of imaging modalities;
receive, from the artificial intelligence model, the indication of the clinical finding corresponding to the particular imaging modality; and
include the clinical finding in the visualization of the dental chart of the patient.

44. The system of claim 43, wherein to include the clinical finding in the visualization of the dental chart, the processing device is further to:
determine a second imaging modality of the plurality of imaging modalities other than the particular imaging modality; and
select the one or more tooth portions corresponding to the second imaging modality.

45. The system of claim wherein to determine the second imaging modality, the processing device is further to:
identify a correlation between the clinical finding and each of the plurality of imaging modalities;
rank each of the plurality of imaging modalities according to the correlation; and
determine the second imaging modality with a highest ranking.

46. The system of claim 27, wherein the processing device is further to:
receive, from at least one artificial intelligence model, a plurality of indications of a clinical finding, wherein each indication corresponds to a particular imaging modality of the plurality of imaging modalities.

47. The system of claim 46, wherein the processing device is further to:
identify a primary imaging modality corresponding to the clinical finding;
identify a primary indication of the plurality of indications, wherein the primary indication corresponds to the primary imaging modality; and
include the primary indication in the visualization of the dental chart of the patient.

48. The system of claim 46, wherein the processing device is further to:
generate an overall indication of the clinical finding by aggregating the plurality of indications of the clinical finding; and
include the overall indication of the clinical finding in the visualization of the dental chart of the patient.

49. (canceled)

50. The system of claim 27, wherein the data corresponding to the imaging modality comprises image data associated with a plurality of image sources, and wherein the processing device is further to:
provide, as input, the image data associated with the plurality of image sources to an artificial intelligence model trained to provide a confidence score associated with an identified clinical finding for each of the plurality of image sources;
receive, as output from the artificial intelligence model, a plurality of confidence scores associated with a clinical finding for the plurality of image sources;
identify an image source of the plurality of image sources with a highest confidence score; and
include, in the visualization of the dental chart, the image source with the highest confidence score.

51. The system of claim 27, wherein the visualization comprises a first portion displaying a dental arch of the patient in a panoramic format and a second portion displaying the dental arch of the patient in an arch format.

52. The system of claim 27, wherein the processing device is further to:
receive, from the user device, via one or more controls of the UI, a modification to a measurement of a tooth of the one or more teeth of the patient;
generate one or more updated tooth portions to reflect the modification;
generate an updated visualization of the dental chart to include the one or more updated tooth portions; and
provide, to the user device, the updated visualization of the dental chart.

53. A non-transitory computer-readable storage medium comprising instructions that, when executed by a processing device, cause the processing device to:
receive one or more datasets associated with a patient, wherein each dataset of the one or more datasets comprises data corresponding to an imaging modality of a plurality of imaging modalities and provides information on a dentition of the patient;
process the one or more datasets to generate, for each dataset of the one or more datasets, one or more tooth portions, wherein each tooth portion comprises an image of one or more teeth of the patient;
arrange the one or more tooth portions into a dental chart;
generate a visualization of the dental chart of the patient; and
provide, to a user device, the visualization of the dental chart for presentation in a user interface (UI).

54. (canceled)

55. (canceled)

56. (canceled)

57. The non-transitory computer-readable storage medium of claim 53, wherein to determine the imaging modality, the processing device is further to:
identify a correlation between a context of a dental visit and each of the plurality of imaging modalities, wherein the context of the dental visit corresponds to at least one of a complaint of the patient, an input of a user of the user device, a treatment plan, a treatment type, a scheduled dental visit, or a detection of a condition;
rank each of the plurality of imaging modalities according to the correlation;
determine the imaging modality of the plurality of imaging modalities with a highest ranking; and
select the one or more tooth portions corresponding to the determined imaging modality.

58. (canceled)

59. (canceled)

60. (canceled)

61. (canceled)

62. (canceled)

63. (canceled)

64. The non-transitory computer-readable storage medium of claim 53, wherein the processing device is further to:
receive, from the user device, a user interaction associated with the visualization of the dental chart;
identify, based on the user interaction, a second imaging modality of the plurality of imaging modalities; and
update the visualization of the dental chart of the patient by selecting the one or more tooth portions that each corresponds to the second imaging modality.

65. The non-transitory computer-readable storage medium of claim 53, wherein the processing device is further to:
process the at least one of the one or more datasets using a trained artificial intelligence (AI) model that outputs one or more detected oral conditions; and
update the one or more tooth portions to include a representation of the one or more detected oral conditions detected for one or more teeth represented in the one or more tooth portions.

66. (canceled)

67. (canceled)

68. (canceled)

69. The non-transitory computer-readable storage medium of claim 53, wherein the processing device is further to:
provide, as input, the one or more datasets to an artificial intelligence model trained to provide an indication of a clinical finding corresponding to a particular imaging modality of the plurality of imaging modalities;
receive, from the artificial intelligence model, the indication of the clinical finding corresponding to the particular imaging modality; and
include the clinical finding in the visualization of the dental chart of the patient.

70. (canceled)

71. (canceled)

72. The non-transitory computer-readable storage medium of claim 53, wherein the processing device is further to:
receive, from at least one artificial intelligence model, a plurality of indications of a clinical finding, wherein each indication corresponds to a particular imaging modality of the plurality of imaging modalities;
identify a primary imaging modality corresponding to the clinical finding;
identify a primary indication of the plurality of indications, wherein the primary indication corresponds to the primary imaging modality; and
include the primary indication in the visualization of the dental chart of the patient.

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. (canceled)

135. (canceled)

136. (canceled)

137. (canceled)

138. (canceled)

139. (canceled)

140. (canceled)

141. (canceled)

142. (canceled)

143. (canceled)

144. (canceled)

145. (canceled)

146. (canceled)

147. (canceled)

148. (canceled)

149. (canceled)

150. (canceled)

151. (canceled)

152. (canceled)

153. (canceled)

154. (canceled)

155. (canceled)

156. (canceled)

157. (canceled)

158. (canceled)

159. (canceled)

160. (canceled)

161. (canceled)

162. (canceled)

163. (canceled)

164. (canceled)

165. (canceled)
